(19) United States
(12) Patent Application Publication
(10) Pub. No.: US 2025/0320260 A1
(43) Pub. Date: Oct. 16, 2025

(54) PROTEINS TOXIC TO HEMIPTERAN INSECT SPECIES

(71) Applicant: Monsanto Technology LLC, St. Louis, MO (US)

(72) Inventors: James A. Baum, Webster Groves, MO (US); Artem G. Evdokimov, Orchard Park, NY (US); Stanislaw Flasinski, Ballwin, MO (US); Farhad Moshiri, Chesterfield, MO (US); Timothy J. Rydel, St. Charles, MO (US); Eric J. Sturman, Wildwood, MO (US); Moritz von Rechenberg, Waltham, MA (US); Halong Vu, Arlington, MA (US); Andrew M. Wollacott, Boston, MA (US); Meiying Zheng, Wildwood, MO (US)

(21) Appl. No.: 19/198,800

(22) Filed: May 5, 2025

Related U.S. Application Data
(62) Division of application No. 18/595,330, filed on Mar. 4, 2024, now Pat. No. 12,319,716.
Application No. 18/595,330 is a division of application No. 17/742,215, filed on May 11, 2022, now Pat. No. 11,981,709.
Application No. 17/742,215 is a continuation of application No. 17/127,310, filed on Dec. 18, 2020, now Pat. No. 11,459,359.
Application No. 17/127,310 is a continuation of application No. 16/594,713, filed on Oct. 7, 2019, now Pat. No. 10,897,910.
Application No. 16/594,713 is a continuation of application No. 16/209,501, filed on Dec. 4, 2018, now Pat. No. 10,485,238.
Application No. 16/209,501 is a continuation of application No. 15/651,727, filed on Jul. 17, 2017, now Pat. No. 10,188,115.
Application No. 15/651,727 is a division of application No. 15/015,957, filed on Feb. 4, 2016, now Pat. No. 9,713,334.
Application No. 15/015,957 is a continuation of application No. 13/857,196, filed on Apr. 5, 2013, now Pat. No. 9,322,033.
(60) Provisional application No. 61/621,436, filed on Apr. 6, 2012.

(51) Int. Cl. C07K 14/325; A01N 37/46; C07K 14/00; C07K 14/32; C07K 14/435; C12N 15/82

(52) U.S. Cl. CPC C07K 14/325; A01N 37/46; C07K 14/001; C07K 14/32; C07K 14/43577; C12N 15/8286; Y02A 40/146

(57) ABSTRACT

The present invention discloses Hemipteran insect inhibitory proteins, methods of using such proteins, nucleotide sequences encoding such proteins, methods of detecting and isolating such proteins, and their use in agricultural systems.

### CROSS REFERENCE TO RELATED APPLICATIONS

This application is a divisional of U.S. patent application Ser. No. 18/595,330, filed Mar. 4, 2024, which is a divisional of U.S. patent application Ser. No. 17/742,215, filed May 11, 2022, now U.S. Pat. No. 11,981,709, which is a continuation of U.S. patent application Ser. No. 17/127,310, filed Dec. 18, 2020, now U.S. Pat. No. 11,459,359, which is a continuation of U.S. patent application Ser. No. 16/594,713, filed Oct. 7, 2019, now U.S. Pat. No. 10,897,910, which is a continuation of U.S. patent application Ser. No. 16/209,501, filed Dec. 4, 2018, now U.S. Pat. No. 10,485,238, which is a continuation of U.S. patent application Ser. No. 15/651,727, filed Jul. 17, 2017, now U.S. Pat. No. 10,188,115, which is a divisional of U.S. patent application Ser. No. 15/015,957, filed Feb. 4, 2016, now U.S. Pat. No. 9,713,334, which is a continuation of U.S. patent application Ser. No. 13/857,196, filed Apr. 5, 2013, now U.S. Pat. No. 9,322,033, which claims the benefit of priority to U.S. Provisional Application Ser. No. 61/621,436 filed Apr. 6, 2012, each of the disclosures of which are incorporated herein by reference in their entirety.

### INCORPORATION OF SEQUENCE LISTING

The Sequence Listing contained in the file named “MONS514USD3_ST26.xml”, which is 395,049 bytes in size (measured in operating system MS-Windows®) and was created on May 2, 2025, is contemporaneously filed by electronic submission (using the United States Patent Office Patent Center filing system) and is incorporated herein by reference in its entirety.

### FIELD OF THE INVENTION

The present invention generally relates to the field of insect inhibitory proteins. In particular, the present invention relates to proteins exhibiting insect inhibitory activity against agriculturally relevant pests of crop plants and seeds, particularly Hemipteran species of insect pests.

### BACKGROUND OF THE INVENTION

Insect inhibitory proteins derived from Bacillus thuringiensis (Bt) are non-toxic to humans, vertebrates, and plants. These proteins are also biodegradable, safe, and effective in controlling pest insects. Some of these proteins have been and are being used to control agriculturally relevant pests of crop plants by spraying plants with formulations containing these proteins or with microorganisms that express them, treating seeds with treatments containing these proteins, or expressing these proteins in crop plants and seeds of crop plants as plant-incorporated protectants.

Certain Hemiptera species, particularly Amrasca, Empoasca and Lygus bugs, are pests of cotton and alfalfa, and typically are only controlled using broad spectrum chemistries, e.g., endosulfan, acephate, and oxamyl, which can persist in and are harmful to the environment. A few Bt proteins have been developed in formulations or as transgenic traits in crop plants for commercial use by farmers to control Coleopteran and Lepidopteran pest species, but no Bt proteins have been developed for use in commercial control of Hemipteran pest species.

Hemipteran specific toxic proteins have been reported in the art. TIC807 is a Bacillus thuringiensis protein disclosed in U.S. Patent Application Publication No. US 2008-0295207 A1 as being toxic to Hemipteran pest species. A Cry51Aa1 protein reported as toxic to Lepidopteran species that closely resembles the amino acid sequence of TIC807 has also been disclosed (Huang et al., (2007) J. Invertebr. Pathol. 95(3), 175-180), but no Hemipteran specific activity was reported. Baum et al. disclosed TIC853, a protein reported to be toxic to Lygus pest species (U.S. Patent Application Publication No. US 2010-0064394 A1). A protein referred to as AXMI-171 was reported to exhibit some limited inhibition of Hemipteran insects (U.S. Patent Application Publication No. US2010-0298207 A1, example 18), particularly Lygus hesperus.

All of these proteins exhibit a narrow range of toxicity only against Lygus hesperus and exhibit toxic effects against other Lygus pest species only in high doses which are not considered to be achievable by expression in plants. Compared to the Hemipteran toxic proteins in the prior art, there is a need for toxin proteins that can be used on and in plants that exhibit a broad host range against Hemipteran pest species and at low concentration effective doses.

### BRIEF SUMMARY OF THE INVENTION

Recombinantly engineered Hemipteran toxic proteins described herein (referred to herein as “engineered toxin proteins”, “engineered toxic proteins”, “engineered Hemipteran toxic proteins”, or “engineered Hemipteran toxin proteins”, are also referred to herein in truncated form as “eHTP's” when referred to in groups of two or more such proteins, and “eHTP” when referred to singularly) are derivatives of naturally occurring Bacillus thuringiensis insecticidal toxins, TIC807 (SEQ ID NO:2), TIC807 M2 (SEQ ID NO: 8), Cry51Aa1 (SEQ ID NO:182), TIC853 (SEQ ID NO:184), and AXMI-171 (SEQ ID NO:206) have been described previously to exhibit bio-control activity directed to Hemipteran pest species, particularly Lygus hesperus insect species (references cited elsewhere herein). The recombinant Hemipteran insect toxic proteins of the present invention are particularly toxic to insects of the Amrasca, Empoasca and Lygus species of insect pests and to other insect pest species that are phylogenetically related to each of these species of insect pests, and additionally to insect pests that feed on plants using a piercing and sucking mechanism used by the pest species Amrasca, Empoasca and Lygus species of the order Hemiptera. Unlike the precursor insecticidal toxins TIC807 (SEQ ID NO:2), TIC807 M2 (SEQ ID NO:8), Cry51Aa1 (SEQ ID NO: 182), TIC853 (SEQ ID NO:184), and AXMI-171 (SEQ ID NO:206) from which they are derived, which each require moderately high to high doses of protein to achieve toxic effects upon one Lygus species and exhibit very low or virtually undetectable toxic effects upon a second closely related species of Lygus, the eHTP proteins of the present invention exhibit surprising and unexpected low dose toxic effects against insect pests of the order Hemiptera, including host range toxic effects that span the spectrum of pests within the order.

The eHTP's of the present invention each contain at least one amino acid substitution, one amino acid addition, or one amino acid deletion compared to the primary amino acid sequence of one or more of the toxin proteins set forth in any of SEQ ID NO:2, SEQ ID NO:8, SEQ ID NO:182, or SEQ ID NO:184. In certain embodiments, an eHTP is provided that contains at least from about 2 to about 260 fold greater inhibitory activity against a Lygus pest species than any one or more of the toxins set forth in any of SEQ ID NO: 2 (TIC807), SEQ ID NO:8 (TIC807 M2), SEQ ID NO:182 (Cry51Aa1), SEQ ID NO:184 (TIC853), and/or SEQ ID NO:206 (AXMI-171). Optionally the eHTP exhibits at least about 95% amino acid sequence identity to the toxin protein selected from the group consisting of SEQ ID NO:2 (TIC807) and SEQ ID NO: 182 (Cry5 1Aa1). In certain embodiments, an eHTP is provided that contains at least one amino acid substitution, at least one amino acid addition, or at least one amino acid deletion when compared to the amino acid sequence of any of SEQ ID NO:2, SEQ ID NO:8, SEQ ID NO:182, or SEQ ID NO:184. The eHTP exhibits an increased or greater Lygus inhibitory activity and target pest species spectrum compared to the activity and target pest species spectrum of the Bacillus thuringiensis proteins of set forth in SEQ ID NO: 2, SEQ ID NO:8, SEQ ID NO: 182, and SEQ ID NO:184. Each of the aforementioned eHTP's contain at least, collectively or in the alternative: (i) the amino acid substitution, addition, or deletion in a solvent accessible amino acid residue of SEQ ID NO:2; (ii) the amino acid substitution, addition, or deletion within 3 consecutive residues of a solvent accessible amino acid residue of SEQ ID NO:2; or, (iii) an amino acid sequence as set forth in SEQ ID NO:180. The aforementioned eHTP's will each contain at least, with reference to the amino acid sequence positions as numbered according to the amino acid positions of TIC807, one substitution or deletion selected from the group consisting of asparagine at position 12 replaced by aspartic acid, phenylalanine at position 46 replaced by serine, isoleucine at position 52 replaced by methionine, tyrosine at position 54 replaced by histidine, threonine at position 68 replaced by alanine, glutamine at position 70 replaced by alanine, alanine at position 87 replaced by serine, threonine at position 93 replaced by alanine, serine at position 95 replaced by alanine, glycines at position 105 replaced by alanine, serine at position 117 replaced by alanine, serine at position 119 replaced by alanine, glutamate at position 125 replaced by cysteine, histidine, arginine, phenylalanine, serine, glutamine, lysine, threonine, asparagine, alanine, leucine, valine, methionine, aspartic acid, or tyrosine, glycines at position 128 replaced by alanine, threonine at position 133 replaced by glutamic acid, tyrosine, or tryptophan, isoleucine at position 134 replaced by alanine, valine, leucine, phenylalanine, lysine, cysteine, or methionine, glutamate at position 135 replaced by serine, alanine, valine, tryptophan, or threonine, asparagine at position 137 replaced by histidine, tyrosine, threonine, glutamic acid, serine, alanine, glutamine, glycine, isoleucine, tryptophan, lysine, cysteine, methionine, aspartic acid, phenylalanine, or arginine, phenylalanine at position 138 replaced by valine, Ala139 replaced by serine, Thr145 replaced by alanine, Phe 147 replaced by serine, valine, threonine, cysteine, leucine, aspartic acid, alanine, glycine, glutamic acid, isoleucine, tyrosine, methionine, asparagine, glutamine, hystidine, alanine, arginine, tryptophan, or proline, glutamine at position 148 replaced by alanine, glutamine at position 149 replaced by aspartic acid, glutamic acid, cysteine, alanine, or phenylalanine, alanine at position 150 replaced by serine, leucine, valine, glycine, aspartic acid, tryptophan, glutamic acid, asparagine, tyrosine, phenylalanine, proline, lysine, threonine, glutamine, or arginine, seroine at position 151 replaced by alanine, aspartate at position 153 replaced by alanine, glutamate at position 155 replaced by cysteine, isoleucine, lysine, aspartic acid, histidine, tyrosine, glutamine, lysine, asparagine, threonine, alanine, phenylalanine, arginine, methionine, proline, tryptophan, serine, or valine, asparagine at position 157 replaced by cysteine, aspartic acid, tryptophan, tyrosine, methionine, alanine, phenylalanine, valine, leucine, proline, glutamic acid, threonine, glycine, isoleucine, or arginine, isoleucine at position 158 replaced by alanine, serine at position 159 replaced by alanine or threonine, serine at position 167 replaced by arginine or alanine, valine at position 175 replaced by alanine, methionine at position 177 replaced by alanine, asparagine at position 180 replaced by aspartic acid, threonine at position 182 replaced by alanine, leucine at position 187 replaced by alanine, histidine at position 196 deleted, tyrosine at position 197 deleted, serine at position 198 deleted, histidine at position 199 deleted, tyrosine at position 200 replaced by alanine, tyrosine at position 200 deleted, Ser201 replaced by alanine, serine at position 201 deletion, tryptophan at position 208 replaced by alanine, serine at position 217 replaced by asparagine, proline at position 219 replaced by arginine, tryptophan at position 223 replaced by tyrosine, phenylalanine at position 235 replaced by alanine, asparagine at position 239 replaced by alanine, aspartate at position 241 replaced by alanine, threonine at position 243 replaced by alanine, valine at position 244 replaced by isoleucine, threonine at position 245 replaced by alanine, tyrosine at position 246 replaced by phenylalanine, threonine at position 247 replaced by alanine or lysine, serine at position 249 replaced by alanine or arginine, valine at position 250 replaced by alanine, valine at position 251 replaced by alanine, serine at position 252 replaced by alanine, arginine at position 273 replaced by tryptophan, threonine at position 274 replaced by alanine, isoleucine at position 275 replaced by alanine, arginine at position 282 replaced by alanine, histidine at position 287 replaced by alanine or phenylalanine, serine at position 293 replaced by alanine, asparagine at position 295 replaced by alanine, glutamate at position 299 replaced by alanine, methionine at position 300 replaced by alanine, threonine at position 303 replaced by alanine, proline at position 305 replaced by alanine, isoleucine at position 306 replaced by alanine, and threonine at position 308 replaced by alanine, or wherein the protein comprises any combination of the referenced substitutions and/or deletions. eHTP's contain at least one amino acid substitution, one amino acid addition, or one amino acid deletion at an amino acid residue of SEQ ID NO:2, or the corresponding amino acid position of SEQ ID NO:8, SEQ ID NO:182, or SEQ ID NO:184, selected from the group consisting of (i) an amino acid residue having a relative solvent-accessibility of from at least about 15% to at least about 36%; and (ii) an amino acid residue located within a distance of about 3 consecutive residues from an amino acid having from at least about 15% to at least about 36% relative solvent-accessibility. An eHTP of the present invention contains at least one amino acid substitution, addition, or deletion at an amino acid residue selected from the group consisting of Thr93, Ser95, Ser97, Phe147, Gln149, Ser151, Asn180, Thr182, VaI251, Gln253, and Ser255 of SEQ ID NO:2. Any of the aforementioned eHTP's can contain at least one additional amino acid substitution, addition, or deletion at an amino acid residue selected from the group consisting of Vail°, 11e14, Asn22, Asn23, Gly24, 11e25, Gln26, Gly27, Phe30, Gln38, 11e39, Asp40, Thr41, 11e43, Ser193, Thr194, Glu195, His196, Tyr197, Ser198, His199, Tyr200, Ser201, Gly202, Tyr203, Pro204, 11e205, Leu206, Thr207, Trp208, 11e209, Ser210, Tyr216, Ser217, Gly218, Pro219, Pro220, Met221, Ser222, Trp223, Tyr224, Phe225, Asn239, and Va1244 of SEQ ID NO: 2 or the corresponding amino acid residue position of SEQ ID NO:8, SEQ ID NO: 182, or SEQ ID NO:184. Any of the aforementioned eHTP's may contain one or more modifications selected from the group consisting of S95A, F147A, Q149E, V251A, P219R, and a deletion of any three consecutive amino acids from amino acid residues 196-201 as set forth in SEQ ID NO:2. Any of the eHTP's of the present invention can be further modified to exhibit increased solubility compared to the underlying naturally occurring Bacillus thuringiensis protein as set forth in any of SEQ ID NO:2, SEQ ID NO:8, SEQ ID NO: 182, or SEQ ID NO:184 in which the eHTP contains at least one or more amino acid sequence modifications relative to the amino acid sequence as set forth in SEQ ID NO:2. The modification(s) contain at least a lysine substitution at one or more of the amino acid positions defined as 58, 59, 198, 199, 201, or 202 in SEQ ID NO:2; a glutamic acid residue substitution at one or more of the amino acid positions defined as 198, 248, or 301 in SEQ ID NO:2; or an arginine residue substitution at one or more of the amino acid positions defined as 246, 250, or 253 in SEQ ID NO:2. An eHTP having an amino acid sequence selected from the group consisting of SEQ ID NO:6, SEQ ID NO:8, SEQ ID NO:10, SEQ ID NO:12, SEQ ID NO: 14, SEQ ID NO:16, SEQ ID NO:18, SEQ ID NO:20, SEQ ID NO:28, SEQ ID NO:30, SEQ ID NO:32, SEQ ID NO:34, SEQ ID NO:36, SEQ ID NO:37, SEQ ID NO:38, SEQ ID NO:39, SEQ ID NO:40, SEQ ID NO: 41, SEQ ID NO:42, SEQ ID NO:43, SEQ ID NO:44, SEQ ID NO:45, SEQ ID NO:46, SEQ ID NO: 47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO: 60, SEQ ID NO:61, SEQ ID NO:62, SEQ ID NO:63, SEQ ID NO:64, SEQ ID NO:65, SEQ ID NO: 66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO: 79, SEQ ID NO:80, SEQ ID NO:81, SEQ ID NO:82, SEQ ID NO:83, SEQ ID NO:84, SEQ ID NO: 85, SEQ ID NO: 86, SEQ ID NO: 87, SEQ ID NO:88, SEQ ID NO: 89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO: 98, SEQ ID NO:99, SEQ ID NO:100, SEQ ID NO:101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO:111, SEQ ID NO:112, SEQ ID NO: 113, SEQ ID NO:114, SEQ ID NO: 115, SEQ ID NO: 116, SEQ ID NO:117, SEQ ID NO:118, SEQ ID NO:119, SEQ ID NO: 120, SEQ ID NO: 121, SEQ ID NO: 122, SEQ ID NO:13, SEQ ID NO:124, SEQ ID NO:125, SEQ ID NO:126, SEQ ID NO:127, SEQ ID NO: 128, SEQ ID NO: 129, SEQ ID NO:130, SEQ ID NO: 131, SEQ ID NO: 132, SEQ ID NO:133, SEQ ID NO: 134, SEQ ID NO:135, SEQ ID NO:136, SEQ ID NO:137, SEQ ID NO:138, SEQ ID NO:139, SEQ ID NO: 140, SEQ ID NO:141, SEQ ID NO: 142, SEQ ID NO:143, SEQ ID NO:144, SEQ ID NO:145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO:154, SEQ ID NO: 155, SEQ ID NO:156, SEQ ID NO: 157, SEQ ID NO: 158, SEQ ID NO: 159, SEQ ID NO:160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO:168, SEQ ID NO:169, SEQ ID NO: 170, SEQ ID NO: 171, SEQ ID NO: 172, SEQ ID NO: 173, SEQ ID NO:174, SEQ ID NO:175, SEQ ID NO: 176, SEQ ID NO:177, SEQ ID NO:178, SEQ ID NO:179, SEQ ID NO:202, and SEQ ID NO:204, or an insect inhibitory fragment thereof, is a preferred embodiment of the present invention. The target Hemipteran pest species inhibited by the eHTP's of the present invention include at least Lygus hesperus, Lygus lineolaris, Empoasca fabae and Amrasca devastans, as well as other pests within the order Hemiptera that are phylogenetically related to each other or which use a piercing and sucking approach for feeding on plants.

Methods of controlling a Hemipteran pest by contacting the pest with a Hemipteran inhibitory amount of a eHTP of the present invention, as well as an insect inhibitory composition that contains at least a Hemipteran controlling amount (or Hemipteran inhibitory amount) of one or more of the eHTP's of the present invention, are also provided. In certain embodiments, an insect inhibitory composition comprising any of the eHTP's disclosed herein is provided. In certain embodiments of these methods, the Hemipteran pest is in a cotton field, a soybean field or an alfalfa field. Hemipteran toxic or Hemipteran controlling compositions can contain at least one or more eHTP along with a supplemental agent that is selected from the group consisting of an insect inhibitory protein, an insect inhibitory dsRNA molecule, and an insect inhibitory chemistry. Each of these agents can exhibit Hemipteran controlling properties, can exhibit properties for controlling pests unrelated to Hemipteran species such as Lepidopteran species or Coleopteran species, or may exhibit dual mode of action properties in which one or more Hemipteran species and one or more Lepidopteran or Coleopteran species are simultaneously controlled.

Recombinant polynucleotides that encode eHTP's of the present invention are provided. Microbes are also provided that contain the polynucleotides of the present invention, and such polynucleotides within such microbes are functionally positioned within expression cassettes designed to express the eHTP's of the present invention from operably linked functional genetic regulatory elements. Microbes are intended to include bacterial cells, as well as transgenic plant cells. Such transgenic plant cells can be regenerated into whole plants, or plant parts that also contain the recombinant polynucleotide. Methods of controlling a Hemipteran pest by exposing the pest to the microbe, whether bacterial cell or transgenic plant cell, plant or plant part, each of which expresses a Hemipteran inhibitory amount of an eHTP are also provided. The recombinant polynucleotide may contain a nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO: 27, SEQ ID NO:29, SEQ ID NO:31, SEQ ID NO:33, SEQ ID NO:35, SEQ ID NO:186, SEQ ID NO: 187, SEQ ID NO: 188, SEQ ID NO:189, SEQ ID NO: 190, SEQ ID NO:191, SEQ ID NO: 192, SEQ ID NO: 193, SEQ ID NO: 194, SEQ ID NO:195, SEQ ID NO: 196, SEQ ID NO:197, SEQ ID NO: 198, SEQ ID NO: 199, SEQ ID NO:200, SEQ ID NO:201, and SEQ ID NO:203, or other sequences that can be assembled to encode one or more of the proteins of the present invention. In certain embodiments, the recombinant polynucleotide can further comprise a nucleotide sequence encoding one or more insect inhibitory agents that are different from the eHTP encoded by the recombinant polynucleotide. The transgenic plant part is a seed, a boll, a leaf, a flower, pollen, a stem, a root, or any portion thereof. The transgenic plant part may be a non-regenerable portion of the seed, boll, leaf, flower, stem, or root. Also provided are methods of controlling a Hemipteran pest, comprising exposing the transgenic microbe, bacteria, plant cell, plant or plant part to the target pest, wherein the microbe, bacteria, plant cell, plant or plant part expresses a Hemipteran inhibitory amount of a eHTP encoded by the recombinant polynucleotide.

Processed plant products that contain a detectable amount of a recombinant polynucleotide encoding an eHTP or any distinguishing Hemipteran controlling portion thereof are also provided. Such processed products include, but are not limited to, plant biomass, oil, meal, animal feed, flour, flakes, bran, lint, hulls, and processed seed. The processed product may be non-regenerable.

Methods of making a transgenic plant by introducing the recombinant polynucleotide into a plant cell and selecting a transgenic plant that expresses an insect inhibitory amount of an eHTP encoded by a recombinant polynucleotide are also provided. The methods include introducing the recombinant polynucleotide encoding any of the eHTP's provided herein into a plant cell and selecting a transgenic plant that expresses an insect inhibitory amount of the eHTP encoded by the recombinant polynucleotide.

Other embodiments, features, and advantages of the invention will be apparent from the following detailed description, the examples, and the claims.

### BRIEF DESCRIPTION OF THE DRAWINGS

FIG. 1A illustrates the mortality of Lygus hesperus populations in response to various concentrations of four different eHTP's compared to a control sample containing the naturally occurring TIC807 protein. FIG. 1B illustrates the mortality of Lygus lineolaris populations in response to various protein concentrations of three different eHTP's compared to a control sample containing the naturally occurring TIC807 protein.

FIG. 2 illustrates a ribbon diagram of the atomic structure of a Hemipteran toxic protein of the present invention showing the relative positions of the result effective changes increasing toxic effects and/or broadening host range specificity compared to the relative position of the same amino acid position within a TIC807 or related protein. Two surface patches are illustrated by spheres encircling particular residue positions within the atomic structure in the ribbon diagram: [1] one sphere has an atomic radius of from about 9.2 to about 12.2 Angstroms from the beta carbon atom of S95 (relative to the S95 position as set forth in SEQ ID NO:2); [2] another sphere has an atomic radius of from about 9.2 to about 12.2 Angstroms from the beta carbon atom of P219 (relative to the P219 position as set forth in SEQ ID NO:2). Changes to the amino acids within the ribbon structure that fall within these spheres are result effective in causing increased toxic properties and broader host range toxic effects compared to a protein having a naturally occurring amino acid at that particular position.

FIG. 3 is a chart view illustrating the population mortality of Lygus species for thirteen different eHTP's compared to each other and to the naturally occurring TIC807 protein.

### DETAILED DESCRIPTION

This application describes eHTP's (engineered Hemipteran species toxic proteins). The eHTP's of the present invention are to be distinguished from proteins such as TIC807, TIC853, Cry51Aa1 and AXMI-171, which are known in the art and are not to be considered to be within the scope or definition of the term eHTP, as the prior art proteins are not engineered to exhibit improved toxic properties directed to one or more Hemipteran pest species and do not exhibit broad host range levels of inhibitory activity. eHTP's surprisingly and unexpectedly exhibit high levels of toxic activity against Hemipteran and related pest species. An additional feature of these eHTP's that is even more unexpected and surprising is the finding that these proteins exhibit broader host range toxic properties compared to progenitor proteins which provide the foundational basis for the eHTP's of the present invention. The foundational or baseline scaffold toxin proteins, such as TIC807 (SEQ ID NO:2), Cry51Aa1 (SEQ ID NO:8), TIC853 (SEQ ID NO:184), and AXMI-171 (SEQ ID NO:206) do not exhibit the breadth and scope of biological anti-Hemipteran activity or host range of the eHTP proteins of the present invention.

More than 2000 different amino acid sequence variants of Hemipteran toxic proteins derived from Bacillus thuringiensis species were tested to identify the specific amino acid insertions, substitutions, or deletions described herein which confer expanded Hemipteran species host range inhibitory spectrum and also provide dramatically increased Hemipteran species inhibitory activity when compared to the spectrum and activity of the baseline scaffold protein, TIC807, TIC853, and Cry51Aa1. Amino acid residues are identified in the baseline scaffold proteins that (a) can be modified to yield enhanced Hemipteran inhibitory spectrum and/or improved Lygus inhibitory activity relative to one or more of the scaffold proteins, (b) accumulate in surface patches of a folded insect inhibitory protein exhibiting the fold structure of one or more of the scaffold proteins, and/or (c) occur in specific positions of one or more of the scaffold protein amino acid sequence that are result effective in decreasing the resulting eHTP proteins' mean effective dose for controlling a Hemipteran species and broadening the range of Hemipteran species that are affected by the eHTP protein.

The Hemipteran pest species are intended to mean insects that feed upon plants and plant tissues by slashing or piercing the outer surface of the target plant, and then consume macerated plant exudates pooling in the slash or pierce location by sucking or wicking the pooled exudates. Such insects include adults and nymphs, including but not limited to the following listing of plant bugs: the Family Miridae, cicadas from the Family Cicadidae, leafhoppers (e. g., Empoasca spp., Amrasca spp.) from the Family Cicadellidae, planthoppers from the families Fulgoroidea and Delphacidae, treehoppers from the Family Membracidae, psyllids from the Family Psyllidae, whiteflies from the Family Aleyrodidae, aphids from the Family Aphididae, phylloxera from the Family Phylloxeridae, mealybugs from the Family Pseudococcidae, scales from the families Coccidae, Diaspididae and Margarodidae, lace bugs from the Family Tingidae, stink bugs from the Family Pentatomidae, cinch bugs (e. g., Blissus spp.) and other seed bugs from the Family Lygaeidae, spittlebugs from the Family Cercopidae squash bugs from the Family Coreidae, and red bugs and cotton stainers from the Family Pyrrhocoridae. Other pests from the order Hemiptera include Acrosternum hilare (green stink bug), Anasa tristis (squash bug), Blissus leucopterus leucopterus (chinch bug), Corythuca gossypii (cotton lace bug), Cyrtopeltis modesta (tomato bug), Dysdercus suturellus (cotton stainer), Euschistus servus (brown stink bug), Euschistus variolarius (one-spotted stink bug), Graptostethus spp. (complex of seed bugs), Leptoglossus corculus (leaf-footed pine seed bug), Lygus lineolaris (tarnished plant bug), Lygus hesperus (Western tarnish plant bug), Nezara viridula (southern green stink bug), Oebalus pugnax (rice stink bug), Oncopeltus fasciatus (large milkweed bug), and Pseudatomoscelis seriatus (cotton fleahopper). More specifically, the Family Cicadellidae includes, but is not limited to the tribe Empoascini, e.g. Amrasca biguttula, Amrasca devastans, Austroasca viridigrisea, Asymmetrasca decedens, Empoasca decipiens, Empoasca distinguenda, Empoasca dolichi, Empoasca fabae, Empoasca kerri, Empoasca kraemeri, Empoasca onukii, Empoasca sakaii, Empoasca smithi, Empoasca vitis, Jacobiasca lybica, Sonasasca Solana, tribe Erythroneurini, e.g. Empoascanara nagpurensis, Thaiaassamensis, Zygnidia quyumi, tribe Nirvaniae, e.g. Sophonia rufofascia, Family Delphacidae, e.g. Nilapoanvata lugens, Sogatella furcifera, Unkanodes sapporonus, and Family Lophopidae, e.g. Zophiuma lobulata.

eHTP's of the present invention contain one or more amino acid sequence modifications compared to one or more of the scaffold proteins, including substitutions and deletions, of amino acid residues at seventy-two (72) different amino acid positions. Such modifications provide eHTP's with increased toxicity and/or an enhanced inhibitory spectrum against Hemipteran insects when compared to one or more of the scaffold proteins which include but are not limited to TIC807 (SEQ ID NO:2), or related protein such as TIC807 M2 (SEQ ID NO:8), Cry51Aa1 (SEQ ID NO:182), and TIC853 (SEQ ID NO:184). eHTP's include, but are not limited to, modifications of at least one amino acid substitution or one amino acid deletion at any of these seventy-two positions, described as “X” in the amino acid sequence set forth as SEQ ID NO:180 but do not include the amino acid sequences of SEQ ID NO:2, SEQ ID NO:8, SEQ ID NO:182, or SEQ ID NO: 184. eHTP's of the present invention also exhibit enhanced Hemipteran inhibitory spectrum and/or improved Hemipteran inhibitory activity when compared to the spectrum and activity of the baseline or scaffold proteins.

eHTP's include at least one amino acid modification of the relative positions of TIC807 (SEQ ID NO: 2) as set forth above in paragraph [0009]. eHTP's can also include at least two, three, four, or more of these aforementioned amino acid substitutions and/or deletions and can also include at least two, three, four, or more of these amino acid substitutions and/or deletions as well as a deletion of any three contiguous amino acids within residues 196-201 of SEQ ID NO:2. Accordingly, eHTP's include proteins set forth as SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:28, SEQ ID NO:30, SEQ ID NO:32, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO: 43, SEQ ID NO:44, SEQ ID NO:45, SEQ ID NO:46, SEQ ID NO:47, SEQ ID NO:48, SEQ ID NO: 49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO: 62, SEQ ID NO:63, SEQ ID NO:64, SEQ ID NO:65, SEQ ID NO:66, SEQ ID NO:67, SEQ ID NO: 68, SEQ ID NO: 69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO: 81, SEQ ID NO:82, SEQ ID NO:83, SEQ ID NO:84, SEQ ID NO:85, SEQ ID NO:86, SEQ ID NO: 87, SEQ ID NO:88, SEQ ID NO: 89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO: 100, SEQ ID NO: 101, SEQ ID NO: 102, SEQ ID NO:103, SEQ ID NO: 104, SEQ ID NO:105, SEQ ID NO: 106, SEQ ID NO:107, SEQ ID NO: 108, SEQ ID NO: 109, SEQ ID NO:110, SEQ ID NO:111, SEQ ID NO: 112, SEQ ID NO:113, SEQ ID NO:114, SEQ ID NO:115, SEQ ID NO:116, SEQ ID NO: 117, SEQ ID NO: 118, SEQ ID NO:119, SEQ ID NO:120, SEQ ID NO:121, SEQ ID NO:122, SEQ ID NO:123, SEQ ID NO: 124, SEQ ID NO: 125, SEQ ID NO: 126, SEQ ID NO: 127, SEQ ID NO: 128, SEQ ID NO:129, SEQ ID NO: 130, SEQ ID NO:131, SEQ ID NO: 132, SEQ ID NO: 133, SEQ ID NO:134, SEQ ID NO: 135, SEQ ID NO: 136, SEQ ID NO: 137, SEQ ID NO: 138, SEQ ID NO: 139, SEQ ID NO:140, SEQ ID NO: 141, SEQ ID NO: 142, SEQ ID NO: 143, SEQ ID NO: 144, SEQ ID NO: 145, SEQ ID NO:146, SEQ ID NO:147, SEQ ID NO: 148, SEQ ID NO: 149, SEQ ID NO: 150, SEQ ID NO: 151, SEQ ID NO:152, SEQ ID NO:153, SEQ ID NO: 154, SEQ ID NO: 155, SEQ ID NO: 156, SEQ ID NO: 157, SEQ ID NO:158, SEQ ID NO:159, SEQ ID NO: 160, SEQ ID NO: 161, SEQ ID NO: 162, SEQ ID NO: 163, SEQ ID NO:164, SEQ ID NO:165, SEQ ID NO: 166, SEQ ID NO: 167, SEQ ID NO: 168, SEQ ID NO: 169, SEQ ID NO:170, SEQ ID NO:171, SEQ ID NO: 172, SEQ ID NO: 173, SEQ ID NO: 174, SEQ ID NO:175, SEQ ID NO:176, SEQ ID NO:177, SEQ ID NO: 178, SEQ ID NO:179, SEQ ID NO:202, and SEQ ID NO:204, and insect inhibitory fragments thereof.

eHTP's of the present invention exhibit any amino acid sequence different from any one or more of the scaffold proteins, including SEQ ID NO:2 (TIC807), in at least one amino acid position where the different amino acid residue either (i) has a relative amino acid solvent-accessibility of at least from about 15% to at least about 36% compared to the same residue positions in any one or more of the scaffold proteins; and/or (ii) is located within a distance of about 3 consecutive amino acid residues from an amino acid having at least from about 15% to at least about 36% relative solvent-accessibility compared to the corresponding amino acid residue positions in the primary amino acid sequence of one or more of the scaffold proteins, and exhibits broadened Hemipteran inhibitory spectrum and/or increased Hemipteran inhibitory activity when compared to the activity correlated with one or more of the scaffold proteins. The words “increased spectrum” are intended to mean, with reference to two different proteins exhibiting toxic effects to a particular single pest, the protein exhibiting increased spectrum exhibits toxic effects to that particular single pest as well as to one or more other pests within the same phylogenetic order or to one or more other pests in one or more different phylogenetic orders other than the order to which the particular single pest belongs. The words “increased Hemipteran inhibitory activity” are intended to mean that a particular protein exhibiting such increased activity requires, under standardized conditions, a lower amount of that protein to achieve a particular affect, such as mortality, stunting, morbidity, cessation of feeding, or another measureable phenotypic effect upon a particular single pest, than a control protein.

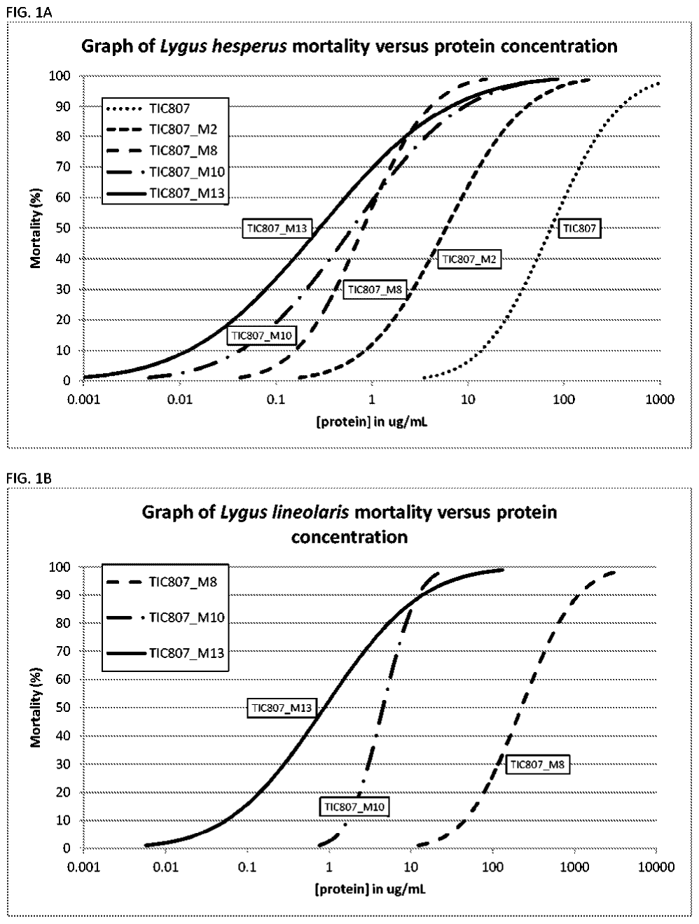

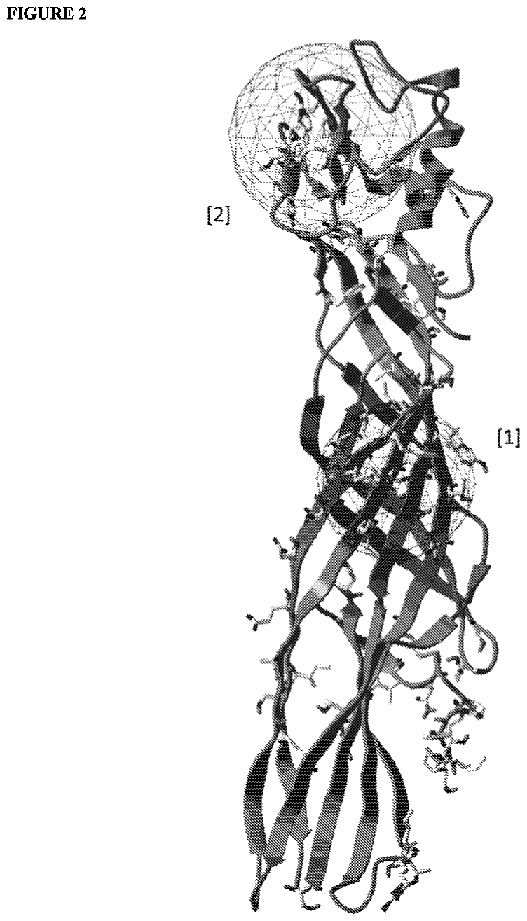

eHTP's exhibit an amino acid sequence that differs from one or more of the scaffold proteins, including particularly TIC807, in at least one amino acid residue located within at least one of the two different surface patches of a folded insect inhibitory protein (see FIG. 2 and Table 3 data). One surface patch is defined as including the amino acid residues encompassed within a sphere having an atomic radius of from about 9.2 to about 12.2 Angstroms (FIG. 2, sphere [1]) relative to the beta-carbon (Cb) atom of Ser95 as set forth in SEQ ID NO:2 when that protein is folded into a three dimensional structure under physiological conditions; which includes residues Thr93, Ser95, Ser97, Phe147, Gln149, Ser151, Asn180, Thr182, Va1251, Gln253, and Ser255. As used herein, the phrase “Cb atom” refers to the beta-carbon atom in the amino acid residue side chain. The Cb atom is thus the first carbon in the protein side chain that is present in all amino acid residues with the exception of Glycyl residues. With reference to FIG. 1, eHTP's can include, but are not limited to, one or more conservative or non-conservative substitutions of surface patch [1] amino acid residues T93, S95, S97, F147, Q149, S151, N180, T182, V251, Q253, and 5255 or the equivalent amino acids within one or more of the scaffold proteins, particularly SEQ ID NO:2 (TIC807). eHTP's can include, but are not limited to, one or more substitutions of surface patch [1] amino acid residues such as: T93A; S95A, S95V, S95L, or S951; F147T, F147C, F147D, F147G, F147E, F147Y, F147M, F147N, F147Q, F147H, F147R, F147W, F147P, F147A, F147V, F147L, or F1471; Q149A, Q149C, Q149F, Q149E or Q149D; S151A; N180D; T182A; V251E or V251A, and/or Q253R. The other or second surface patch that has been identified as amino acid residues that are receptive to modifications which are result effective in conferring improved Hemipteran inhibitory bioactivity in the form of eHTP's of the present invention is defined as including the amino acid residues encompassed within a sphere having an atomic radius of from about 9.2 to about 12.2 Angstroms (FIG. 2, sphere [2]) relative to the beta-carbon atom of Pro219 or the equivalent amino acid position in one or more of the scaffold proteins, particularly as set forth in SEQ ID NO:2, when any one of the applicable scaffold proteins is folded into a three dimensional structure under physiological conditions, which includes residues Vail°, Ile14, Asn22, Asn23, Gly24, 11e25, Gln26, Gly27, Phe30, Gln38, 11e39, Asp40, Thr41, 11e43, Ser193, Thr194, Glu195, His196, Tyr197, Ser198, His199, Tyr200, Ser201, Gly202, Tyr203, Pro204, 11e205, Leu206, Thr207, Trp208, 11e209, Ser210, Tyr216, Ser217, Gly218, Pro219, Pro220, Met221, Ser222, Trp223, Tyr224, Phe225, Asn239, and Va1244. Such eHTP's can include, but are not limited to, one or more conservative or non-conservative amino acid residues substitutions and/or one or more amino acid deletions within surface patch [2] including Vail°, Ile14, Asn22, Asn23, Gly24, Ile25, Gln26, Gly27, Phe30, Gln38, Ile39, Asp40, Thr41, Ile43, Ser193, Thr194, Glu195, His196, Tyr197, Ser198, His199, Tyr200, Ser201, Gly202, Tyr203, Pro204, Ile205, Leu206, Thr207, Trp208, Ile209, Ser210, Tyr216, Ser217, Gly218, Pro219, Pro220, Met221, Ser222, Trp223, Tyr224, Phe225, Asn239, and Va1244 of SEQ ID NO:2 (TIC807). eHTP's can include, but are not limited to, one or more substitutions and/or deletions within the amino acid residues located within surface patch [2] such as: a deletion of any three contiguous amino acid residues in the sequence His196, Tyr197, Ser198, His199, Tyr200, Ser201; Ser217Asn, Ser217Gln, Ser217Arg; and/or Pro219Arg, Pro219Asn, Pro219G1n. eHTP's can include, but are not limited to, one or more amino acid residue substitutions and/or deletions within surface patch [2] such as: a deletion of any three contiguous HisTyrSer residues in the sequence His196, Tyr197, Ser198, His199, Tyr200, Ser201; Ser217Asn, Ser217G1n, Ser217Arg; and/or Pro219Arg, Pro219Asn, Pro219G1n. An eHTP can have at least one amino acid modification in each of the two aforementioned surface patches of the folded insect inhibitory protein. eHTP can have one, or a combination of more than one modification at residues T93, S95, F147, Q149, 5151, N180, T182, H196, Y197, 5198, H199, Y200, 5201, W208, 5217, P219, W223, N239, V244, or V251 relative to SEQ ID NO:2 (TIC807). Conservative amino acid changes can be made by substituting an acidic, basic, neutral polar, or neutral non-polar-type amino acid with another amino acid of the same type. Non-conservative amino acid changes can be made by substituting an acidic, basic, neutral polar, or neutral non-polar amino acid-type with an amino acid of a different type. Furthermore, of the eHTP proteins listed in Table 4B, all 267 are amino acid sequence variants that exhibit increased toxicity to Lygus spp. when compared to one or more of the scaffold proteins, including scaffold protein TIC807. Only ten of these amino acid sequence variants exhibit modified amino acid residues compared to one or more of the scaffold proteins that are positioned outside of the two referenced surface patches.

The prior art teaches solubility problems associated with the scaffold proteins. eHTP's exhibit improved solubility compared to the scaffold proteins, and generally exhibit increased solubility at a pH of less than 9.0, in contrast to the observed solubility profile of one or more of the scaffold proteins. This increased solubility at more physiological pH is evident when the eHTP is expressed in E. coli, in a plant cell, in a plant cell cytoplasm, a plant cell apoplast, or in or targeted for import into a plastid of a plant cell. Amino acid modifications that improve solubility relative to one or more of the scaffold proteins, including SEQ ID NO:2 (TIC807) include but are not limited to, substitution of a lysine amino acid residue at one or more of the following amino acid positions in TIC807 or the applicable residue in any of the other scaffold proteins: 58, 59, 198, 199, 201, or 202; or, substitution of a glutamic acid amino acid residue at one or more of amino acid positions 198, 248 or 301; or, substitution of a arginine amino acid residue at one or more of amino acid positions 246, 250 or 253.

Insect inhibitory compositions comprising the above described eHTP's are also provided. Such compositions may further comprise at least one additional insect inhibitory agent different from the eHTP included in the composition. The insect inhibitory agent is selected from any number of insect inhibitory agents including an insect inhibitory protein, an insect inhibitory dsRNA molecule, and one or more chemical agents useful in controlling insect pests. Examples of additional inhibitory agents includes, but are not limited to, a TIC1415 protein, a dsRNA directed towards Hemipteran orthologs of Nilaparvata lugens V-ATPase-E, 21E01, a dsRNA directed towards Hemipteran orthologs of actin ortholog, ADP/ATP translocase, a-tubulin, ribosomal protein L9 (R PL9) or V-ATPase A subunit, AXMI-171 (US20100298207A1), Cry3A, Cry4Aa, CryllAa, and CytlAa, DIG11, DIGS, Cry7, eCry3.1Ab, mCry3A, Cry8, Cry34/Cry35, Cry3, DIG2, Cryl, Cry1A.105, Cry2, Cry1F, VIP3, 5307, and Cry9. Chemical agents useful in controlling Hemipteran species include but are not limited to pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates. Known pesticides within these categories are listed in The Pesticide Manual, llth Ed., C. D. S. Tomlin, Ed., British Crop Protection Council, Farnham, Surry, UK (1997).

Pyrethroids that are useful in the present composition include pyrethrins and synthetic pyrethroids. The pyrethrins that are preferred for use in the present method include, without limitation, 2-ally1-4-hydroxy-3-methyl-2-cyclopenten-l-one ester of 2,2-dimethy1-3-(2methyl propenyl)-cyclopropane carboxylic acid, and/or (2-methyl-l-propeny1)-2-methoxy-4-oxo-3-(2 propeny1)-2-cyclopenten-l-y1 ester and mixtures of cis and trans isomers thereof (Chemical Abstracts Service Registry Number (“CAS RN”) 8003-34-7).

Synthetic pyrethroids that are preferred for use in the present invention include (s)-cyano(3-phenoxyphenyl)methyl 4-chloro alpha (1-methylethyl)benzeneacetate (fenvalerate, CAS RN 51630-58-1), (S)-cyano (3-phenoxyphenyl) methyl (S)-4-chloro-alpha-(1-methylethyl) benzeneacetate (esfenvalerate, CAS RN 66230-04-4), (3-phenoxypheny1)-methyl(+)cis-trans-3-(2,2-dichoroetheny1)-2,2-dimethylcyclopropanecarboxylate (permethrin, CAS RN 52645-53-1), (±) alpha-cyano-(3-phenoxyphenyl) methyl(+)-cis,trans-3-(2,2-dichloroetheny1)-2,2-dimethyl-cyclopropane carboxylate (cypermethrin, CAS RN 52315-07-8), (beta-cypermethrin, CAS RN 65731-84-2), (theta cypermethrin, CAS RN 71697-59-1), S-cyano (3-phenoxyphenyl) methyl (±) cis/trans 3-(2,2-dichloroethenyl) 2,2 dimethylcyclopropane carboxylate (zeta-cypermethrin, CAS RN 52315-07-8), (s)-alpha-cyano-3-phenoxybenzyl (IR,3R)-3-(2,2-dibromovinyl)-2,2-dimethyl cyclopropanecarboxylate (deltamethrin, CAS RN 52918-63-5), alpha-cyano-3-phenoxybenzyl 2,2,3,3,-tetramethyl cyclopropoanecarboxylate (fenpropathrin, CAS RN 64257-84-7), (RS)-alpha-cyano-3-phenoxybenzyl(R)-2-[2-chloro-4-(trifluoromethyl)anilino]-3-methylbutanoate (tau-fluvalinate, CAS RN 102851-06-9), (2,3,5,6-tetrafluoro-4-methylpheny1)-methyl-(1 alpha, 3 alpha)-(Z)-(±)-3-(2-chloro-3,3,3-trifluoro-1-propeny1)-2,2-dimethylcyclopropane carboxylate (tefluthrin, CAS RN 79538-32-2), (±)-cyano (3-phenoxyphenyl) methyl (±)-4-(difluoromethoxy)-alpha-(1-methyl ethyl) benzeneacetate (flucythrinate, CAS RN 70124-77-5), cyano(4-fluoro-3-phenoxyphenyl)methyl3-[2-chloro-2-(4-chlorophenypetheny1]-2,2-dimethylcyclopropanecarboxylate (flumethrin, CAS RN 69770-45-2), cyano(4-fluoro-3-phenoxyphenyl)methyl 3-(2,2-dichloroethenyl)-2,2-dimethyl-cyclopropanedarboxylate (cyfluthrin, CAS RN 68359-37-5), (beta cyfluthrin, CAS RN 68359-37-5), (transfluthrin, CAS RN 118712-89-3), (S)-alpha-cyano-3-phenoxybenzyl Z)-(IR-cis)-2,2-dimethy1-3-[242,2,2-trifluoro-trifluoromethyl-ethoxycarbonyl)vinyl]cyclopropane carboxylate (acrinathrin, CAS RN 101007-06-1), (IR cis) S and (IS cis) R enantiomer isomer pair of alpha-cyano-3-phenoxybenzy1-3-(2,2dichloroviny1)-2,2-dimethylcyclopropane carboxylate (alpha-cypermethrin, CAS RN 67375-30-8), [IR,3 S)3 (1′RS)(1′,2′,2′,2′-tetrabromo ethyl)]-2,2-dimethyl cyclopropanecarboxylic acid(s)-alpha-cyano-3-phenoxybenzyl ester (tralomethrin, CAS RN 66841-25-6), cyano-(3-phenoxyphenyl) methyl 2,2-dichloro-1-(4-ethoxyphenyl)cyclopropane carboxylate (cycloprothrin, CAS RN 63935-38-6), [la, 3a(Z)]-(±)-cyano-(3-phenoxyphenyl)methyl 3-(2-chloro-3,3,3-trifluoro-1-propeny1)-2,2-cimethylcyc loprop anecarboxylate (cyhalothrin, CAS RN 68085-85-8), [1 alpha(s), 3 alp ha(z)]-cyano(3-pheno xyphenyl) methy1-3-(2-chloro-3,3,3-trifluoro-l-propeny1)-2,2-dimethylcyclopropane carboxylate (lambda cyhalothrin, CAS RN 91465-08-6), (2-methyl [1,1′-biphenyl]-3-y1) methyl 3-(2-chloro-3,3,3-trifluoro-1-prop eny1)-2,2-dimethyl-cyclopropanecarboxylate (bifenthrin, CAS RN 82657-04-3), 5-1-benzy1-3-furylmethyl-d-cis(1R,3S,E)2,2-dimethy1-3-(2-oxo,-2,2,4,5tetrahydro thiophenylidenemethyl)cyclopropane carboxylate (kadethrin, RU15525, CAS RN 58769-20-3), [5-(phenyl methyl)-3-furanyl]-3-furanyl 2,2-dimethyl-3-(2-methyl-l-propenyl) cyclopropane carboxylate (resmethrin, CAS RN 10453-86-8), (1R-trans)-[5-(phenylmethyl)-3-furanyl] methyl 2,2-dimethy1-3-(2-methyl-l-propenyl)cyclopropanecarboxylate (bioresmethrin, CAS RN 28434-01-7), 3,4,5,6-tetra hydro-phthalimidomethyl-(IRS)-cis-trans-chrysanthemate (tetramethrin, CAS RN 7696-12-0), 3-phenoxybenzyl-d,l-cis,trans 2,2-dimethy1-3-(2-methylpropenyl) cyclopropane carboxylate (phenothrin, CAS RN 26002-80-2); (empenthrin, CAS RN 54406-48-3); (cyphenothrin; CAS RN 39515-40-7), (prallethrin, CAS RN 23031-36-9), (imiprothrin, CAS RN 72963-72-5), (RS)-3-ally1-2-methyl-4-o xcyc lop ent-2-enyl-(1A,3R; 1R,3S)-2,2-dimethy1-3-(2-methylprop-1-enyl) cyclopropane carboxylate (allethrin, CAS RN 584-79-2), (bioallethrin, CAS RN 584-79-2), and (ZXI8901, CAS RN 160791-64-0). It is believed that mixtures of one or more of the aforementioned synthetic pyrethroids can also be used in the present invention. Particularly preferred synthetic pyrethroids are tefluthrin, lambda cyhalothrin, bifenthrin, permethrin and cyfluthrin. Even more preferred synthetic pyrethroids are tefluthrin and lambda cyhalothrin, and yet more preferred is tefluthrin.

Insecticides that are oxadiazine derivatives are useful in the subject invention. The oxadizine derivatives that are preferred for use in the present invention are those that are identified in U.S. Pat. No. 5,852,012. More preferred oxadiazine derivatives are 542-chloropyrid-5-ylmethyl)-3-methyl-4-nitro iminop erhydro-1,3,5-o xadiazine, 5-(2-chlorothiazol-5-ylmethyl)-3-methy1-4-nitro iminop erhydro-1,3,5-oxadiazine, 3-methyl-4-nitro imino-5-(1-o xido-3-pyridino methyl) p erhydro-1,3,5-o xadiazine, 5-(2-chloro-1-o xido-5-pyridinio methyl)-3-methyl-4-nitro iminop erhydro-1,3,5-o xidiazine; and 3-methyl-5-(2-methylpyrid-5-ylmethyl)-4-nitroiminoperhydro-1,3,5-oxadiazine. Even more preferred is thiamethoxam (CAS RN 15371923-4)

Chloronicotinyl insecticides are also useful in the subject invention. Chloronicotinyls that are preferred for use in the subject composition are described in U.S. Pat. No. 5,952,358, and include acetamiprid ((E)-N-[(6-chloro-3-pyridinyl)methyl]-N′-cyano-N-methyleneimidamide, CAS RN 135410-20-7), imidacloprid (1-[(6-chloro-3-pyridinyl)methol]-N-nitro-2-imidazolidinimime, CAS RN 138261-41-3), and nitenpyram (N-[(6-chloro-3-pyridinyl)methy1]-N-ethyl-N′-methy1-2-nitro-1,1-ethenediamine, CAS RN 120738-89-8).

Nitroguanidine insecticides are useful in the present invention. Such nitroguanidines can include those described in U.S. Pat. Nos. 5,633,375, 5,034,404 and 5,245,040.

Pyrrols, pyrazoles and phenyl pyrazoles that are useful in the present invention include those that are described in U.S. Pat. No. 5,952,358. Preferred pyrazoles include chlorfenapyr (4-bro mo-2-(4-chloropheny1)-1-etho xymethy1-5-trifluoromethylpyrro le-3-carbonitrile, CAS RN 122453-73-0), fenpyroximate ((E)-1,1-dimethylethy1-4[[[[(1,3-dimethy1-5-phenoxy-1H-pyrazole-4-yl)methylene]amino]oxy]methyl]benzoate, CAS RN 111812-58-9), and tebufenpyrad (4-chloro-N[[4-1,1-dimethylethyl)p henyl] methyl]-3-ethyl-l-methy1-1H-pyrazole-5-carboxamide, CAS RN 119168-77-3). A preferred phenyl pyrazole is fipronil (5-amino-[2,6-dichloro-4-(trifluoromethyl) phenyl]-4-[(1R, S)-(trifluoromethyl)sulflnyl]-1H-pyrazo le-3-carbonitrile, CAS RN 120068-37-3).

Diacylhydrazines that are useful in the present invention include halofenozide (4-chlorobenzoate-2-benzoyl-2-(1,1-dimethylethyl)-hydrazide, CAS RN 112226-61-6), methoxyfenozide (RH-2485; N-tert-butyl-N′-(3-methoxy-o-toluoy1)-3,5-xylohydrazide, CAS RN 161050-58-4), and tebufenozide (3,5-dimethylbenzoic acid 1-(1,1-dimethylethyl)-2, (4-ethylbenzoyphydrazide, CAS RN 112410-23-8).

Triazoles, such as amitrole (CAS RN 61-82-5) and triazamate are useful in the nethod of the present invention. A preferred triazole is triazamate (ethyl[[1-[(dimethylamino)carbonyl]-3-(1,1-dimethylethyl)-1H-1,2,4-triazol-5-yl]thio] acetate, CAS RN 112143-82-5).

Biological/fermentation products, such as avermectin (abamectin, CAS RN 71751-41-2) and spinosad (XDE-105, CAS RN 131929-60-7) are useful in the present invention.

Organophosphate insecticides are also useful as one of the components of the present invention. Preferred organophophate insecticides include acephate (CAS RN 30560-19-1), chlorpyrifos (CAS RN 2921-88-2), chlorpyrifos-methyl (CAS RN 5598-13-0), diazinon (CAS RN 333-41-5), fenamiphos (CAS RN 22224-92-6), and malathion (CAS RN 121-75-5).

In addition, carbamate insecticides are useful in the subject invention. Preferred carbamate insecticides are aldicarb (CAS RN 116-06-3), carbaryl (CAS RN 63-25-2), carbofuran (CAS RN 1563-66-2), oxamyl (CAS RN 23135-22-0) and thiodicarb (CAS RN 59669-26-0).

When a chemical insecticide is described herein, it is to be understood that the description is intended to include salt forms of the insecticide as well as any isomeric and/or tautomeric form of the insecticide that exhibits the same insecticidal activity as the form of the insecticide that is described.

The chemical insecticides that are useful in the present invention can be of any grade or purity that pass in the trade as such insecticide. Other materials that accompany the insecticides in commercial preparations as impurities can be tolerated in the subject invention and compositions, as long as such other materials do not destabilize the composition or significantly reduce or destroy the activity of any of the insecticide components or the transgenic event against the target pest(s). One of ordinary skill in the art of the production of insecticides can readily identify those impurities that can be tolerated and those that cannot.

eHTP's are related by amino acid modifications such that the modified proteins exhibit enhanced Hemipteran inhibitory spectrum and/or improved Hemipteran inhibitory activity against Lygus spp., Empoasca spp. and/or Amrasca spp. compared to the parent protein, TIC807. The phrases “more active”, “improved activity”, “enhanced specificity”, “increased toxic potency”, “increased toxicity”, “improved Hemipteran inhibitory activity, “enhanced Hemipteran inhibitory activity”, “improved Lygus, Empoasca and/or Amrasca inhibitory activity”, “greater Lygus, Empoasca and/or Amrasca inhibitory activity”, “greater Hemipteran inhibitory activity” and “enhanced Lygus, Empoasca and/or Amrasca inhibitory spectrum” and “enhanced Hemipteran inhibitory spectrum” refer to a comparison of the activity of an eHTP and of the activity of a TIC807 (SEQ ID NO:2), TIC807 M2 (SEQ ID NO:8), Cry51Aa1 (SEQ ID NO:182), TIC853 (SEQ ID NO:184), and/or a AXMI-171 (SEQ ID NO:206) protein against a Hemipteran insect, wherein activity attributed by the eHTP of the present invention is greater than the activity attributed to the TIC807 protein (SEQ ID NO:2), TIC807 M2 (SEQ ID NO:8), Cry51Aa1 (SEQ ID NO:182), TIC853 (SEQ ID NO: 184, and/or a AXMI-171 (SEQ ID NO:206) protein. eHTP's provided herein exhibit enhanced Hemipteran inhibitory spectrum and/or improved or greater Hemipteran inhibitory activity when compared to the Bacillus thuringiensis proteins of SEQ ID NO:2, SEQ ID NO:8, SEQ ID NO:182, and SEQ ID NO: 184, where the Hemipteran pest species include Lygus hesperus, Lygus lineolaris, Empoasca fabae, and Amrasca devastans. Amrasca devastans is also called Amrasca biguttula biguttula. eHTP's exhibiting enhanced insect inhibitory spectrum and/or improved insect inhibitory activity compared to TIC807 can be identified by many different methods. In general, exemplary and non-limiting methods for identifying eHTP proteins can comprise:
(1) administering identical amounts of a test eHTP and of control TIC807 (SEQ ID NO:2), TIC807 M2 (SEQ ID NO:8), Cry51Aa1 (SEQ ID NO:182), TIC853 (SEQ ID NO:184), and/or an AXMI-171 (SEQ ID NO:206) protein to a test insect under controlled assay conditions; and, measuring and comparing the potency of the test and control proteins; and/or,
(2) determining the protein doses (e.g., protein concentration in diet) of a test eHTP and of control TIC807 (SEQ-ID NO: 2), TIC807 M2 (SEQ ID NO:8), Cry51Aa1 (SEQ ID NO:182), TIC853 (SEQ ID NO:184), and/or an AXMI-171 (SEQ ID NO:206) protein which elicit equivalent insect population responses under controlled assay conditions (i.e. obtaining a dose response curve).
   In the second approach, a statistically robust dose response value used for comparison would be the median lethal concentration (LC50) required to kill 50% of a test population. However, in certain embodiments, other values including but not limited to, a median inhibitory concentration (“IC50”) required to result in 50% growth inhibition of a test population can be used. In this context, “growth inhibition” can comprise stunting and/or inhibition of Hemipteran development.

As used herein, the phrase “an insect inhibitory amount”, refers to an amount of a composition containing an agent that is effective in achieving any measurable inhibition of insect viability, growth, insect development, insect reproduction, insect feeding behavior, insect mating behavior and/or any measurable decrease in the adverse effects caused by insect feeding on a composition containing the agent. Similarly, a “Hemipteran inhibitory amount” refers to an amount of a protein of the present invention alone or with other agents targeting the applicable Hemipteran species for control, that results in any measurable inhibition of target insects belonging to the order Hemiptera related to viability, growth, development, reproduction, feeding behavior, mating behavior, and or any measurable decrease in the adverse effects caused by Hemipteran insects feeding on a plant. Likewise, “Lygus, Empoasca and/or Amrasca inhibitory amount” refers to an amount of a composition containing one or more proteins of the present invention, i.e., eHTP's, or other agent that results in any measurable inhibition, viability, growth, development, reproduction, feeding behavior, mating behavior and/or any measurable decrease in the adverse effects caused by Lygus, Empoasca and/or Amrasca feeding on a composition containing that eHTP. As used herein in the context of an eHTP, an “enhanced Hemipteran inhibitory activity or “greater enhanced Hemipteran inhibitory activity” refers to any measurable increase in the inhibition of Hemipteran viability, growth, development, reproduction, feeding behavior, mating behavior and/or any measurable decrease in the adverse effects caused by Hemipteran feeding on a composition containing that eHTP relative to the corresponding inhibitory activity observed with any one or more of the scaffold proteins, including TIC807, Cry51Aa1 (SEQ ID NO:182), TIC853 (SEQ ID NO:184), and/or AXMI-171 (SEQ ID NO:206) proteins. Likewise, “enhanced Lygus, Empoasca and/or Amrasca inhibitory activity” or “greater enhanced Lygus, Empoasca and/or Amrasca inhibitory activity” refers to any measurable increase in the inhibition, viability, growth, development, reproduction, feeding behavior, mating behavior and/or any measurable decrease in the adverse effects caused by the presence of one or more eHTP of the present invention in a composition or plant provided in the diet of Lygus, Empoasca and/or Amrasca relative to the corresponding inhibitory activity observed with an equivalent composition or plant containing only an applicable amount of one or more of the scaffold proteins, including but not limited to TIC807 (SEQ ID NO:2), Cry51Aa1 (SEQ ID NO: 182), TIC853 (SEQ ID NO:184), and/or AXMI-171 (SEQ ID NO:206) proteins.

As used herein in the context of an eHTP, an “enhanced Lygus, Empoasca and/or Amrasca inhibitory spectrum” refers to any measurable increase in the inhibition of a specific Lygus spp., Empoasca spp. and/or Amrasca spp. viability, growth, development, reproduction, feeding behavior, mating behavior and/or any measurable decrease in the adverse effects caused by that Lygus spp., Empoasca spp. and/or Amrasca spp. feeding on a plant relative to the corresponding inhibition of that specific Lygus spp., Empoasca spp. and/or Amrasca spp. observed with the TIC807 protein. In certain embodiments, eHTP provided herein exhibit an enhanced Lygus inhibitory spectrum relative to TIC807 in that those eHTP's can provide increased inhibition of Lygus lineolaris.

An eHTP provided herein can exhibit from about 2 to about 260 fold greater Lygus, Empoasca and/or Amrasca inhibitory activity against a Lygus, Empoasca and/or Amrasca pest species than a protein of SEQ ID NO:2 (TIC807), SEQ ID NO:8 (TIC807 M2), SEQ ID NO:182 (Cry51Aa1), SEQ ID NO:184 (TIC853), and SEQ ID NO:206 (AXMI-171). An eHTP provided herein can exhibit from about 3, 4, 5, 7, 8, 10, 12, 15, 20, 25, 27, 30, 38, 46, 50, 52, 54, 66, 91, 122, 186, 243, or 262 fold greater Lygus, Empoasca and/or Amrasca inhibitory activity against a Lygus, Empoasca and/or Amrasca pest species than a protein of SEQ ID NO:2 (TIC807), SEQ ID NO:8 (TIC807 M2), SEQ ID NO:182 (Cry51Aa1), SEQ ID NO:184 (TIC853), and SEQ ID NO:206 (AXMI-171).

eHTP's can exhibit an enhanced target pest inhibitory spectrum and/or improved target pest inhibitory activity over SEQ ID NO:2 (TIC807), SEQ ID NO:8 (TIC807 M2), SEQ ID NO:182 (Cry51Aa1), SEQ ID NO:184 (TIC853), and/or a SEQ ID NO:206 (AXMI-171) by causing mortality:
(i) at a dose of about 0.3 μg/mL to about 70 μg/mL against a Lygus hesperus insect species,
(ii) at a dose of about 0.85 μg/mL to about 100 μg/mL against a Lygus lineolaris insect species,
(iii) measuring at an LC50 value of about 0.3 to about 70 μg/mL against Lygus hesperus,
(iv) measuring at an LC50 value of about 0.85 to about 100 μg/mL against Lygus lineolaris, or
(v) measuring at an LC50 value of more than two-fold lower the LC50 value of TIC807, SEQ ID NO: 8, SEQ ID NO:182 (Cry51Aa1), SEQ ID NO:184 (TIC853), and/or a SEQ ID NO: 206 (AXMI-171) against Lygus spp, Emrasca spp. and/or Amrasca spp., or
(vi) at a dose of about 0.69 μg/mL to about 500 μg/mL against a Amrasca devastans or Empoasca fabae insect species, or
(vii) measuring at an LC50 value of about 3.5 to about 15 μg/mL against Amrasca devastans and/or Empoasca fabae.

Table 4A and 4B tabulate the exemplary eHTP's of the present invention with Amrasca and Lygus spp. mortality data. Mortality data available for Lygus spp. and Amrasca spp. are reported either as (a) a μg/mL LC50 value, or as (b) a % mortality at doses of about 1 to about 3 μg/mL for L. hesperus or about 100 μg/mL protein for L. lineolaris, and about 0.69 to 500 μg/mL for Amrasca devastans. The fold increased toxicity compared to TIC807 (SEQ ID NO:2) and TIC807 M2 (SEQ ID NO:8) is provided for exemplary eHTP's where LC50 values were determined.

The eHTP's of the present invention are particularly useful in controlling insects of the order Hemiptera compared to the scaffold proteins. Lygus lineolaris required high doses of TIC807 protein (e.g., in excess of 100 μg/mL) to elicit mortality. The dose response curve for one eHTP of the present invention TIC807 M8 (SEQ ID NO:16), an eHTP that exhibits remarkably improved toxic effects against both L. lineolaris and L. hesperus, but against L. lineolaris the eHTP exhibits a calculated LC50 value of 223 μg/mL. It has not been possible previously to achieve a protein concentration toxic dose that can elicit greater than 50% mortality against L. lineolaris species because providing significantly large doses of TIC807 and TIC807 M2 protein in excess of 1000 μg/mL in the diet has not been possible. Therefore, LC50 values against L. lineolaris for TIC807 and TIC807 M2 (SEQ ID NO:8) proteins were not determined, but rather estimated as greater than (>) 223 μg/mL (See Tables 1 and 3, Example 4, and FIG. 1B).

Iterative design refers to a semi-random approach for developing and selecting eHTP's including a combination of engineering, testing, and selecting (not necessarily in that order) (see Examples 1 through 4). The word “engineering” is intended to include identifying relevant residues to modify, cloning, and expressing eHTP's described herein. The word “testing” is intended to refer to comparing the Hemipteran activity of an eHTP to the activity of a scaffold protein such as TIC807 (SEQ ID NO:2), TIC807 M2 (SEQ ID NO: 8), Cry51Aa1 (SEQ ID NO:182), and/or TIC853 (SEQ ID NO:184); or, comparing an eHTP of the present invention against another protein such as AXMI-171 (SEQ ID NO:206). The word “selecting” is intended to refer to the act of identifying improved variant proteins of the present invention, i.e., eHTP's, and the applicable amino acid residues for “engineering”.

Iterative design includes the elucidation of the atomic structure of proteins of the present invention (for example, as set forth in FIG. 2) and the use of the atomic structure to guide and complement semi-random approaches of “selecting” amino acid residues to modify for “engineering”, and in this case, has included the identification of amino acid residues at loops and at surface exposed regions of a folded insect inhibitory scaffold protein such as TIC807, TIC853, and Cry51Aa1 that can be modified to confer improvements to insect inhibitory spectrum and activity. Such amino acid residues at loops and at surface exposed regions are selected for “engineering”. In this case, iterative design has included the identification of two different regions within the three dimensional structure of the scaffold protein that harbor an accumulation of relevant amino acid residues that, when modified to contain amino acid residues other than those appearing at those positions in the naturally occurring scaffold protein, result in one or more of the eHTP proteins of the present invention.

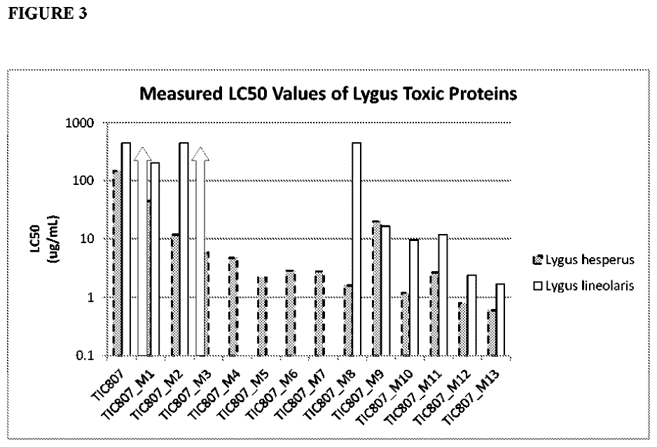

Initially the scaffold protein TIC807 (SEQ ID NO:2) used in this process of iterative design, and 267 different eHTP's were discovered that exhibited increased Lygus spp. inhibitory activity compared to the scaffold protein TIC807. TIC807 M8 (SEQ ID NO:16) was discovered in early rounds of the design process. Subsequent rounds of iterative engineering-testing-selecting led to the discovery of other eHTP proteins that exhibited yet greater levels of toxicity against Lygus species and also exhibited a broader host range of toxic effects when compared to the scaffold protein. Seven variants (eHTP's) exhibited significantly higher levels of increased toxicity against both Lygus species (L. hesperus and L. lineolaris) when compared to TIC807. LC50 values for these seven, and other, eHTP's constructed herein were determined against Lygus hesperus and Lygus lineolaris species and compared to LC50 values for scaffold proteins, particularly TIC807. The results are shown in Table 1, and FIG. 3 is a bar chart showing graphically the results observed as tabulated in Table 1.

TABLE 1
LC50 values of select eHTP's compared to TIC807

| | | Lygus hesperus | | Lygus lineolaris | |
|---|---|---|---|---|---|
| SEQ | | LC50 | Toxicity | LC50 | Toxicity |
| ID | | value' | (fold | value | (fold |
| NO: | Toxin | (μg/mL) | increase) | (μg/mL) | increase) |
| 2 | TIC807 | 73 | 1 | >223* | 1 |
| 6 | TIC807_M1 | 23 | 3 | 100 | >2 |
| 8 | TIC807_M2 | 5.9 | 12 | >223* | −1 |
| 10 | TIC807_M3 | 2.9 | 25 | ND | — |
| 12 | TIC807_M4 | 2.4 | 30 | ND | — |
| 14 | TIC807_M5 | 1.1 | 66 | ND | — |
| 18 | TIC807_M6 | 1.45 | 50 | ND | — |
| 20 | TIC807_M7 | 1.4 | 52 | ND | — |
| 16 | TIC807_M8 | 0.8 | 91 | 223 | >1 |
| 28 | TIC807_M9 | 9.9 | 7 | 8.3 | >27 |
| 30 | TIC807_M10 | 0.6 | 122 | 4.8 | >46 |
| 32 | TIC807_M11 | 1.35 | 54 | 5.9 | >38 |
| 36 | TIC807_M12 | 0.4 | 182 | 1.2 | >186 |
| 34 | TIC807_M13 | 0.3 | 243 | 0.85 | >262 |

ND = Not Determined.
LC50 values are determined by presenting 8-10 different protein concentrations to a population of newly hatched Lygus nymphs, allowing nymphs to feed for 5 days, and then scoring for mortality over the dose range provided.
*Toxicity, displayed in terms of a multiple of increased activity compared to the level observed against Lygus hesperus using the observed LD50 for TIC807 as the baseline value of 1. Significantly large amounts of protein in excess of 1000 [μg/mL have not been possible to provide in Lygus diet in order to complete the high range of toxicity dose response to Lygus lineolaris. Therefore, an LC50 value was not determined for TIC807 or TIC807_M2. Instead, a 4-dose LC50 estimation in the low range was performed verifying that expected LC50 values for TIC807 and TIC807_M2 are greater than 223 [μg/mL.

With reference to Table 1, the iterative design process has provided a means for identifying proteins exhibiting improved toxic properties, not only to Lygus hesperus, but also to Lygus lineolaris.

Recombinant polynucleotide compositions that encode eHTP's are also provided. In certain embodiments, eHTP's can be expressed with recombinant DNA constructs in which a polynucleotide molecule with the open reading frame encoding the protein is operably linked to elements such as a promoter and any other regulatory element functional for expression in the system for which the construct is intended. For example, plant-functional promoters can be operably linked to an applicable eHTP coding sequence to enable expression of the protein in plants. Promoters functional in bacteria are also contemplated for use in expression cassettes. Promoters functional in an applicable bacterium, for example, in an E. coli or in a Bacillus thuringiensis species can be operably linked to the eHTP coding sequences for expression of the applicable protein in the applicable bacterial strain. Other useful elements that can be operably linked to the eHTP coding sequences include, but are not limited to, enhancers, introns, leaders, encoded protein immobilization tags (HIS-tag), encoded sub-cellular translocation peptides (i.e. plastid transit peptides, signal peptides), encoded polypeptide sites for post-translational modifying enzymes, ribosomal binding sites, and segments designed for use as RNAi triggers for suppression of one or more genes either in plants or in a particular target pest species.

Exemplary recombinant polynucleotide molecules provided herein include, but are not limited to, SEQ ID NO:186, SEQ ID NO:187, SEQ ID NO:188, SEQ ID NO: 189, SEQ ID NO: 190, SEQ ID NO:191, SEQ ID NO: 192, SEQ ID NO:193, SEQ ID NO: 194, SEQ ID NO: 195, SEQ ID NO:196, SEQ ID NO:197, SEQ ID NO:198, SEQ ID NO:199, SEQ ID NO:200, SEQ ID NO:35, and SEQ ID NO:201. These sequences encode the respective proteins each having the amino acid sequence as set forth in SEQ ID NO:4 (TIC807 4), SEQ ID NO:6 (TIC807 M1), SEQ ID NO:8 (TIC807 M2), SEQ ID NO:10 (TIC807 M3), SEQ ID NO: 12 (TIC807 M4), SEQ ID NO:14 (TIC807 M5), SEQ ID NO:16 (TIC807 M8), SEQ ID NO:18 (TIC807 M6), SEQ ID NO:20 (TIC807 M7), SEQ ID NO:22 (TIC807 22), SEQ ID NO:24 (TIC807 24), SEQ ID NO:26 (TIC807 26), SEQ ID NO:28 (TIC807 M9), SEQ ID NO:30 (TIC807 M10), SEQ ID NO:32 (TIC807 M11), SEQ ID NO:36 (TIC807 M12), and SEQ ID NO:34 (TIC807 M13). Because of the redundancy of the genetic code, the codons of a recombinant polynucleotide molecule encoding for proteins of the present invention may be substituted for synonymous codons (also called a silent substitution); and are within the scope of the present invention. Recombinant polynucleotides encoding any of the eHTP's disclosed herein are thus provided.

A recombinant DNA construct comprising eHTP coding sequences can also further comprise a region of DNA that codes for one or more insect inhibitory agents which can be configured to be co-expressed along with a DNA sequence encoding an applicable eHTP, a protein different from an eHTP, or an insect or plant gene inhibitory dsRNA molecule. A recombinant DNA construct can be assembled so that all agents designed to be expressed from a particular construct are expressed from one promoter or so that separate agents are each under separate promoter control, or some combination thereof. The proteins of this invention can be expressed from a multi-gene expression system in which one or more proteins are expressed from a common nucleotide segment on which is also contained other open reading frames and/or promoters depending on the type of expression system selected.

Recombinant polynucleotide or recombinant DNA construct comprising an eHTP encoding sequence can be delivered to host cells by vectors, e.g., a plasmid, baculovirus, artificial chromosome, virion, cosmid, phagemid, phage, or viral vector. Such vectors can be used to achieve stable or transient expression of an eHTP encoding sequence in a host cell; and, if the case may be, subsequent expression to polypeptide. An exogenous recombinant polynucleotide or recombinant DNA construct that comprises an eHTP encoding sequence and that is introduced into a host cell is also referred to herein as a “transgene”.

Also provided herewith are transgenic bacteria, transgenic plant cells, transgenic plants, and transgenic plant parts that contain any a recombinant polynucleotide (i.e. transgene) that expresses any one or more eHTP encoding sequence. It is intended that “bacterial cell” or “bacterium” can include, but are not limited to, an Agrobacterium, a Bacillus, an Escherichia, a Salmonella, a Pseudomonas, or a Rhizobium cell. It is intended that “plant cell” or “plant” include an alfalfa, almont, banana, barley, bean, beet, broccoli, cabbage, brassica, brinjal, carrot, cassava, castor, cauliflower, celery, chickpea, Chinese cabbage, celery, citrus, coconut, coffee, corn, clover, cotton, a cucurbit, cucumber, Douglas fir, eggplant, eucalyptus, flax, garlic, grape, guar, hops, leek, legumes, lettuce, Loblolly pine, millets, melons, nectarine, nut, oat, okra, olive, onion, ornamental, palm, pasture grass, papaya, pea, peach, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell or plant. In certain embodiments: transgenic plants and transgenic plant parts regenerated from a transgenic plant cell are provided; transgenic plants can be obtained from a transgenic seed; transgenic plant parts can be obtained by cutting, snapping, grinding or otherwise disassociating the part from the plant; the plant part can be a seed, a boll, a leaf, a flower, a stem, a root, or any portion thereof; and a transgenic plant part provided herein is a non-regenerable portion of a transgenic plant part. As used in this context, a “non-regenerable” portion of a transgenic plant part is a portion that can not be induced to form a whole plant or that can not be induced to form a whole plant that is capable of sexual and/or asexual reproduction. A non-regenerable portion of a plant part is a portion of a transgenic pollen, ovule, seed, boll, leaf, flower, stem, or root.

Also provided herein are methods of making transgenic plants that contain insect or Lygus and/or Amrasca inhibitory amounts of an eHTP. Such plants can be made by introducing a recombinant polynucleotide that encodes any of the eHTP proteins provided herein into a plant cell, and selecting a plant derived from said plant cell that expresses an insect or Hemipteran inhibitory amount of the eHTP's. Plants can be derived from the plant cells by regeneration, seed, pollen, or meristem transformation techniques.

Transgenic plants and host cells are provided that expresse an insect or Hemipteran inhibitory amount of the eHTP to control an insect or Hemipteran infestation. Any of the aforementioned plant species can be used for protecting a plant from insect or Hemipteran infestation provided herein as long as the plant is transformed with a polynucleotide construct designed to express the applicable eHTP.

Additional aspects of the invention include antibodies, kits, methods for detecting polynucleotides that encode eHTP's or distinguishing fragments thereof, or eHTP's or distinguishing fragments thereof, methods for identifying additional insect inhibitory members of the protein genus of the present invention, formulations and methods for controlling insect growth and/or infestation, and methods for providing such control to plants and other recipient hosts. Each composition, construct, cell, plant, formulation, method or kit provides for the industrial application of the proteins of the present invention, for example, by increasing plant productivity through the commercial use of any of these proteins to inhibit insects.

A plant product, other than a seed or a fruit or vegetable, is intended as a commodity or other products which move through commerce and are derived from a transgenic plant or transgenic plant part, in which the commodity or other products can be tracked through commerce by detecting nucleotide segments, RNA or proteins that corresponding to an eHTP of the present invention and are produced in or maintained in the plant or plant tissue or part from which the commodity or other product has been obtained. Such commodity or other products of commerce include, but are not limited to, plant parts, biomass, oil, meal, sugar, animal feed, flour, flakes, bran, lint, processed seed, and seed. Plant parts include but are not limited to a plant seed, boll, leaf, flower, stem, pollen, or root. In certain embodiments, the plant part is a non-regenerable portion of said seed, boll, leaf, flower, stem, pollen, or root. Cotton and flax plant bolls and non-regenerable portions thereof that contain the eHTP's are also provided.

Also provided herewith are processed plant products that contain a detectable amount of an eHTP, an insect inhibitory fragment thereof, or any distinguishing portion thereof. Without seeking to be limited by theory, it is believed that such processed plant products containing a detectable amount of one or more of the eHTP's provided herein can in certain embodiments exhibit reductions in undesirable microorganisms that can be transmitted by Hemiptera and/or reductions in the undesirable side products of such microorganisms. In certain embodiments, a distinguishing portion thereof can comprise any polypeptide of at least from about 20 to about 100 or more contiguous amino acids as set forth in SEQ ID NO:180, in particular in which the polypeptide does not contain a corresponding polypeptide of contiguous amino acids present in SEQ ID NO:2, SEQ ID NO:8, SEQ ID NO:182, or SEQ ID NO: 184, and wherein the polypeptide comprises at least one amino acid substitution, addition, or deletion in the corresponding amino acid sequence as set forth in SEQ ID NO:2. Such substitutions, deletions or additions are those as set forth above in paragraph [0009].

Processed plant products are provided that contain a detectable amount of a recombinant polynucleotide encoding an eHTP, an eHTP or an insect inhibitory fragment thereof, or any distinguishing portion thereof. The processed product is selected from the group consisting of plant biomass, oil, meal, animal feed, flour, flakes, bran, lint, hulls, and processed seed.

Hemiptera infestations of crop plants are controlled by providing in the crop plants a recombinant polynucleotide sequence encoding one or more of the eHTP's of the present invention. Such transgenic crops produce or are treated to contain an insect or Hemiptera inhibitory amount of an applicable eHTP, and such crops are imbued with sufficient eHTP by (i) applying any composition comprising or encoding an eHTP to the plant or a seed that gives rise to the plant; and/or (ii) transforming the plant or a plant cell that gives rise to the seed and ultimately, the plant, with a polynucleotide encoding an eHTP. The plant may be a transiently or stably transformed transgenic plant comprising a transgene that expresses an insect or Hemiptera inhibitory amount of an eHTP. The plant may be a non-transgenic plant to which a composition comprising an eHTP has been applied. In such methods, the plant is a dicot plant, and more specifically may be a cotton, soybean or alfalfa plant. The Hemipteran insects include adults and nymphs, such as but not limited to the listing of bugs that is set forth above in paragraph [0020].

Preferably, the Lygus spp. is Lygus hesperus or Lygus lineolaris, the Empoasca spp. is Empoasca fabae, and the Amrasca spp. is Amrasca devastans.

Other features and advantages of the invention will be apparent from the following detailed description, examples, and claims.

### EXAMPLES

In view of the foregoing, those of skill in the art should appreciate that changes can be made in the specific aspects which are disclosed and still obtain a like or similar result without departing from the spirit and scope of the invention. Thus, specific details disclosed herein are not to be interpreted as limiting. The U.S. Provisional Application Ser. No. 61/621,436 to which this application claims the benefit of priority, the Sequence Listing referenced in paragraph [0002], as well as all references material to the inventions disclosed and claimed, particularly references and published patent applications cited in this application, are incorporated herein by reference in their entirety.

### Example 1: Iterative Engineering-Testing-Selecting Approach

This example illustrates the random, combinatorial, and inventive aspects of the iterative (also can be referred to as “recursive”) engineering-testing-selecting approach used to identify and describe insect inhibitory proteins exhibiting Coleopteran and/or nematicidal activity or increased toxicity to Hemipteran insect species compared to TIC807 (SEQ ID NO:2). Several design approaches were employed to engineer for eHTP's with greater inhibitory activity against Lygus species; approaches that included but were not limited to semi-random modifications, directed modifications of variances in an alignment of TIC807 with other native Bt proteins, and structure/function assisted design. Numerous rounds of engineering and testing were conducted (both consecutively and concurrently) to select for TIC807 protein variants exhibiting increased toxicity. Design approaches were adjusted as data was collected. This iterative engineering-testing-selection approach also included, but was not limited to steps including cloning, expressing, purifying, and bioassay testing of TIC807 control protein compared to the eHTP's.

About 267 exemplary eHTP's having exhibited increased Lygus toxicity compared to TIC807 were obtained from more than 2000 groups of candidate eHTP's (i.e. “test” proteins) that were assayed for improved insect inhibitory activity. The actual total number of candidate eHTP's tested was much greater than 2000 because testing included recombinant nucleotide segments encoding a number of candidate eHTP's derived from library mutagenesis that were not sequenced in the selection process.

Protein stocks of various amounts and purity were prepared depending on the purpose of the test and the testing throughput desired. For example, lower quantity and lower purity protein preparations were prepared for screening higher numbers of variants in bioassay. Larger quantity and higher purity protein stocks were prepared for high-powered bioassays. Testing trended towards the high-powered bioassays as principally relevant residue positions of the improved variants were elucidated. Initially, about 2000 variants were tested on Lygus hesperus. Based on data from L. hesperus approximately 600 variants were designed and then further tested on Lygus lineolaris. Of these, about 267 variants (Table 4B) demonstrated increased toxicity against Lygus hesperus and/or Lygus lineolaris when compared to TIC807. These 267 variants included twenty-two (22) variants that were confirmed to demonstrate increased toxicity against both Lygus species. Further confirmation and dose response testing narrowed the selection to seven (7) variants that were subsequently characterized using an 8-dose replicated bioassay to determine LC50 values against both Lygus species.

The selection process included dynamic updates of testing data, constantly adjusting engineering approaches, and performing iterative rounds. Concurrently, labor intensive cloning, protein expression, protein purification, and bioassay experiments were employed test the candidate eHTP's.

### Example 2: Engineering Approaches

### Alignment Based Approaches

A multiple sequence alignment of protein members of Cry51: Cry51Aa1 (SEQ ID NO:182), TIC853 (SEQ ID NO:184), and TIC807 (SEQ ID NO:2) were used to identify regions of variability, e.g., positions 195 to 201 and positions 211 to 219, relative to SEQ ID NO:2 (TIC807). These regions were targeted for saturation mutagenesis through use of degenerate oligonucleotide primers encoding random amino acid residues in these regions. Construct libraries were prepared for subsequent protein expression in host cells.

A multiple sequence alignment of Cry51Aa1 (SEQ ID NO:182), TIC853 (SEQ ID NO:184), and TIC807 (SEQ ID NO:2) was used in combination with a BLOSUM 80 substitution matrix to calculate average pair-wise distances for each position variant to TIC807. Residue positions with lower average pair-wise distances were substituted with alternative amino acid residues using degenerate oligonucleotide primers encoding for alternative amino acid residues, e.g., G28X, G31X, F46X, E125X, F138X, F147X, S167X, Y216X, P218X, G234X, T247X, D268X, and T308X. Construct libraries were prepared for subsequent protein expression in host cells.

### Scanning Approaches

Polynucleotide constructs were engineered to express a single Alanine substitution or a double Alanine substitution (Alanine-<parent residue>-Alanine) at every possible position over the full-length of SEQ ID NO:2 (TIC807). See Table 2 for a hypothetical example.

TABLE 2
A hypothetical example of single and double
Alanine scans on a scaffold protein containing
the amino acid sequence XXXXAXX.

| | Single Alanine Scan | Double Alanine Scan |
|---|---|---|
| 1 | AXXXaXX | AXAXaXX |
| 2 | XAXXaXX | XAXAaXX |
| 3 | XXAXaXX | XXAXSXX |
| 4 | XXXAaXX | XXXAaAX |
| 5 | XXXXSXX | XXXXSXA |
| 6 | XXXXaAX | — |
| 7 | XXXXaXA | — |

X = parent residue
a = parent residue is an Alanine residue
A = Modified to an Alanine residue
S = Modified to a Serine residue

Where an Alanine residue was already present in TIC807, a Serine was substituted instead. Protein variants that exhibited increased toxicity compared to TIC807 were further tested by combination and saturation mutagenesis at those Alanine-substituted residues that conferred increased toxicity. Scanning approaches were also performed on improved combination variants having accumulated modifications from previous iterative rounds of engineering-testing-selecting, e. g., TIC807 M2 (SEQ ID NO:8) having mutations F46S, Y54H, S167R, S217N, and a contiguous triple deletion in residue range 196-201 was further engineered by an additional round of single Alanine substitutions to further improve upon the improved TIC807 M2. Principally relevant residues were identified and further tested by combination and saturation mutagenesis (e. g., A150X, E125X, E155X, F147X, 1134X, N157X, Q149X, T133X, E135X, and N137X). Variants engineered by these combined approaches exhibited further improvements to increased toxicity compared to TIC807 and were further combined with other design approaches that took advantage of the atomic structure of TIC807 (SEQ ID NO:2).

### Surface Exposed Residues

The atomic structure of proteins of the present invention was determined in the midst of the Iterative Engineering-Testing-Selecting approach; and, the relative solvent-accessibility (% SA) of each residue was determined using Molsoft's ICM-Browser (Molsoft L.L.C., 11199 Sorrento Valley Road, 5209, San Diego, CA 92121). Shown in Table 3 in columns (A) and (B), actual % SA was calculated for proteins having the respective amino acid sequences set forth as SEQ ID NO:185 (TIC807 L11M) and SEQ ID NO:8 (TIC807 M2). The predicted % SA for residues of TIC807 and TIC853 are listed in Table 3 in columns (A) and (C), respectively. Altogether, the % SA values reported in Table 3 are calculated as a percentage of the solvent-accessible surface area probed by a water molecule over the maximal solvent accessible area in standard extended conformation (Gly-XXX-Gly) for each residue in each position of the atomic structure. Table 3 aligns the residues of each protein by aligned residues in a Clustal W alignment. % SA greater than 100 can occur when maximal solvent accessible area in standard extended conformation (Gly-XXX-Gly) for each residue is less than the actual solvent accessible area probed by a water molecule. % SA greater than 100 are reported in the table as 100%.

Combined engineering-testing-selecting approaches described herein resulted in a number of principally relevant residues that accumulate in a surface patch ([2] of FIG. 2) of residues having a radius of about 9.2-12.2 Angstroms around the Cb atom of P219 of SEQ ID NO:2 (TIC807): V10, 114, N22, N23, G24, 125, Q26, G27, F30, Q38, 139, D40, T41, 143, S193, T194, E195, H196, Y197, S198, H199, Y200, S201, G202, Y203, P204, 1205, L206, T207, W208, 1209, S210, Y216, S217, G218, P219, F220, M221, S222, W223, Y224, F225, N239, and V244 of SEQ ID NO:2 (TIC807). At least half of these residues exhibit % SA values of greater or equal to fifteen (15).

TABLE 3
Relative % Solvent Accessability (SA) of
Amino Acids of eHTP's & Scaffold Proteins.

| (A) | | (B) | | (C) | |
|---|---|---|---|---|---|
| TIC807_L11M | | TIC807_M2 | | TIC853 | |
| (SEQ ID NO: 185) | | (SEQ ID NO: 8) | | (SEQ ID NO: 184) | |
| Position | | Position | | Position | |
| and amino | Calculated | and amino | Calculated | and amino | Estimated |
| acid | % SA per | acid | % SA per | acid | % SA per |
| residue | residue | residue | residue | residue | residue |
| 2ALA | 82.1# | 2ALA | 64.8# | 2ALA | 60.9# |
| 3ILE | 23.3 | 3ILE | 28.9 | 3ILE | 24.3 |
| 4LEU | 26.4 | 4LEU | 31.9 | 4LEU | 27.9 |
| 5ASP | 26.0 | 5ASP | 22.7 | 5ASP | 29.9 |
| 6LEU | 1.0 | 6LEU | 3.4 | 6LEU | 3.7 |
| 7LYS | 25.1 | 7LYS | 16.2 | 7LYS | 17.1 |
| 8SER | 46.4# | 8SER | 37.6# | 8SER | 44.9# |
| 9LEU | 8.9 | 9LEU | 5.2 | 9LEU | 6.3 |
| 10VALP¹ | 0.3 | 10VAL | 0.6 | 10VAL | 0.0 |
| 11MET | 25.0 | 11LEU | 17.0 | 11LEU | 16.6 |
| 12ASN* | 49.8# | 12ASN | 43.2# | 12ASP | 39.7# |
| 13ALA | 0.0 | 13ALA | 0.0 | 13ALA | 0.0 |
| 14ILEP¹ | 0.0 | 14ILE | 0.0 | 14ILE | 0.0 |
| I5ASN | 23.7 | 15ASN | 24.9 | 15ASN | 19.2 |
| 16TYR | 29.5 | 16TYR | 47.1# | 16TYR | 52.5# |
| 17TRP | 14.1 | 17TRP | 18.2 | 17TRP | 20.1 |
| 18GLY | 4.3 | 18GLY | 1.4 | 18GLY | 1.0 |
| 19PRO | 63.6# | 19PRO | 57.3# | 19PRO | 59.0# |
| 20LYS | 57.3# | 20LYS | 77.2# | 20LYS | 100# |
| 21ASN | 36.3# | 21ASN | 28.4 | 21ASN | 61.5# |
| 22ASNP¹ | 16.9 | 22ASN | 10.1 | 22ASN | 15.2 |
| 23ASNP¹ | 0.3 | 23ASN | 0.8 | 23ASN | 0.0 |
| 24GLYP¹ | 42.0# | 24GLY | 43.2# | 24GLY | 43.3# |
| 25ILEP¹ | 10.1 | 25ILE | 13.8 | 25ILE | 7.6 |
| 26GLNP¹ | 92.4# | 26GLN | 86.2# | 26GLN | 94.7# |
| 27GLYP¹ | 62.0# | 27GLY | 73.9# | 27GLY | 62.8# |
| 28GLY | 49.0# | 28GLY | 50.6# | 28TYR | 47.7# |
| 29ASP | 66.0# | 29ASP | 68.1# | 29ASN | 80.7# |
| 30PHEP¹ | 4.5 | 30PHE | 4.1 | 30PHE | 1.5 |
| 31GLY | 37.2# | 31GLY | 41.4# | 31ASN | 61.1# |
| 32TYR | 25.2 | 32TYR | 25.3 | 32TYR | 21.5 |
| 33PRO | 70.7# | 33PRO | 76.0# | 33PRO | 78.5# |
| 34ILE | 4.8 | 34ILE | 5.5 | 34ILE | 2.6 |
| 35SER | 42.2# | 35SER | 29.1 | 35SER | 27.0 |
| 36GLU | 54.2# | 36GLU | 47.2# | 36GLU | 50.2# |
| 37LYS | 81.0# | 37LYS | 79.5# | 37ARG | 87.5# |
| 38GLNP¹ | 12.8 | 38GLN | 14.5# | 38GLN | 9.0 |
| 39ILEP¹ | 7.8 | 39ILE | 7.9 | 39ILE | 5.1 |
| 40ASPP¹ | 52.4# | 40ASP | 55.3# | 40ASP | 49.8# |
| 41THRP¹ | 0.3 | 41THR | 0.0 | 41THR | 0.2 |
| 42SER | 53.1# | 42SER | 56.0# | 42SER | 53.0# |
| 43ILEP¹ | 13.1 | 43ILE | 23.5 | 43ILE | 25.1 |
| 44ILE | 8.3 | 44ILE | 12.0 | 44ILE | 8.1 |
| 45THR | 30.7 | 45THR | 37.8# | 45THR | 45.7# |
| 46PHE* | 20.0 | 46SER | 43.7# | 46SER | 40.5# |
| 47THR | 48.1# | 47THR | 45.2# | 47THR | 43.7# |
| 48HIS | 73.5# | 48HIS | 65.3# | 48HIS | 78.3# |
| 49PRO | 9.4 | 49PRO | 12.6 | 49SER | 9.0 |
| 50ARG | 58.7# | 50ARG | 53.7# | 50ARG | 61.5# |
| 51LEU | 13.7 | 51LEU | 8.1 | 51LEU | 3.0 |
| 52ILE* | 32.4 | 52ILE | 31.5 | 52MET | 43.7# |
| 53PRO | 22.2 | 53PRO | 26.5 | 53PRO | 22.8 |
| 54TYR* | 52.7# | 54HIS | 42.2# | 54HIS | 45.5# |
| 55ASP | 57.5# | 55ASP | 59.2# | 55ASP | 55.5# |
| 56LEU | 15.0 | 56LEU | 18.6 | 56LEU | 15.1 |
| 57THR | 62.0# | 57THR | 73.2# | 57THR | 80.0# |
| 58ILE | 67.6# | 58ILE | 60.9# | 58ILE | 68.0# |
| 59PRO | 26.6 | 59PRO | 21.9 | 59PRO | 20.7 |
| 60GLN | 28.8 | 60GLN | 20.7 | 60GLN | 21.3 |
| 61ASN | 71.9# | 61ASN | 73.6# | 61ASN | 74.6# |
| 62LEU | 13.4 | 62LEU | 11.7 | 62LEU | 10.0 |
| 63GLU | 62.2# | 63GLU | 66.0# | 63GLU | 66.4# |
| 64THR | 51.2# | 64THR | 51.1# | 64THR | 49.0# |
| 65ILE | 46.1# | 65ILE | 41.5# | 65ILE | 38.4# |
| 66PHE | 27.0 | 66PHE | 26.3 | 66PHE | 29.6 |
| 67THR | 52.5# | 67THR | 55.5# | 67THR | 62.2# |
| 68THR* | 31.6 | 68THR | 35.8# | 68THR | 35.3 |
| 69THR | 54.3# | 69THR | 51.1# | 69THR | 50.3# |
| 70GLN* | 31.0 | 70GLN | 36.5# | 70GLN | 34.3 |
| 71VAL | 53.4# | 71VAL | 53.2# | 71VAL | 53.1# |
| 72LEU | 7.9 | 72LEU | 11.8 | 72LEU | 8.1 |
| 73THR | 40.5# | 73THR | 44.0# | 73THR | 47.0# |
| 74ASN | 0.6 | 74ASN | 0.0 | 74ASN | 0.0 |
| 75ASN | 69.8# | 75ASN | 63.2# | 75ASN | 65.9# |
| 76THR | 36.7# | 76THR | 40.5# | 76THR | 44.0# |
| 77ASP | 80.2# | 77ASP | 63.0# | 77ASP | 62.1# |
| 78LEU | 62.6# | 78LEU | 62.5# | 78VAL | 63.8# |
| 79GLN | 74.4# | 79GLN | 54.7# | 79GLN | 43.5# |
| 80GLN | 33.3 | 80GLN | 32.1 | 80GLN | 40.1# |
| 81SER | 81.0# | 81SER | 68.6# | 81SER | 75.2# |
| 82GLN | 19.0 | 82GLN | 23.9 | 82GLN | 24.6 |
| 83THR | 62.7# | 83THR | 63.6# | 83THR | 63.7# |
| 84VAL | 1.8 | 84VAL | 0.9 | 84VAL | 0.0 |
| 85SER | 50.8# | 85SER | 55.7# | 85SER | 54.0# |
| 86PHE | 7.2 | 86PHE | 5.8 | 86PHE | 4.0 |
| 87ALA* | 58.5# | 87ALA | 61.9# | 87SER | 68.0# |
| 88LYS | 30.5 | 88LYS | 30.6 | 88LYS | 32.6 |
| 89LYS | 69.8# | 89LYS | 67.8# | 89LYS | 67.9# |
| 90THR | 19.9 | 90THR | 23.1 | 90THR | 16.7 |
| 91THR | 54.1# | 91THR | 55.1# | 91THR | 48.1# |
| 92THR | 1.8 | 92THR | 1.8 | 92THR | 0.0 |
| 93THRP²* | 40.3# | 93THR | 36.4# | 93THR | 29.4 |
| 94THR | 0.0 | 94THR | 0.6 | 94THR | 0.2 |
| 95SERP²* | 14.4 | 95SER | 15.7 | 95SER | 18.1 |
| 96THR | 5.5 | 96THR | 1.5 | 96THR | 0.0 |
| 97SERP² | 16.6 | 97SER | 18.5 | 97SER | 29.9 |
| 98THR | 8.2 | 98THR | 5.9 | 98THR | 1.9 |
| 99THR | 41.8# | 99THR | 46.4# | 99THR | 49.3# |
| 100ASN | 37.7# | 100ASN | 34.1 | 100ASP | 20.1 |
| 101GLY | 1.0 | 101GLY | 1.9 | 101GLY | 0.0 |
| 102TRP | 3.6 | 102TRP | 10.4 | 102TRP | 6.6 |
| 103THR | 8.1 | 103THR | 8.1 | 103THR | 3.9 |
| 104GLU | 9.7 | 104GLU | 21.9 | 104GLU | 14.8 |
| 105GLY* | 35.3 | 105GLY | 46.8# | 105GLY | 31.4 |
| 106GLY | 57.0# | 106GLY | 68.6# | 106GLY | 61.8# |
| 107LYS | 52.4# | 107LYS | 57.2# | 107ARG | 54.6# |
| 108ILE | 61.8# | 108ILE | 63.5# | 108ILE | 67.1# |
| 109SER | 43.5# | 109SER | 47.9# | 109SER | 47.7# |
| 110ASP | 83.1# | 110ASP | 83.5# | 110ASP | 65.7# |
| 111THR | 43.4# | 111THR | 41.3# | 111THR | 39.9# |
| 112LEU | 26.7 | 112LEU | 29.6 | 112LEU | 31.3 |
| 113GLU | 53.8# | 113GLU | 64.1# | 113GLU | 62.5# |
| 114GLU | 34.8 | 114GLU | 30.9 | 114GLU | 32.6 |
| 115LYS | 62.2# | 115LYS | 55.2# | 115ASN | 54.6# |
| 116VAL | 6.4 | 116VAL | 8.6 | 116VAL | 10.4 |
| 117SER* | 46.6# | 117SER | 48.9# | 117SER | 51.2# |
| 118VAL | 0.9 | 118VAL | 2.5 | 118VAL | 1.3 |
| 119SER* | 20.2 | 119SER | 23.4 | 119SER | 23.7 |
| 120ILE | 0.8 | 120ILE | 0.3 | 120ILE | 0.0 |
| 121PRO | 5.9 | 121PRO | 10.7 | 121PRO | 8.0 |
| 122PHE | 0.2 | 122PHE | 1.4 | 122PHE | 0.3 |
| 123ILE | 19.1 | 123ILE | 20.8 | 123ILE | 18.3 |
| 124GLY | 4.3 | 124GLY | 6.2 | 124GLY | 3.3 |
| 125GLU* | 59.7# | 125GLU | 56.2# | 125ALA | 57.8# |
| 126GLY | 50.0# | 126GLY | 52.5# | 126GLY | 49.6# |
| 127GLY | 47.2# | 127GLY | 56.7# | 127GLY | 38.6# |
| 128GLY* | 34.7 | 128GLY | 30.3 | 128ALA | 23.0 |
| 129LYS | 68.8# | 129LYS | 73.9# | 129LYS | 78.4# |
| 130ASN | 16.2 | 130ASN | 14.6 | 130ASN | 10.1 |
| 131SER | 78.2# | 131SER | 77.9# | 131SER | 80.3# |
| 132THR | 9.8 | 132THR | 10.3 | 132THR | 12.3 |
| 133THR* | 45.7# | 133THR | 42.0# | 133THR | 44.3# |
| 134ILE* | 1.1 | 134ILE | 0.8 | 134ILE | 0.0 |
| 135GLU* | 51.5# | 135GLU | 45.2# | 135GLU | 48.5# |
| 136ALA | 0.0 | 136ALA | 1.3 | 136ALA | 2.4 |
| 137ASN* | 18.1 | 137ASN | 15.5 | 137ASN | 15.6 |
| 138PHE* | 1.9 | 138PHE | 0.9 | 138VAL | 2.5 |
| 139ALA* | 2.8 | 139ALA | 6.3 | 139ALA | 4.1 |
| 140HIS | 2.3 | 140HIS | 2.1 | 140HIS | 0.0 |
| 141ASN | 5.3 | 141ASN | 6.5 | 141ASN | 2.8 |
| 142SER | 5.4 | 142SER | 4.4 | 142SER | 6.6 |
| 143SER | 7.7 | 143SER | 10.6 | 143SER | 7.0 |
| 144THR | 23.5 | 144THR | 17.3 | 144THR | 16.6 |
| 145THR* | 48.3# | 145THR | 52.7# | 145THR | 55.2# |
| 146THR | 50.2# | 146THR | 49.7# | 146THR | 53.6# |
| 147PHEP²* | 49.9# | 147PHE | 61.6# | 147SER | 51.7# |
| 148GLN* | 12.9 | 148GLN | 17.8 | 148GLN | 18.4 |
| 149GLNP²* | 59.5# | 149GLN | 65.1# | 149GLN | 69.1# |
| 150ALA* | 6.9 | 150ALA | 8.7 | 150ALA | 9.1 |
| 151SERP²* | 51.0# | 151SER | 51.7# | 151SER | 57.9# |
| 152THR | 9.9 | 152THR | 8.7 | 152THR | 12.3 |
| 153ASP* | 83.5# | 153ASP | 84.5# | 153GLU | 63.3# |
| 154ILE | 11.2 | 154ILE | 6.1 | 154ILE | 6.3 |
| 155GLU* | 49.5# | 155GLU | 63.9# | 155GLU | 49.7# |
| 156TRP | 1.7 | 156TRP | 3.8 | 156TRP | 1.8 |
| 157ASN* | 59.1# | 157ASN | 59.1# | 157ASN | 53.4# |
| 158ILE* | 13.1 | 158ILE | 5.9 | 158ILE | 0.8 |
| 159SER* | 60.2# | 159SER | 52.9# | 159SER | 52.2# |
| 160GLN | 29.2 | 160GLN | 19.3 | 160GLN | 9.3 |
| 161PRO | 54.0# | 161PRO | 63.6# | 161PRO | 62.6# |
| 162VAL | 0.6 | 162VAL | 4.0 | 162VAL | 2.4 |
| 163LEU | 53.8# | 163LEU | 56.6# | 163LEU | 64.5# |
| 164VAL | 0.0 | 164VAL | 0.0 | 164VAL | 0.0 |
| 165PRO | 22.8 | 165PRO | 22.1 | 165PRO | 26.9 |
| 166PRO | 30.7 | 166PRO | 36.1# | 166PRO | 39.7# |
| 167SER* | 31.0 | 167ARG | 32.8 | 167ARG | 36.7# |
| 168LYS | 18.2 | 168LYS | 18.5 | 168LYS | 19.9 |
| 169GLN | 17.4 | 169GLN | 15.1 | 169GLN | 10.7 |
| 170VAL | 0.0 | 170VAL | 0.0 | 170VAL | 0.0 |
| 171VAL | 13.2 | 171VAL | 13.8 | 171VAL | 12.2 |
| 172ALA | 0.0 | 172ALA | 0.0 | 172ALA | 0.0 |
| 173THR | 9.8 | 173THR | 9.2 | 173THR | 6.5 |
| 174LEU | 1.3 | 174LEU | 2.6 | 174LEU | 0.2 |
| 175VAL* | 17.2 | 175VAL | 17.8 | 175VAL | 13.4 |
| 176ILE | 0.0 | 176ILE | 0.0 | 176ILE | 0.0 |
| 177MET* | 7.0 | 177MET | 7.7 | 177MET | 17.3 |
| 178GLY | 1.6 | 178GLY | 0.5 | 178GLY | 0.0 |
| 179GLY | 15.9 | 179GLY | 22.2 | 179GLY | 16.5 |
| 180ASNP²* | 60.0# | 180ASN | 60.1# | 180ASP | 44.9# |
| 181PHE | 0.7 | 181PHE | 2.8 | 181PHE | 1.8 |
| 182THRP²* | 50.6# | 182THR | 44.3# | 182THR | 40.8# |
| 183ILE | 0.0 | 183ILE | 1.1 | 183VAL | 0.0 |
| 184PRO | 36.6# | 184PRO | 34.2 | 184PRO | 34.5 |
| 185MET | 4.4 | 185MET | 2.1 | 185MET | 1.8 |
| 186ASP | 52.4# | 186ASP | 23.5 | 186ASP | 20.3 |
| 187LEU* | 0.8 | 187LEU | 0.0 | 187LEU | 0.0 |
| 188MET | 25.9 | 188MET | 12.7 | 188ILE | 24.9 |
| 189THR | 1.4 | 189THR | 2.9 | 189THR | 0.5 |
| 190THR | 26.1 | 190THR | 26.2 | 190THR | 24.1 |
| 191ILE | 4.0 | 191ILE | 6.2 | 191ILE | 1.8 |
| 192ASP | 25.9 | 192ASP | 29.2 | 192ASP | 21.4 |
| 193SERP¹ | 7.4 | 193SER | 7.7 | 193SER | 2.7 |
| 194THRP¹ | 66.2# | 194THR | 60.2# | 194THR | 59.9# |
| 195GLUP¹ | 38.5# | 195GLU | 35.0 | 195GLN | 35.5# |
| 196HIS* | 37.7# | — | 100 | — | 100# |
| 197TYR* | 32.2 | — | 100# | — | 100# |
| 198SER* | 35.5# | — | 100# | — | 100# |
| 199HISP¹* | 64.3# | 196HIS | 55.5# | 196HIS | 51.7# |
| 200TYRP¹* | 85.3# | 197TYR | 56.2# | 197PHE | 45.8# |
| 201SERP¹* | 50.3# | 198SER | 68.3# | 198THR | 64.5# |
| 202GLYP¹ | 32.8 | 199GLY | 50.0# | 199GLY | 51.1# |
| 203TYRP¹ | 21.5 | 200TYR | 22.6 | 200TYR | 26.0 |
| 204PROP¹ | 1.4 | 201PRO | 1.0 | 201PRO | 1.0 |
| 205ILEP¹ | 1.1 | 202ILE | 0.3 | 202ILE | 0.0 |
| 206LEUP¹ | 1.8 | 203LEU | 2.6 | 203LEU | 0.0 |
| 207THRP¹ | 0.0 | 204THR | 0.0 | 204THR | 0.0 |
| 208TRPP¹* | 38.8# | 205TRP | 35.6# | 205TRP | 22.5 |
| 209ILEP¹ | 0.0 | 206ILE | 0.0 | 206ILE | 0.0 |
| 210SERP¹ | 22.5 | 207SER | 20.1 | 207GLU | 17.0 |
| 211SER | 3.1 | 208SER | 3.4 | 208ASN | 4.6 |
| 212PRO | 56.5# | 209PRO | 58.4# | 209PRO | 56.2# |
| 213ASP | 68.0# | 210ASP | 55.2# | 210GLU | 60.5# |
| 214ASN | 65.5# | 211ASN | 66.4# | 211HIS | 64.4# |
| 215SER | 67.2# | 212SER | 74.1# | 212ASN | 74.2# |
| 216TYRP¹ | 42.5# | 213TYR | 39.8# | 213VAL | 29.4 |
| 217SERP¹* | 43.2# | 214ASN | 46.3# | 214ARG | 57.1# |
| 218GLYP¹ | 1.2 | 215GLY | 4.1 | 215GLY | 6.1 |
| 219PROP¹* | 14.4 | 216PRO | 14.7 | 216ARG | 33.7 |
| 220PHEP¹ | 0.0 | 217PHE | 0.0 | 217PHE | 0.0 |
| 221METP¹ | 15.2 | 218MET | 16.1 | 218LEU | 8.4 |
| 222SERP¹ | 3.3 | 219SER | 3.3 | 210SER | 0.0 |
| 223TRPP¹* | 35.6# | 220TRP | 34.3 | 220TRP | 42.5# |
| 224TYRP¹ | 13.5 | 221TYR | 15.9 | 221PHE | 11.3 |
| 225PHEP¹ | 0.9 | 222PHE | 1.4 | 222PHE | 0.0 |
| 226ALA | 15.9 | 223ALA | 13.1 | 223ALA | 7.8 |
| 227ASN | 40.7# | 224ASN | 41.9# | 224ASN | 43.2# |
| 228TRP | 9.0 | 225TRP | 8.9 | 225TRP | 7.3 |
| 229PRO | 56.3# | 226PRO | 61.5# | 226PRO | 65.5# |
| 230ASN | 67.6# | 227ASN | 67.3# | 227ASN | 67.3# |
| 231LEU | 21.1 | 228LEU | 16.1 | 228LEU | 16.1 |
| 232PRO | 23.7 | 229PRO | 23.0 | 229PRO | 23.6 |
| 233SER | 97.0# | 230SER | 95.8# | 230SER | 88.1# |
| 234GLY | 23.0 | 231GLY | 19.5 | 231GLU | 13.0 |
| 235PHE* | 8.3 | 232PHE | 9.0 | 232PHE | 6.1 |
| 236GLY | 26.1 | 233GLY | 18.3 | 233GLY | 28.5 |
| 237PRO | 72.6# | 234PRO | 70.8# | 234SER | 81.9# |
| 238LEU | 27.7 | 235LEU | 28.1 | 235LEU | 25.9 |
| 239ASN* | 33.2 | 236ASN | 26.5 | 236ASN | 42.2# |
| 240SER | 100# | 237SER | 100# | 237SER | 100# |
| 241ASP* | 62.2# | 238ASP | 61.7# | 238ASP | 55.2# |
| 242ASN | 15.1 | 239ASN | 20.8 | 239ASN | 21.7 |
| 243THR* | 3.3 | 240THR | 2.4 | 240THR | 2.6 |
| 244VAL* | 1.8 | 241VAL | 3.1 | 241ILE | 0.0 |
| 245THR* | 19.0 | 242THR | 23.8 | 242THR | 30.1 |
| 246TYR* | 8.6 | 243TYR | 4.8 | 243TYR | 0.4 |
| 247THR* | 36.8# | 244THR | 40.8# | 244LYS | 58.8# |
| 248GLY | 2.5 | 245GLY | 1.4 | 245GLY | 0.0 |
| 249SER* | 20.7 | 246SER | 23.9 | 246SER | 27.0 |
| 250VAL* | 6.0 | 247VAL | 1.4 | 247VAL | 0.0 |
| 251VALP¹* | 32.6 | 248VAL | 30.0 | 248VAL | 29.7 |
| 252SER* | 0.0 | 249SER | 0.0 | 249SER | 0.0 |
| 253GLNP¹ | 40.2# | 250GLN | 37.6# | 250ARG | 51.3# |
| 254VAL | 1.2 | 251VAL | 1.5 | 251ILE | 2.7 |
| 255SERP¹ | 35.3 | 252SER | 37.3# | 252SER | 43.7# |
| 256ALA | 6.1 | 253ALA | 2.4 | 253ALA | 2.1 |
| 257GLY | 4.1 | 254GLY | 6.1 | 254GLY | 1.2 |
| 258VAL | 0.3 | 255VAL | 0.0 | 255VAL | 0.0 |
| 259TYR | 2.2 | 256TYR | 1.1 | 256TYR | 1.0 |
| 260ALA | 0.7 | 257ALA | 0.7 | 257ALA | 0.0 |
| 261THR | 9.2 | 258THR | 9.3 | 258THR | 5.0 |
| 262VAL | 3.6 | 259VAL | 1.5 | 259VAL | 0.2 |
| 263ARG | 26.6 | 260ARG | 29.8 | 260ARG | 26.9 |
| 264PHE | 0.5 | 261PHE | 3.8 | 261PHE | 1.5 |
| 265ASP | 6.9 | 262ASP | 7.2 | 262ASP | 10.8 |
| 266GLN | 5.6 | 263GLN | 5.8 | 263GLN | 2.6 |
| 267TYR | 16.1 | 264TYR | 14.5 | 264TYR | 12.4 |
| 268ASP | 29.8 | 265ASP | 31.4 | 265ALA | 19.3 |
| 269ILE | 25.4 | 266ILE | 18.2 | 266ILE | 14.5 |
| 270HIS | 85.5# | 267HIS | 72.2# | 267ASN | 92.0# |
| 271ASN | 43.4# | 268ASN | 46.9# | 268ASN | 64.0# |
| 272LEU | 40.3# | 269LEU | 43.1# | 269LEU | 39.8# |
| 273ARG* | 86.3# | 270ARG | 63.1# | 270ARG | 84.4# |
| 274THR* | 52.0# | 271THR | 66.1# | 271THR | 76.8# |
| 275ILE* | 41.0# | 272ILE | 37.9# | 272ILE | 32.4 |
| 276GLU | 47.9# | 273GLU | 50.1# | 273GLU | 53.0# |
| 277LYS | 49.8# | 274LYS | 47.2# | 274LYS | 70.2# |
| 278THR | 46.3# | 275THR | 51.2# | 275THR | 53.7# |
| 279TRP | 25.2 | 276TRP | 25.0 | 276TRP | 33.4 |
| 280TYR | 35.5 | 277TYR | 30.7 | 277TYR | 21.3 |
| 281ALA | 6.6 | 278ALA | 7.9 | 278ALA | 4.4 |
| 282ARG* | 77.6# | 279ARG | 80.6# | 279ARG | 86.1# |
| 283HIS | 45.2# | 280HIS | 36.6# | 280HIS | 35.8# |
| 284ALA | 0.8 | 281ALA | 0.8 | 281GLY | 0.6 |
| 285THR | 14.7 | 282THR | 8.6 | 282THR | 2.0 |
| 286LEU | 3.6 | 283LEU | 5.9 | 283LEU | 2.3 |
| 287HIS* | 8.4 | 284HIS | 16.5 | 284HIS | 11.9 |
| 288ASN | 40.4# | 285ASN | 43.9# | 285ASN | 38.7# |
| 289GLY | 61.0# | 286GLY | 53.5# | 286GLY | 61.1# |
| 290LYS | 61.7# | 287LYS | 61.6# | 287LYS | 73.8# |
| 291LYS | 68.4# | 288LYS | 66.2# | 288LYS | 51.9# |
| 292ILE | 19.2 | 289ILE | 19.5 | 289ILE | 21.0 |
| 293SER* | 40.3# | 290SER | 47.9# | 290SER | 45.7# |
| 294ILE | 3.4 | 291ILE | 4.8 | 291ILE | 5.1 |
| 295ASN* | 29.3 | 292ASN | 21.9 | 292ASN | 18.0 |
| 296ASN | 38.2# | 293ASN | 40.4# | 293ASN | 37.4# |
| 297VAL | 1.3 | 294VAL | 1.4 | 294VAL | 0.7 |
| 298THR | 10.1 | 295THR | 9.5# | 295THR | 4.3 |
| 299GLU* | 77.1# | 296GLU | 72.7# | 296GLU | 68.8# |
| 300MET* | 48.6# | 297MET | 46.4# | 297MET | 42.8# |
| 301ALA | 65.3# | 298ALA | 54.1# | 298ALA | 60.4# |
| 302PRO | 66.0# | 299PRO | 73.0# | 299PRO | 77.8# |
| 303THR* | 83.7# | 300THR | 85.8# | 300THR | 94.1# |
| 304SER | 77.4# | 301SER | 76.1# | 301SER | 84.9# |
| 305PRO* | 81.1# | 302PRO | 65.7# | 302PRO | 83.4# |
| 306ILE* | 78.1# | 303ILE | 81.6# | 303ILE | 91.3# |
| 307LYS | 81.9# | 304LYS | 99.3# | 304GLU | 100# |
| 308THR* | 89.4# | 305THR | 100# | 305ARG | 100# |
| 309ASN | 100# | 306ASN | 100# | 306ASN | 100# |

P1 designates an amino acid in surface patch [1] of FIG. 2.
P2 designates an amino acid in surface patch [2] of FIG. 2.
*designates one of the 72 principally relevant amino acids described herein (see FIG. 2).
Shown are residues of TIC807_Ll1M, TIC807_M2, and TIC853 aligned by Clustal W.
Numbers marked with # represent % SA of at least about 36%.

### Receptor Binding

A surface patch ([1] of FIG. 2) of residues having % SA values greater than 36% or within about 3 residues of a residue having % SA greater than 36% in a radius of about 9.2-12.2 Angstroms from the Cb atom of S95 of SEQ ID NO:2 (TIC807) was identified as a region comprising residues of a TIC807 protein that can be substituted to provide for eHTP's that exhibit enhanced Lygus inhibitory spectrum and/or improved Lygus inhibitory activity, This surface patch region may be associated with target insect receptor binding activity; and, includes residues T93, S95, S97, F147, Q149, S151, N180, T182, V251, Q253, and S255 of SEQ ID NO:2 (TIC807). eHTP's can include, but are not limited to, one or more substitutions of surface patch 1 amino acid residues such as S95A, F147A, Q149E, and/or, V251A.

The combined engineering-testing-selecting approaches described herein identified residues located in surface patch 1 that can provide for eHTP's when substituted or otherwise modified. These residues may be important for productive binding of eHTP's to receptors in Lygus insect gut to provide for enhanced Lygus inhibitory spectrum and/or improved Lygus inhibitory activity when compared to TIC807. Modifications of the surface patch 1 amino acid residues that can provide for eHTP's include substitutions that provide aromatic groups and/or hydrogen-bonding groups which favoring binding to sugar groups found on glycosylated receptors of insects.

### Membrane Binding

Certain amino acid residues located in beta-sheet regions of the protein were identified from the atomic structure of TIC807 and were substituted with aromatic residues. More specifically, amino acids L78, 1123, H270, 8273, I275 of the folded TIC807 beta sheet regions were substituted with Phenyalanine, Tyrosine, or Tryptophan. Aromatic amino acid substitutions of 8273 and 1275 were amongst those residues that provided for an enhanced Lygus inhibitory spectrum and/or improved Lygus inhibitory activity (See Table 4, data for SEQ ID NOs: 32, 34, 68, 92, and 122). Amino acid side chains of residues in these positions may be likely to interact with the membrane of target insects.

### Proteolytic Activation Sites

Glycine residues generally thought to be involved in proteolysis were substituted with Serines to alter proteolytic cleavage dynamics. The presence of a glycine residue in a loop region can impart more flexibility and therefore susceptibility to proteolysis, which can either increase insect inhibitory activity or decrease insect inhibitory activity. Residues in structurally identified loop regions were substituted with a glycine residue, and no improvements were observed. Positions in loops that were already glycines, (e.g. G18, G24, G27) were substituted with a serine, a small residue in an attempt to reduce proteolytic susceptibility, and no improvements were observed.

### Combined Structure Design Approaches

The atomic structure of TIC807 (SEQ ID NO:2) was used to identify loop regions for library mutagenesis followed by testing of the engineered variants. A loop at amino acid positions 211-216 of SEQ ID NO:2 (TIC807) was library-mutagenized and tested. Consecutive loops in close proximity at amino acid positions 75-83, 161-167, and 267-276 of SEQ ID NO:2 (TIC807) was library-mutagenized and tested.

Analysis of the atomic structure of TIC807 suggests that a structural loop resides at residues 113-138 of SEQ ID NO:2, and variants were engineered to stabilize and destabilize the loop.

In another region spanning two beta-strands connected by a short loop, the two beta-strands exhibited an alternating pattern of hydrophobic and hydrophilic amino acid residues at positions 116 to 121 and at positions 133 to 138 relative to SEQ ID NO:2, characteristic of pore-forming loops. An expression library was engineered to modify both beta-strand segments replacing residues V116, V118, and 1120 with respective combinations 116V/Y/L/H/F/D, 118V/Y/L/H/F/D, and 120I/D/F/H/L/NN/Y for a total of 288 possible variants in the library. This procedure was repeated for: residues 5117, 5119, and P121 with respective combinations 1175/A/D/E/G/K/N/R/T, 1195/A/D/E/G/K/N/R/T, and 121P/S/T for 243 potential variants; residues 1133, A135, and F137 with respective combinations 133I/D/F/NN/Y, 135A/D/F/H/L/V/Y, and 137F/D/H/L/V/Y for 252 possible variants; and residues T134, E136, and N138 with respective combinations 134T/A/D/E/G/K/N/R/S, 136E/A/D/G/K/N/R/S/T, and 138N/A/D/G/S/T for 486 possible variants. An enhanced Lygus inhibitory spectrum and/or improved Lygus inhibitory activity was associated with certain of these substitutions as shown in Table 4.

### Structure-Function Relationship

Altogether, more than 2000 clones (including mixed library clones) expressing variants of TIC807 were tested for enhanced Lygus inhibitory spectrum and/or improved Lygus inhibitory activity against Lygus spp. compared to TIC807. Semi-random modifications, directed modifications, and predictive structure-function modifications, including structure modeling, receptor binding potential, metal binding potential, oligomerization potential, uniformity of surface charge distribution, pore formation potential, ion channel function, and identification of surface exposed patches to with an objective of identifying eHTP's with an enhanced Lygus inhibitory spectrum and/or improved Lygus inhibitory activity compared to TIC807. These clones were expressed for bioassay testing.

### Example 3: Protein Expression and Purification of TIC807, Including Variants and Fragments

Control protein TIC807 is a protein of 309 amino acids in length that can be expressed in crystalline form in Bacillus thuringiensis (Bt) or aggregate form in E. coli. Test variants thereof were recombinantly expressed in Bt. An expression characteristic of TIC807 and variants of TIC807 is the predominant crystalline and aggregate forms extracted from Bt and E. coli cells, respectively. To test for Lygus bioactivity, test and control samples were made suitable for Lygus bioassay by solubilizing samples in 25 mM Sodium Carbonate buffer and removing unsolubilized materials by centrifugation. The amount of protein in test and control samples were measured using total protein methods, e.g.s, a Bradford assay, an ELISA method, or similar. Gel electrophoresis was used to determine the purity and stock concentration of the solubilized recombinant protein. C-terminal HIS-tagged TIC807 protein was engineered to facilitate detection, purification, and quantification of large amounts of TIC807 control protein. C-terminal HIS-tagged TIC807 and un-tagged TIC807 test samples were separately assayed and confirmed to have equivalent activity against Lygus (see Examples 4, 5, and 6).

Site-directed amino acid substitutions were made to TIC807 M13 (SEQ ID NO:34) to elevate expression of a soluble form. Inventors postulate that more readily soluble variants of the proteins of the present invention can facilitate expression and purification, e.g., expressed in E. coli host cells; and can increase insect inhibitory efficacy when expressed in plant host cells. Recombinant DNA constructs encoding TIC807 M13 (SEQ ID NO:34) were engineered three different ways to reflect three different variants: Relative to TIC807 M13, the modifications were for Variant #1: I58K and P59K, for Variant #2: S198K and G199K, and for Variant #3: S246R, V248E, and Q250R. Relative to TIC807 (SEQ ID NO:2), the modifications can be alternatively described as follows for Variant #1: I58K and P59K, for Variant #2: S201K and G202K, and for Variant #3: S249R, V251E, and Q253R; this positional difference is congruent due to a contiguous triple deletion of SEQ ID NO:2 (TIC807) in residue range 196-201 that is reflected in TIC807 M13 (SEQ ID NO:34). The four engineered recombinant DNA constructs were each cloned and expressed in E. coli. The soluble fraction from the four E. coli preparations were evaluated by coomassie-stained SDS-PAGE, which showed that TIC807 M13 (SEQ ID NO:34) was not detectable in the soluble fraction; but, in contrast, Variant #s 1, 2, and 3 were soluble. Similar amino acid substitutions either singly or in combination are made to proteins of the present invention to elevate their solubility in non-Bt or plant host cells. Recombinant DNA constructs were engineered to encode for and express TIC807 M13 variant #3 (renamed TIC807 M14; nucleotide SEQ ID NO:203 and amino acid SEQ ID NO:204). Prepared E. coli lysate was clarified, and the recombinant protein purified and enriched-for on a series of columns, including ion-exchange and gel filtration methods. Pooled protein fractions were quantified and determined to be active against Lygus insects (See Example 4, Table 4B).

Proteins of the present invention, including but not limited to proteins having the amino acid sequence as set forth as SEQ ID NO:28, SEQ ID NO:30, SEQ ID NO:32, or SEQ ID NO:36, are engineered to elevate expression of a soluble form when expressed in a host cell, e.g., expressed in Bt, E. coli, or in a plant cell or in a compartment of a plant cell. Engineering includes substituting a lysine amino acid residue at one or more of the following positions 58, 59, 198, 199, 201, or 202; or, a Glutamic acid at one or more of the following positions 198, 248, or 301; or, an Arginine at one or more of the following positions 246, 250, or 253.

The C-terminal region protrudes away from the monomeric core of the protein (See FIG. 2). A recombinant DNA construct was engineered to encode for and express a protein having the amino acid sequence of SEQ ID NO:202, which is a protein fragment (amino acids 1 to 301) of TIC807 M8 (SEQ ID NO:16); and, the expressed protein was purified, quantified, and determined active against Lygus insects (See Example 4, Table 4B). Recombinant DNA constructs were designed to encode for and express TIC807 fragments exhibiting varying truncations off of the C-terminus end of the proteins of the present invention at the respective TIC807 positions A281, G289, 5293, A301, and 5304. Protein fragments are engineered to encode for and express proteins having the amino acid sequences set forth as SEQ ID NO:28, SEQ ID NO:30, SEQ ID NO:32, SEQ ID NO:34, and SEQ ID NO:36; and, the expressed protein fragments are used as test samples against Lygus insects.

### Example 4: Hemipteran Activity of Engineered Proteins

This example illustrates eHTP's to have improved insecticidal activity or enhanced insecticidal specificity against Hemipteran insects when provided in the diet of Hemipteran insects, including but not limited to members of the Heteroptera miridae, including the genus Lygus, e.g., Lygus hesperus and Lygus lineolaris, and the family Cicadellidae, including the genus Amrasca, e.g. Amrasca devastans, and Empoasca, e.g. Empoasca fabae. This example with Table 4B illustrates the feeding assay used to determine the enhanced Lygus inhibitory spectrum and/or improved Lygus inhibitory activity of a Bt expressed recombinant proteins of the present invention against both Lygus hesperus and Lygus lineolaris. Proteins expressed in recombinant bacterium host cells were solubilized in carbonate buffer and analyzed by SDS polyacrylamide gel electrophoresis (SDS-PAGE); and, protein concentrations determined by densitometry using bovine serum albumin (BSA) as a standard. Protein stock (2×) prepared this way were mixed with diet for feeding assays.

Feeding assays with the Hemipteran species Lygus hesperus and Lygus lineolaris were based on a 96 well micro-titer plate format with Lygus diet encapsulated between stretched Parafilm® and Mylar sheets. Artificial diet was obtained from Bio-Sery® (Bio-Sery® Diet F9644B, Frenchtown, NJ). Autoclaved, boiling water (518 mL) was combined with 156.3 grams of Bio-Sery® diet F9644B in a surface-sterilized blender. The contents of four surface-sterilized chicken eggs were added and the mixture blended until smooth, then adjusted to one liter total volume and allowed to cool to room temperature, this being the 2× diet. Test samples were prepared by mixing in a 1:1 ratio of 2× diet and 2× sample. A sheet of Parafilm® (Pechiney Plastic Packing, Chicago, IL) was placed over a vacuum manifold designed for 96-well format (Analytical Research Systems, Gainesville, FL) and a vacuum of approximately −20 millimeters mercury was applied, sufficient to cause extrusion of the Parafilm® into the wells. Twenty to forty microliters of test sample were added to the Parafilm® extrusions. A sheet of Mylar film (Clear Lam Packaging, Inc., Elk Grove Village, IL) was placed over the sample filled Parafilm® extrusions and sealed with a tacking iron (Bienfang Sealector II, Hunt Corporation, Philadelphia, PA), thus forming diet filled Parafilm® sachets. These Parafilm® sachets were positioned over a flat-bottom 96-well plate containing Lygus eggs suspended in a dilute agarose solution. Upon hatching, Lygus nymphs feed on the diet by piercing the diet filled Parafilm® sachets. Alternatively, newly hatched Lygus nymphs instead of eggs were manually infested into each well. Stunting and mortality scores were determined on day 5 and compared to controls. Data were analyzed using JMP4 statistical software. For each protein at a test concentration, three populations of eight nymphs were subjected to this bioassay, and mortality scores reported in Table 4B.

For LC50 determinations listed in Table 1 and Table 4B, proteins were presented to newly hatched Lygus nymphs at 8-10 concentrations and the nymphs allowed to feed for 5 days before scoring for mortality over the dose range. For each concentration, three populations of eight nymphs were subjected to this bioassay, and all LC50 determinations in Table 1 and Table 4B were repeated at least once.

For LC50 estimations, proteins were presented to newly hatched Lygus lineolaris nymphs at 4 concentrations and the nymphs allowed to feed for 5 days before scoring for mortality over the dose range. Lygus lineolaris LC50 estimations were performed on TIC807 and TIC807 M2 because significantly large amounts of these proteins in excess of 1000 [μg/mL have not been possible to provide in Lygus diet in order to complete the high range of toxicity dose response to Lygus lineolaris; and therefore, an LC50 value was not determined for TIC807 or TIC807 M2. Instead, a 4-dose LC50 estimation in the low range was performed, and reported in Table 1 and Table 4B. The estimated Lygus lineolaris LC50 for TIC807 M14 is 4.4 μg/mL. For each concentration, three populations of eight nymphs were subjected to this bioassay.

This example with Tables 4A and 4B illustrate the feeding assay used to determine the enhanced inhibitory spectrum and/or improved inhibitory activity of a Bt expressed recombinant protein disclosed herein against Amrasca devastans. TIC807 variants with improved insecticidal activity or enhanced insecticidal specificity against Lygus hesperus and Lygus lineolaris exhibit improved insecticidal activity against Amrasca devastans.

TIC807, and TIC807-M13 were dissolved in 25 mM sodium carbonate buffer, pH 10. Amrasca devastans eggs were collected on Okra leaf and incubated in a petriplate containing 2% agar. Upon hatching the neonates were used for biossays using the diluted (1:5) Lygus diet. The proteins and diet were mixed at equal proportion (bringing final concentration of protein to 500 μg/mL) and dispensed into test arena. Untreated control was prepared by mixing the buffer with the diet. Individual neonates were infested into the test arena, the assays were incubated at 25° C., 60% RH. Twenty neonate nymphs were tested for each concentration, protein and in 2 replicates. A control was maintained with 25 mM Sodium Carbonate buffer, pH 10, in 1:5 diluted Lygus diet. Mortality of the insects was determined on the fifth day. Mortality values were calculated by the following formula: (% mortality in treatment−% mortality in control)/(100−% mortality in control)×100. Table 4A tabulates Amrasca activity for TIC807 and TIC807_M13 at 5 different concentrations.

TABLE 4A
TIC807 and TIC807_M13 Percent Mortality Directed to Amrasca species

| | | Mortality (%) | | | | |
|---|---|---|---|---|---|---|
| SEQ ID NO: | Protein Name | 500 μg/mL | 166.66 μg/mL | 55.55 μg/mL | 18.51 μg/mL | 6.17 μg/mL |
| 2 | TIC807 | 100% | 55.88% | 17.64% | 0 | 0 |
| 34 | TIC807_M13 | 100% | 88.23% | 73.52% | 44.11% | 26.47% |

LC50 values were determined for TIC807 and TIC807 M13 in a separate test. SEQ ID NO: 2 (TIC807) exhibited a LC50 value of 116.79 μg/mL and LC90 of 437.27 μg/mL. SEQ ID NO: 34 (TIC807 M13) exhibited a LC50 value of 7.59 μg/mL and LC90 value of 239.8 μg/mL.

A feeding assay as described for Amrasca devastans is used to test eHTP's for improved insecticidal activity and/or enhanced insecticidal specificity against Empoasca fabae. TIC807 variants with improved insecticidal activity or enhanced insecticidal specificity against Lygus hesperus and Lygus lineolaris exhibit improved insecticidal activity against Empoasca fabae.

The LC50 values of Cry51Aa1 (SEQ ID NO:182), for TIC807 (SEQ ID NO:2), TIC807_M2 (SEQ ID NO:8), TIC807 M10 (SEQ ID NO:30) and TIC807-M13 (SEQ ID NO:34) against Lygus hesperus and Lygus lineolaris were determined in one test. TIC807_M2, TIC807_M10 and TIC807_M12 exhibit improved LC50 values compared to Cry51Aa1.

It should be apparent to those skilled in the art that variations to this procedure can exist that should not affect results.

TABLE 4B
Iterative engineering-testing-selecting of eHTP's against Lygus spp. resulted in 267 proteins with enhanced
Lygus inhibitory spectrum and/or improved Lygus inhibitory activity against Lygus spp. compared to TIC807.

| | | | | | Lygus hesperus | | | Lygus lineolaris | | |
|---|---|---|---|---|---|---|---|---|---|---|
| | | Amino acid differences | | | | | % Mor- | | | % Mor- |
| | | compared to TIC807 parent protein | | | | | tality | | | tality |
| | | Amino acid | | | | fold | at | | fold | at |
| | | difference(s) | Amino acid | Amino acid | | in- | about | | in- | about |
| SEQ | | relative to | difference(s) | difference(s) | LC50 | creased | 1-3 | LC50 | creased | 100 |
| ID | | SEQ ID NO: 2 | in the | in the | value | toxicity | μg/mL | value | toxicity | μg/mL |
| NO: | Protein Name | (TIC807) | first patch | second patch | (μg/mL) | (LC50) | protein* | (μg/mL) | (LC50) | protein* |
| 2 | TIC807 | Parent | Parent | Parent | 73 | 1 | 0 | >223 | 1 | 0 |
| 6 | TIC807_M1 | F147A | F147A | None | 23 | 3 | ND | 100 | at | ND |
| | | | | | | | | | least | |
| | | | | | | | | | 2 | |
| 8 | TIC807_M2 | F46S, Y54H, S167R, | none | S217N, then a | 6.0 | 12 | 39 | >223 | — | 14 |
| | | S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 10 | TIC807_M3 | F46S, Y54H, T93A, | T93A | S217N, then a | 2.9 | 25 | 43 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 12 | TIC807_M4 | F46S, Y54H, S95A, | S95A | S217N, then a | 2.4 | 30 | 20 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 14 | TIC807_M5 | F46S, Y54H, F147A, | F147A | S217N, then a | 1.1 | 66 | 34 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 16 | TIC807_M8 | F46S, Y54H, S95A, | S95A, F147A | S217N, then a | 0.8 | 91 | ND | 223 | at | ND |
| | | F147A, S167R, | | contiguous triple | | | | | least | |
| | | S217N, then a | | deletion in residue | | | | | 1 | |
| | | contiguous triple | | range 196-201 | | | | | | |
| | | deletion in residue | | | | | | | | |
| | | range 196-201 | | | | | | | | |
| 18 | TIC807_M6 | F46S, Y54H, T93A, | T93A, F147A | S217N, then a | 1.5 | 50 | ND | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 20 | TIC807_M7 | F46S, Y54H, Q149E, | Q149E | S217N, then a | 1.4 | 52 | 62 | ND | — | 49 |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 28 | TIC807_M9 | F46S, Y54H, S95A, | S95A, F147A | P219R, then a | 9.9 | 7 | ND | 8.3 | at | ND |
| | | F147A, S167R, | | contiguous triple | | | | | least | |
| | | P219R, then a | | deletion in residue | | | | | 27 | |
| | | contiguous triple | | range 196-201 | | | | | | |
| | | deletion in residue | | | | | | | | |
| | | range 196-201 | | | | | | | | |
| 30 | TIC807_M10 | F46S, Y54H, S95A, | S95A, F147A, | P219R, then a | 0.6 | 122 | ND | 4.8 | at | ND |
| | | F147A, S167R, P219R, | V251A | contiguous triple | | | | | least | |
| | | V251A, then a | | deletion in residue | | | | | 46 | |
| | | contiguous triple | | range 196-201 | | | | | | |
| | | deletion in residue | | | | | | | | |
| | | range 196-201 | | | | | | | | |
| 32 | TIC807_M11 | F46S, Y54H, S95A, | S95A, F147A | P219R, then a | 1.4 | 54 | ND | 5.9 | at | ND |
| | | F147A, S167R, P219R, | | contiguous triple | | | | | least | |
| | | R273W, then a | | deletion in residue | | | | | 38 | |
| | | contiguous triple | | range 196-201 | | | | | | |
| | | deletion in residue | | | | | | | | |
| | | range 196-201 | | | | | | | | |
| 34 | TIC807_M13 | F46S, Y54H, S95A, | S95A, F147A, | P219R, then a | 0.3 | 243 | ND | 0.85 | at | ND |
| | | F147S, Q149E, S167R, | Q149E | contiguous triple | | | | | least | |
| | | P219R, R273W, then a | | deletion in residue | | | | | 262 | |
| | | contiguous triple | | range 196-201 | | | | | | |
| | | deletion in residue | | | | | | | | |
| | | range 196-201 | | | | | | | | |
| 36 | TIC807_M12 | F46S, Y54H, S95A, | S95A, F147A, | P219R, then a | 0.4 | 182 | ND | 1.2 | at | ND |
| | | F147A, S167R, P219R, | V251A | contiguous triple | | | | | least | |
| | | N239A, V251A, then a | | deletion in residue | | | | | 186 | |
| | | contiguous triple | | range 196-201 | | | | | | |
| | | deletion in residue | | | | | | | | |
| | | range 196-201 | | | | | | | | |
| 37 | TIC807_37 | TIC807_HYS_deletion | none | a contiguous triple | 22.4 | 3 | ND | ND | — | ND |
| | | | | deletion | | | | | | |
| | | | | in HYSHYS | | | | | | |
| | | | | residues (positions | | | | | | |
| | | | | 196-201) | | | | | | |
| 38 | TIC807_38 | F46S, Y54H, F138V, | none | S217N, then a | ND | — | 41 | ND | — | 44 |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 39 | TIC807_39 | F46S, Y54H, S167R, | none | S217N, then a | ND | — | 57 | ND | — | 31 |
| | | S217N, H287F, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 40 | TIC807_40 | F46S, I52M, Y54H, | none | S217N, then a | ND | — | 52 | ND | — | 36 |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 41 | TIC807_41 | N12D, F46S, Y54H, | none | S217N, then a | ND | — | 52 | ND | — | 31 |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 42 | TIC807_42 | F46S, Y54H, S167R, | N180D | S217N, then a | ND | — | 63 | ND | — | 50 |
| | | N180D, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 43 | TIC807_43 | F46S, Y54H, | none | a contiguous triple | ND | — | 46 | ND | — | 87 |
| | | S167R, then a | | deletion in | | | | | | |
| | | contiguous triple | | HYSHYS | | | | | | |
| | | deletion in residue | | residues (positions | | | | | | |
| | | range 196-201 | | 196-201) | | | | | | |
| 44 | TIC807_44 | F46S, Y54H, S167R, | none | S217N, P219R, then | ND | — | 30 | ND | — | 94 |
| | | S217N, P219R, then a | | a contiguous triple | | | | | | |
| | | contiguous triple | | deletion in residue | | | | | | |
| | | deletion in residue | | range 196-201 | | | | | | |
| | | range 196-201 | | | | | | | | |
| 45 | TIC807_45 | F46S, Y54H, S159T, | none | S217N, then a | ND | — | 67 | ND | — | 21 |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 46 | TIC807_46 | F46S, Y54H, S167R, | none | S217N, then a | ND | — | 52 | ND | — | 31 |
| | | S217N, T247K, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 47 | TIC807_47 | F46S, Y54H, S167R, | none | S217N, then a | ND | — | 59 | ND | — | 31 |
| | | S217N, V244I, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 48 | TIC807_48 | F46S, Y54H, S167R, | none | S217N, then a | ND | — | 58 | ND | — | 34 |
| | | S217N, V244I, | | contiguous triple | | | | | | |
| | | T247K, then a | | deletion in residue | | | | | | |
| | | contiguous triple | | range 196-201 | | | | | | |
| | | deletion in residue | | | | | | | | |
| | | range196-201 | | | | | | | | |
| 49 | TIC807_49 | F46S, Y54H, S167R, | none | S217N, W223Y, | ND | — | 17 | ND | — | 13 |
| | | S217N, W223Y, then a | | then a contiguous | | | | | | |
| | | contiguous triple | | triple deletion in | | | | | | |
| | | deletion in residue | | residue | | | | | | |
| | | range 196-201 | | range 196-201 | | | | | | |
| 50 | TIC807_50 | F46S, Y54H, S167R, | none | S217N, then a | ND | — | 48 | ND | — | 19 |
| | | S217N, Y246F, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 51 | TIC807_51 | F46S, Y54H, F147A, | F147A | S217N, then a | ND | — | 77 | ND | — | ND |
| | | G128A, S167R, S217N, | | contiguous triple | | | | | | |
| | | then a contiguous triple | | deletion in residue | | | | | | |
| | | deletion in residue | | range 196-201 | | | | | | |
| | | range 196-201 | | | | | | | | |
| 52 | TIC807_52 | F46S, Y54H, F147A, | F147A | S217N, then a | ND | — | 73 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 53 | TIC807_53 | F46S, Y54H, F147A, | F147A | S217N, then a | ND | — | 73 | ND | — | ND |
| | | S167R, S217N, | | contiguous triple | | | | | | |
| | | S293A, then a | | deletion in residue | | | | | | |
| | | contiguous triple | | range 196-201 | | | | | | |
| | | deletion in residue | | | | | | | | |
| | | range 196-201 | | | | | | | | |
| 54 | TIC807_54 | F46S, Y54H, F147A, | F147A | S217N, then a | ND | — | 67 | ND | — | ND |
| | | S167R, S217N, | | contiguous triple | | | | | | |
| | | H287A, then a | | deletion in residue | | | | | | |
| | | contiguous triple | | range 196-201 | | | | | | |
| | | deletion in residue | | | | | | | | |
| | | range 196-201 | | | | | | | | |
| 55 | TIC807_55 | F46S, Y54H, F147A, | F147A | S217N, then a | ND | — | 65 | ND | — | ND |
| | | S167R, S217N, | | contiguous triple | | | | | | |
| | | T274A, then a | | deletion in residue | | | | | | |
| | | contiguous triple | | range 196-201 | | | | | | |
| | | deletion in residue | | | | | | | | |
| | | range 196-201 | | | | | | | | |
| 56 | TIC807_56 | F46S, Y54H, F147A, | F147A | S217N, then a | ND | — | 64 | ND | — | ND |
| | | S167R, S217N, | | contiguous triple | | | | | | |
| | | R282A, then a | | deletion in residue | | | | | | |
| | | contiguous triple | | range 196-201 | | | | | | |
| | | deletion in residue | | | | | | | | |
| | | range 196-201 | | | | | | | | |
| 57 | TIC807_57 | F46S, Y54H, T93A, | T93A | S217N, then a | ND | — | 63 | ND | — | ND |
| | | S167R, S217N, | | contiguous triple | | | | | | |
| | | T308A, then a | | deletion in residue | | | | | | |
| | | contiguous triple | | range 196-201 | | | | | | |
| | | deletion in residue | | | | | | | | |
| | | range 196-201 | | | | | | | | |
| 58 | TIC807_58 | F46S, Y54H, Q70A, | T93A | S217N, then a | ND | — | 61 | ND | — | ND |
| | | T93A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 59 | TIC807_59 | F46S, Y54H, E125A, | F147A | S217N, then a | ND | — | 61 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 60 | TIC807_60 | F46S, Y54H, F147A, | F147A | S217N, then a | ND | — | 61 | ND | — | ND |
| | | S167R, S217N, | | contiguous triple | | | | | | |
| | | T247A, then a | | deletion in residue | | | | | | |
| | | contiguous triple | | range 196-201 | | | | | | |
| | | deletion in residue | | | | | | | | |
| | | range 196-201 | | | | | | | | |
| 61 | TIC807_61 | F46S, Y54H, T93A, | T93A | S217N, then a | ND | — | 59 | ND | — | ND |
| | | S167R, S217N, | | contiguous triple | | | | | | |
| | | P305A, then a | | deletion in residue | | | | | | |
| | | contiguous triple | | range 196-201 | | | | | | |
| | | deletion in residue | | | | | | | | |
| | | range 196-201 | | | | | | | | |
| 62 | TIC807_62 | F46S, Y54H, F147A, | F147A | S217N, then a | ND | — | 57 | ND | — | ND |
| | | S167R, S217N, | | contiguous triple | | | | | | |
| | | I306A, then a | | deletion in residue | | | | | | |
| | | contiguous triple | | range 196-201 | | | | | | |
| | | deletion in residue | | | | | | | | |
| | | range 196-201 | | | | | | | | |
| 63 | TIC807_63 | F46S, Y54H, T93A, | T93A | S217N, then a | ND | — | 56 | ND | — | ND |
| | | S167R, S217N, | | contiguous triple | | | | | | |
| | | R282A, then a | | deletion in residue | | | | | | |
| | | contiguous triple | | range 196-201 | | | | | | |
| | | deletion in residue | | | | | | | | |
| | | range 196-201 | | | | | | | | |
| 64 | TIC807_64 | F46S, Y54H, T93A, | T93A | S217N, then a | ND | — | 55 | ND | — | ND |
| | | S167R, S217N, | | contiguous triple | | | | | | |
| | | T308A, then a | | deletion in residue | | | | | | |
| | | contiguous triple | | range 196-201 | | | | | | |
| | | deletion in residue | | | | | | | | |
| | | range 196-201 | | | | | | | | |
| 65 | TIC807_65 | F46S, Y54H, T93A, | T93A | S217N, then a | ND | — | 55 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 66 | TIC807_66 | F46S, Y54H, T93A, | T93A | S217N, then a | ND | — | 53 | ND | — | ND |
| | | S167R, S217N, | | contiguous triple | | | | | | |
| | | H287A, then a | | deletion in residue | | | | | | |
| | | contiguous triple | | range 196-201 | | | | | | |
| | | deletion in residue | | | | | | | | |
| | | range 196-201 | | | | | | | | |
| 67 | TIC807_67 | F46S, Y54H, S95A, | S95A | S217N, then a | ND | — | 52 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 68 | TIC807_68 | F46S, Y54H, F147A, | F147A | S217N, then a | ND | — | 52 | ND | — | ND |
| | | S167R, S217N, | | contiguous triple | | | | | | |
| | | I275A, then a | | deletion in residue | | | | | | |
| | | contiguous triple | | range 196-201 | | | | | | |
| | | deletion in residue | | | | | | | | |
| | | range 196-201 | | | | | | | | |
| 69 | TIC807_69 | F46S, Y54H, S95A, | S95A | S217N, then a | ND | — | 51 | ND | — | ND |
| | | S167R, S217N, | | contiguous triple | | | | | | |
| | | T247A, then a | | deletion in residue | | | | | | |
| | | contiguous triple | | range 196-201 | | | | | | |
| | | deletion in residue | | | | | | | | |
| | | range 196-201 | | | | | | | | |
| 70 | TIC807_70 | F46S, Y54H, F147A, | F147A | S217N, then a | ND | — | 50 | ND | — | ND |
| | | S167R, V175A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 71 | TIC807_71 | F46S, Y54H, F147A, | F147A | S217N, then a | ND | — | 50 | ND | — | ND |
| | | S159A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 72 | TIC807_72 | F46S, Y54H, S95A, | S95A, F147A | S217N, then a | ND | — | 48 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 73 | TIC807_73 | F46S, Y54H, F147A, | F147A | S217N, then a | ND | — | 47 | ND | — | ND |
| | | S167R, L187A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 74 | TIC807_74 | F46S, Y54H, T93A, | T93A | S217N, then a | ND | — | 47 | ND | — | ND |
| | | S167R, T182A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 75 | TIC807_75 | F46S, Y54H, F147A, | F147A | S217N, then a | ND | — | 46 | ND | — | ND |
| | | S167R, S217N, | | contiguous triple | | | | | | |
| | | T245A, then a | | deletion in residue | | | | | | |
| | | contiguous triple | | range 196-201 | | | | | | |
| | | deletion in residue | | | | | | | | |
| | | range 196-201 | | | | | | | | |
| 76 | TIC807_76 | F46S, Y54H, T93A, | T93A | S217N, then a | ND | — | 46 | ND | — | ND |
| | | S167R, S217N, | | contiguous triple | | | | | | |
| | | S249A, then a | | deletion in residue | | | | | | |
| | | contiguous triple | | range 196-201 | | | | | | |
| | | deletion in residue | | | | | | | | |
| | | range 196-201 | | | | | | | | |
| 77 | TIC807_77 | F46S, Y54H, T93A, | T93A, Q149A | S217N, then a | ND | — | 45 | ND | — | ND |
| | | Q149A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 78 | TIC807_78 | F46S, Y54H, T93A, | T93A, S151A | S217N, then a | ND | — | 45 | ND | — | ND |
| | | S151A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 79 | TIC807_79 | F46S, Y54H, Q70A, | F147A | S217N, then a | ND | — | 45 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 80 | TIC807_80 | F46S, Y54H, S95A, | S95A | S217N, then a | ND | — | 44 | ND | — | ND |
| | | Q148A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 81 | TIC807_81 | F46S, Y54H, T93A, | T93A | S217N, then a | ND | — | 44 | ND | — | ND |
| | | S167R, S217N, | | contiguous triple | | | | | | |
| | | T274A, then a | | deletion in residue | | | | | | |
| | | contiguous triple | | range 196-201 | | | | | | |
| | | deletion in residue | | | | | | | | |
| | | range 196-201 | | | | | | | | |
| 82 | TIC807_82 | F46S, Y54H, | T93A | S217N, then a | ND | — | 43 | ND | — | ND |
| | | T93A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 83 | TIC807_83 | F46S, Y54H, T93A, | T93A | S217N, then a | ND | — | 42 | ND | — | ND |
| | | S167R, M177A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 84 | TIC807_84 | F46S, Y54H, S95A, | S95A | S217N, then a | ND | — | 42 | ND | — | ND |
| | | S167R, S217N, | | contiguous triple | | | | | | |
| | | V250A, then a | | deletion in residue | | | | | | |
| | | contiguous triple | | range 196-201 | | | | | | |
| | | deletion in residue | | | | | | | | |
| | | range 196-201 | | | | | | | | |
| 85 | TIC807_85 | F46S, Y54H, T93A, | T93A | S217N, then a | ND | — | 42 | ND | — | ND |
| | | E155A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 86 | TIC807_86 | F46S, Y54H, I134A, | F147A | S217N, then a | ND | — | 41 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 87 | TIC807_87 | F46S, Y54H, T93A, | T93A | S217N, then a | ND | — | 41 | ND | — | ND |
| | | S167R, S217N, | | contiguous triple | | | | | | |
| | | T245A, then a | | deletion in residue | | | | | | |
| | | contiguous triple | | range 196-201 | | | | | | |
| | | deletion in residue | | | | | | | | |
| | | range 196-201 | | | | | | | | |
| 88 | TIC807_88 | F46S, Y54H, T93A, | T93A | S217N, then a | ND | — | 40 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by S198A | | | | | | |
| | | followed by a S198A | | | | | | | | |
| 89 | TIC807_89 | F46S, Y54H, F147A, | F147A | S217N, then a | ND | — | 40 | ND | — | ND |
| | | S167R, S217N, | | contiguous triple | | | | | | |
| | | N295A, then a | | deletion in residue | | | | | | |
| | | contiguous triple | | range 196-201 | | | | | | |
| | | deletion in residue | | | | | | | | |
| | | range 196-201 | | | | | | | | |
| 90 | TIC807_90 | F46S, Y54H, A87S, | F147A | S217N, then a | ND | — | 38 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 91 | TIC807_91 | F46S, Y54H, F147A, | F147A | S217N, then a | ND | — | 38 | ND | — | ND |
| | | S167R, S217N, | | contiguous triple | | | | | | |
| | | S249A, then a | | deletion in residue | | | | | | |
| | | contiguous triple | | range 196-201 | | | | | | |
| | | deletion in residue | | | | | | | | |
| | | range 196-201 | | | | | | | | |
| 92 | TIC807_92 | F46S, Y54H, S95A, | S95A | S217N, then a | ND | — | 37 | ND | — | ND |
| | | S167R, S217N, | | contiguous triple | | | | | | |
| | | I275A, then a | | deletion in residue | | | | | | |
| | | contiguous triple | | range 196-201 | | | | | | |
| | | deletion in residue | | | | | | | | |
| | | range 196-201 | | | | | | | | |
| 93 | TIC807_93 | F46S, Y54H, T93A, | T93A | S217N, then a | ND | — | 37 | ND | — | ND |
| | | A139S, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 94 | TIC807_94 | F46S, Y54H, F147A, | F147A | S217N, then a | ND | — | 36 | ND | — | ND |
| | | S167R, S217N, | | contiguous triple | | | | | | |
| | | P305A, then a | | deletion in residue | | | | | | |
| | | contiguous triple | | range 196-201 | | | | | | |
| | | deletion in residue | | | | | | | | |
| | | range 196-201 | | | | | | | | |
| 95 | TIC807_95 | F46S, Y54H, F147A, | F147A | S217N, then a | ND | — | 36 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by S198A | | | | | | |
| | | followed by a S198A | | | | | | | | |
| 96 | TIC807_96 | F46S, Y54H, F147A, | F147A | S217N, then a | ND | — | 36 | ND | — | ND |
| | | S167R, S217N, | | contiguous triple | | | | | | |
| | | S252A, then a | | deletion in residue | | | | | | |
| | | contiguous triple | | range 196-201 | | | | | | |
| | | deletion in residue | | | | | | | | |
| | | range 196-201 | | | | | | | | |
| 97 | TIC807_97 | F46S, Y54H, T93A, | T93A, F147A | S217N, then a | ND | — | 36 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 98 | TIC807_98 | F46S, Y54H, F147A, | F147A | S217N, then a | ND | — | 36 | ND | — | ND |
| | | S167R, S217N, | | contiguous triple | | | | | | |
| | | V250A, then a | | deletion in residue | | | | | | |
| | | contiguous triple | | range 196-201 | | | | | | |
| | | deletion in residue | | | | | | | | |
| | | range 196-201 | | | | | | | | |
| 99 | TIC807_99 | F46S, Y54H, F147A, | F147A | S217N, then a | ND | — | 35 | ND | — | ND |
| | | S167R, S217N, | | contiguous triple | | | | | | |
| | | T243A, then a | | deletion in residue | | | | | | |
| | | contiguous triple | | range 196-201 | | | | | | |
| | | deletion in residue | | | | | | | | |
| | | range 196-201 | | | | | | | | |
| 100 | TIC807_100 | F46S, Y54H, F147A, | F147A | S217N, then a | ND | — | 35 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by Y197A | | | | | | |
| | | followed by a Y197A | | | | | | | | |
| 101 | TIC807_101 | F46S, Y54H, S95A, | S95A | S217N, then a | ND | — | 35 | ND | — | ND |
| | | S167R, S217N, | | contiguous triple | | | | | | |
| | | N295A, then a | | deletion in residue | | | | | | |
| | | contiguous triple | | range 196-201 | | | | | | |
| | | deletion in residue | | | | | | | | |
| | | range 196-201 | | | | | | | | |
| 102 | TIC807_102 | F46S, Y54H, F147A, | F147A | S217N, then a | ND | — | 34 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 103 | TIC807_103 | F46S, Y54H, F147A, | F147A | S217N, then a | ND | — | 32 | ND | — | ND |
| | | S167R, S217N, | | contiguous triple | | | | | | |
| | | E299A, then a | | deletion in residue | | | | | | |
| | | contiguous triple | | range 196-201 | | | | | | |
| | | deletion in residue | | | | | | | | |
| | | range 196-201 | | | | | | | | |
| 104 | TIC807_104 | F46S, Y54H, S95A, | S95A | S217N, then a | ND | — | 32 | ND | — | ND |
| | | S167R, S217N, | | contiguous triple | | | | | | |
| | | R282A, then a | | deletion in residue | | | | | | |
| | | contiguous triple | | range 196-201 | | | | | | |
| | | deletion in residue | | | | | | | | |
| | | range 196-201 | | | | | | | | |
| 105 | TIC807_105 | F46S, Y54H, T93A, | T93A | S217N, then a | ND | — | 32 | ND | — | ND |
| | | S167R, S217N, | | contiguous triple | | | | | | |
| | | I306A, then a | | deletion in residue | | | | | | |
| | | contiguous triple | | range 196-201 | | | | | | |
| | | deletion in residue | | | | | | | | |
| | | range 196-201 | | | | | | | | |
| 106 | TIC807_106 | F46S, Y54H, S95A, | S95A | S217N, then a | ND | — | 30 | ND | — | ND |
| | | S167R, S217N, | | contiguous triple | | | | | | |
| | | S249A, then a | | deletion in residue | | | | | | |
| | | contiguous triple | | range 196-201 | | | | | | |
| | | deletion in residue | | | | | | | | |
| | | range 196-201 | | | | | | | | |
| 107 | TIC807_107 | F46S, Y54H, A87S, | T93A | S217N, then a | ND | — | 30 | ND | — | ND |
| | | T93A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 108 | TIC807_108 | F46S, Y54H, T93A, | T93A | S217N, then a | ND | — | 29 | ND | — | ND |
| | | S159A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 109 | TIC807_109 | F46S, Y54H, F147A, | F147A | S217N, then a | ND | — | 28 | ND | — | ND |
| | | S167R, S217N, | | contiguous triple | | | | | | |
| | | T303A, then a | | deletion in residue | | | | | | |
| | | contiguous triple | | range 196-201 | | | | | | |
| | | deletion in residue | | | | | | | | |
| | | range 196-201 | | | | | | | | |
| 110 | TIC807_110 | F46S, Y54H, T93A, | T93A | S217N, then a | ND | — | 27 | ND | — | ND |
| | | Q148A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 111 | TIC807_111 | F46S, Y54H, S95A, | S95A | S217N, then a | ND | — | 27 | ND | — | ND |
| | | S167R, V175N, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 112 | TIC807_112 | F46S, Y54H, F147A, | F147A | S217N, then a | ND | — | 27 | ND | — | ND |
| | | S167R, S217N, | | contiguous triple | | | | | | |
| | | D241A, then a | | deletion in residue | | | | | | |
| | | contiguous triple | | range 196-201 | | | | | | |
| | | deletion in residue | | | | | | | | |
| | | range 196-201 | | | | | | | | |
| 113 | TIC807_113 | F46S, Y54H, S95A, | S95A, F147A | S217N, then a | ND | — | 26 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 114 | TIC807_114 | F46S, Y54H, F147A, | F147A | S217N, then a | ND | — | 26 | ND | — | ND |
| | | S167R, M177A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 115 | TIC807_115 | F46S, Y54H, F147A, | F147A | S217N, then a | ND | — | 26 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 116 | TIC807_116 | F46S, Y54H, S95A, | S95A | S217N, then a | ND | — | 26 | ND | — | ND |
| | | S167R, S217N, | | contiguous triple | | | | | | |
| | | P305A, then a | | deletion in residue | | | | | | |
| | | contiguous triple | | range 196-201 | | | | | | |
| | | deletion in residue | | | | | | | | |
| | | range 196-201 | | | | | | | | |
| 117 | TIC807_117 | F46S, Y54H, F147A, | F147A | S217N, then a | ND | — | 25 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by H196A | | | | | | |
| | | followed by H196A | | | | | | | | |
| 118 | TIC807_118 | F46S, Y54H, A139S, | F147A | S217N, then a | ND | — | 25 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 119 | TIC807_119 | F46S, Y54H, T93A, | T93A | S217N, then a | ND | — | 25 | ND | — | ND |
| | | S167R, S217N, | | contiguous triple | | | | | | |
| | | N295A, then a | | deletion in residue | | | | | | |
| | | contiguous triple | | range 196-201 | | | | | | |
| | | deletion in residue | | | | | | | | |
| | | range 196-201 | | | | | | | | |
| 120 | TIC807_120 | F46S, Y54H, T93A, | T93A | S217N, then a | ND | — | 24 | ND | — | ND |
| | | T145A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 121 | TIC807_121 | F46S, Y54H, S117A, | F147A | S217N, then a | ND | — | 24 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 122 | TIC807_122 | F46S, Y54H, T93A, | T93A | S217N, then a | ND | — | 23 | ND | — | ND |
| | | S167R, S217N, | | contiguous triple | | | | | | |
| | | I275A, then a | | deletion in residue | | | | | | |
| | | contiguous triple | | range 196-201 | | | | | | |
| | | deletion in residue | | | | | | | | |
| | | range 196-201 | | | | | | | | |
| 123 | TIC807_123 | F46S, Y54H, S95A, | S95A | S217N, then a | ND | — | 23 | ND | — | ND |
| | | S167R, S217N, | | contiguous triple | | | | | | |
| | | H287A, then a | | deletion in residue | | | | | | |
| | | contiguous triple | | range 196-201 | | | | | | |
| | | deletion in residue | | | | | | | | |
| | | range 196-201 | | | | | | | | |
| 124 | TIC807_124 | F46S, Y54H, S95A, | S95A | S217N, then a | ND | — | 21 | ND | — | ND |
| | | G105A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 125 | TIC807_125 | F46S, Y54H, S95A, | S95A | S217N, then a | ND | — | 20 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 126 | TIC807_126 | F46S, Y54H, S95A, | S95A | S217N, then a | ND | — | 20 | ND | — | ND |
| | | I134A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 127 | TIC807_127 | F46S, Y54H, T93A, | T93A | S217N, then a | ND | — | 20 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 128 | TIC807_128 | F46S, Y54H, T93A, | T93A | S217N, then a | ND | — | 18 | ND | — | ND |
| | | S167R, S217N, | | contiguous triple | | | | | | |
| | | T303A, then a | | deletion in residue | | | | | | |
| | | contiguous triple | | range 196-201 | | | | | | |
| | | deletion in residue | | | | | | | | |
| | | range 196-201 | | | | | | | | |
| 129 | TIC807_129 | F46S, Y54H, T93A, | T93A | S217N, then a | ND | — | 18 | ND | — | ND |
| | | A150S, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 130 | TIC807_130 | F46S, Y54H, S95A, | S95A | S217N, then a | ND | — | 18 | ND | — | ND |
| | | E155A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 131 | TIC807_131 | F46S, Y54H, T93A, | T93A | S217N, then a | ND | — | 17 | ND | — | ND |
| | | T145A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 132 | TIC807_132 | F46S, Y54H, S95A, | S95A | W208A, S217N, | ND | — | 17 | ND | — | ND |
| | | S167R, W208A, | | then a contiguous | | | | | | |
| | | S217N, then a | | triple deletion in | | | | | | |
| | | contiguous triple | | residue | | | | | | |
| | | deletion in residue | | range 196-201 | | | | | | |
| | | range 196-201 | | | | | | | | |
| 133 | TIC807_133 | F46S, Y54H, S95A, | S95A, T182A | S217N, then a | ND | — | 17 | ND | — | ND |
| | | S167R, T182A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 134 | TIC807_134 | F46S, Y54H, T93A, | T93A | S217N, then a | ND | — | 17 | ND | — | ND |
| | | S167R, S217N, | | contiguous triple | | | | | | |
| | | T243A, then a | | deletion in residue | | | | | | |
| | | contiguous triple | | range 196-201 | | | | | | |
| | | deletion in residue | | | | | | | | |
| | | range 196-201 | | | | | | | | |
| 135 | TIC807_135 | F46S, Y54H, S95A, | S95A | S217N, then a | ND | — | 16 | ND | — | ND |
| | | S167R, S217N, | | contiguous triple | | | | | | |
| | | I306A, then a | | deletion in residue | | | | | | |
| | | contiguous triple | | range 196-201 | | | | | | |
| | | deletion in residue | | | | | | | | |
| | | range 196-201 | | | | | | | | |
| 136 | TIC807_136 | F46S, Y54H, S95A, | S95A | S217N, then a | ND | — | 16 | ND | — | ND |
| | | S117A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 137 | TIC807_137 | F46S, Y54H, T93A, | T93A | S217N, then a | ND | — | 15 | ND | — | ND |
| | | S119A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 138 | TIC807_138 | F46S, Y54H, T68A, | F147A | S217N, then a | ND | — | 15 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 139 | TIC807_139 | F46S, Y54H, G105A, | F147A | S217N, then a | ND | — | 15 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 140 | TIC807_140 | F46S, Y54H, S95A, | S95A | S217N, then a | ND | — | 15 | ND | — | ND |
| | | E125A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 141 | TIC807_141 | F46S, Y54H, T93A, | T93A | S217N, then a | ND | — | 14 | ND | — | ND |
| | | E155A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 142 | TIC807_142 | F46S, Y54H, T93A, | T93A | S217N, then a | ND | — | 14 | ND | — | ND |
| | | S167R, S217N, | | contiguous triple | | | | | | |
| | | P305A, then a | | deletion in residue | | | | | | |
| | | contiguous triple | | range 196-201 | | | | | | |
| | | deletion in residue | | | | | | | | |
| | | range 196-201 | | | | | | | | |
| 143 | TIC807_143 | F46S, Y54H, S95A, | S95A | S217N, then a | ND | — | 13 | ND | — | ND |
| | | S167R, M177A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 144 | TIC807_144 | F46S, Y54H, T93A, | T93A | S217N, then a | ND | — | 13 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by H196A | | | | | | |
| | | followed by H196A | | | | | | | | |
| 145 | TIC807_145 | F46S, Y54H, T93A, | T93A | S217N, then a | ND | — | 11 | ND | — | ND |
| | | D153A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201-T93A- | | | | | | | | |
| | | D153A | | | | | | | | |
| 146 | TIC807_146 | F46S, Y54H, F147A, | F147A | W208A, S217N, | ND | — | 10 | ND | — | ND |
| | | S167R, S217N, | | then a contiguous | | | | | | |
| | | W208A, then a | | triple deletion in | | | | | | |
| | | contiguous triple | | residue | | | | | | |
| | | deletion in residue | | range 196-201 | | | | | | |
| | | range 196-201 | | | | | | | | |
| 147 | TIC807_147 | F46S, Y54H, T93A, | T93A | S217N, then a | ND | — | 9 | ND | — | ND |
| | | S167R, S217N, | | contiguous triple | | | | | | |
| | | I306A, then a | | deletion in residue | | | | | | |
| | | contiguous triple | | range 196-201 | | | | | | |
| | | deletion in residue | | | | | | | | |
| | | range 196-201 | | | | | | | | |
| 148 | TIC807_148 | F46S, Y54H, S95A, | S95A | S217N, then a | ND | — | 9 | ND | — | ND |
| | | S167R, S217N, then a | | contiguous triple | | | | | | |
| | | contiguous triple | | deletion in residue | | | | | | |
| | | deletion in residue | | range 196-201 | | | | | | |
| | | range 196-201 | | followed by H196A | | | | | | |
| | | followed by H196A | | | | | | | | |
| 149 | TIC807_149 | F46S, Y54H, S95A, | S95A, Q149A | S217N, then a | ND | — | 9 | ND | — | ND |
| | | Q149A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 150 | TIC807_150 | F46S, Y54H, S95A, | S95A | S217N, then a | ND | — | 8 | ND | — | ND |
| | | S167R, S217N, | | contiguous triple | | | | | | |
| | | S293A, then a | | deletion in residue | | | | | | |
| | | contiguous triple | | range 196-201 | | | | | | |
| | | deletion in residue | | | | | | | | |
| | | range 196-201 | | | | | | | | |
| 151 | TIC807_151 | F46S, Y54H, S95A, | S95A | S217N, then a | ND | — | 8 | ND | — | ND |
| | | A150S, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 152 | TIC807_152 | F46S, Y54H, S119A, | F147A | S217N, then a | ND | — | 7 | ND | — | ND |
| | | F147A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 153 | TIC807_153 | F46S, Y54H, S95A, | S95A | S217N, then a | ND | — | 7 | ND | — | ND |
| | | S167A, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 154 | TIC807_154 | F46S, Y54H, F147A, | F147A | S217N, then a | ND | — | 7 | ND | — | ND |
| | | I158A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 155 | TIC807_155 | F46S, Y54H, S95A, | S95A | S217N, then a | ND | — | 7 | ND | — | ND |
| | | S167R, S217N, | | contiguous triple | | | | | | |
| | | M300A, then a | | deletion in residue | | | | | | |
| | | contiguous triple | | range 196-201 | | | | | | |
| | | deletion in residue | | | | | | | | |
| | | range 196-201 | | | | | | | | |
| 156 | TIC807_156 | F46S, Y54H, F147A, | F147A, T182A | S217N, then a | ND | — | 6 | ND | — | ND |
| | | T182A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 157 | TIC807_157 | F46S, Y54H, F147A, | F147A | S217N, then a | ND | — | 5 | ND | — | ND |
| | | S167R, S217N, | | contiguous triple | | | | | | |
| | | F235A, then a | | deletion in residue | | | | | | |
| | | contiguous triple | | range 196-201 | | | | | | |
| | | deletion in residue | | | | | | | | |
| | | range 196-201 | | | | | | | | |
| 158 | TIC807_158 | F46S, Y54H, F147A, | F147A | S217N, then a | ND | — | 5 | ND | — | ND |
| | | S167A, S217N, then a | | contiguous triple | | | | | | |
| | | contiguous triple | | deletion in residue | | | | | | |
| | | deletion in residue | | range 196-201 | | | | | | |
| | | range 196-201 | | | | | | | | |
| 159 | TIC807_159 | F46S, Y54H, T93A, | T93A | S217N, then a | ND | — | 5 | ND | — | ND |
| | | S167R, S217N, | | contiguous triple | | | | | | |
| | | D241A, then a | | deletion in residue | | | | | | |
| | | contiguous triple | | range 196-201 | | | | | | |
| | | deletion in residue | | | | | | | | |
| | | range 196-201 | | | | | | | | |
| 160 | TIC807_160 | F46S, Y54H, S95A, | S95A | S217N, then a | ND | — | 4 | ND | — | ND |
| | | S167R, S217N, | | contiguous triple | | | | | | |
| | | T274A, then a | | deletion in residue | | | | | | |
| | | contiguous triple | | range 196-201 | | | | | | |
| | | deletion in residue | | | | | | | | |
| | | range 196-201 | | | | | | | | |
| 161 | TIC807_161 | F46S, Y54H, T93A, | T93A | S217N, then a | ND | — | 4 | ND | — | ND |
| | | Q148A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 162 | TIC807_162 | F46S, Y54H, S95A, | S95A | S217N, then a | ND | — | 4 | ND | — | ND |
| | | S167R, S217N, | | contiguous triple | | | | | | |
| | | D241A, then a | | deletion in residue | | | | | | |
| | | contiguous triple | | range 196-201 | | | | | | |
| | | deletion in residue | | | | | | | | |
| | | range 196-201 | | | | | | | | |
| 163 | TIC807_163 | F46S, Y54H, S95A, | S95A | S217N, then a | ND | — | 4 | ND | — | ND |
| | | E155A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 164 | TIC807_164 | F46S, Y54H, T93A, | T93A | S217N, then a | ND | — | 4 | ND | — | ND |
| | | S167R, L187A, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 165 | TIC807_165 | F46S, Y54H, T93A, | T93A | S217N, then a | ND | — | 4 | ND | — | ND |
| | | S167R, S217N, | | contiguous triple | | | | | | |
| | | T303A, then a | | deletion in residue | | | | | | |
| | | contiguous triple | | range 196-201 | | | | | | |
| | | deletion in residue | | | | | | | | |
| | | range 196-201 | | | | | | | | |
| 166 | TIC807_166 | F46S, Y54H, T93A, | T93A | S217N, then a | ND | — | 3 | ND | — | ND |
| | | E155A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 167 | TIC807_167 | F46S, Y54H, T93A, | T93A | S217N, then a | ND | — | 3 | ND | — | ND |
| | | D153A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 168 | TIC807_168 | F46S, Y54H, T93A, | T93A | S217N, then a | ND | — | 3 | ND | — | ND |
| | | I134A, S167R, | | contiguous triple | | | | | | |
| | | S217N, then a | | deletion in residue | | | | | | |
| | | contiguous triple | | range 196-201 | | | | | | |
| | | deletion in residue | | | | | | | | |
| | | range 196-201 | | | | | | | | |
| 169 | TIC807_169 | N137T | none | None | ND | — | 44* | ND | — | ND |
| 169 | TIC807_169 | E135S | none | None | ND | — | 20* | ND | — | ND |
| 169 | TIC807_169 | N137T, E135S | none | None | ND | — | 34* | ND | — | ND |
| 169 | TIC807_169 | E135T, N137D | none | None | ND | — | 16* | ND | — | ND |
| 169 | TIC807_169 | T133E | none | None | ND | — | 60* | ND | — | ND |
| 169 | TIC807_169 | E135A, N137G | none | None | ND | — | 50* | ND | — | ND |
| 170 | TIC807_170 | E125C | none | None | ND | — | 12 | ND | — | ND |
| 170 | TIC807_170 | E125H | none | None | ND | — | 38 | ND | — | ND |
| 170 | TIC807_170 | E125R | none | None | ND | — | 14 | ND | — | ND |
| 170 | TIC807_170 | E125F | none | None | ND | — | 33 | ND | — | ND |
| 170 | TIC807_170 | E125S | none | None | ND | — | 24 | ND | — | ND |
| 170 | TIC807_170 | E125Q | none | None | ND | — | 21 | ND | — | ND |
| 170 | TIC807_170 | E125K | none | None | ND | — | 20 | ND | — | ND |
| 170 | TIC807_170 | E125T | none | None | ND | — | 33 | ND | — | ND |
| 170 | TIC807_170 | E125N | none | None | ND | — | 19 | ND | — | ND |
| 170 | TIC807_170 | E125A | none | None | ND | — | 41 | ND | — | ND |
| 170 | TIC807_170 | E125L | none | None | ND | — | 13 | ND | — | ND |
| 170 | TIC807_170 | E125V | none | None | ND | — | 14 | ND | — | ND |
| 170 | TIC807_170 | E125M | none | None | ND | — | 13 | ND | — | ND |
| 170 | TIC807_170 | E125D | none | None | ND | — | 15 | ND | — | ND |
| 170 | TIC807_170 | E125Y | none | None | ND | — | 38 | ND | — | ND |
| 171 | TIC807_171 | T133E | none | None | ND | — | 23 | ND | — | ND |
| 171 | TIC807_171 | T133Y | none | None | ND | — | 17 | ND | — | ND |
| 171 | TIC807_171 | T133W | none | None | ND | — | 13 | ND | — | ND |
| 172 | TIC807_172 | I134V | none | None | ND | — | 18 | ND | — | ND |
| 172 | TIC807_172 | I134L | none | None | ND | — | 10 | ND | — | ND |
| 172 | TIC807_172 | I134F | none | None | ND | — | 18 | ND | — | ND |
| 172 | TIC807_172 | I134K | none | None | ND | — | 13 | ND | — | ND |
| 172 | TIC807_172 | I134C | none | None | ND | — | 30 | ND | — | ND |
| 172 | TIC807_172 | I134M | none | None | ND | — | 33 | ND | — | ND |
| 173 | TIC807_173 | E135V | none | None | ND | — | 13 | ND | — | ND |
| 173 | TIC807_173 | E135W | none | None | ND | — | 13 | ND | — | ND |
| 173 | TIC807_173 | E135T | none | None | ND | — | 39 | ND | — | ND |
| 174 | TIC807_174 | N137H | none | None | ND | — | 42 | ND | — | ND |
| 174 | TIC807_174 | N137Y | none | None | ND | — | 17 | ND | — | ND |
| 174 | TIC807_174 | N137T | none | None | ND | — | 31 | ND | — | ND |
| 174 | TIC807_174 | N137E | none | None | ND | — | 32 | ND | — | ND |
| 174 | TIC807_174 | N137S | none | None | ND | — | 24 | ND | — | ND |
| 174 | TIC807_174 | N137A | none | None | ND | — | 24 | ND | — | ND |
| 174 | TIC807_174 | N137Q | none | None | ND | — | 21 | ND | — | ND |
| 174 | TIC807_174 | N137G | none | None | ND | — | 18 | ND | — | ND |
| 174 | TIC807_174 | N137I | none | None | ND | — | 10 | ND | — | ND |
| 174 | TIC807_174 | N137W | none | None | ND | — | 17 | ND | — | ND |
| 174 | TIC807_174 | N137K | none | None | ND | — | 66 | ND | — | ND |
| 174 | TIC807_174 | N137C | none | None | ND | — | 19 | ND | — | ND |
| 174 | TIC807_174 | N137M | none | None | ND | — | 41 | ND | — | ND |
| 174 | TIC807_174 | N137D | none | None | ND | — | 69 | ND | — | ND |
| 174 | TIC807_174 | N137F | none | None | ND | — | 13 | ND | — | ND |
| 174 | TIC807_174 | N137R | none | None | ND | — | 37 | ND | — | ND |
| 175 | TIC807_175 | F147V | F147V | None | ND | — | 87 | ND | — | ND |
| 175 | TIC807_175 | F147T | F147T | None | ND | — | 68 | ND | — | ND |
| 175 | TIC807_175 | F147C | F147C | None | ND | — | 74 | ND | — | ND |
| 175 | TIC807_175 | F147L | F147L | None | ND | — | 62 | ND | — | ND |
| 175 | TIC807_175 | F147D | F147D | None | ND | — | 51 | ND | — | ND |
| 175 | TIC807_175 | F147A | F147A | None | ND | — | 57 | ND | — | ND |
| 175 | TIC807_175 | F147G | F147G | None | ND | — | 56 | ND | — | ND |
| 175 | TIC807_175 | F147E | F147E | None | ND | — | 50 | ND | — | ND |
| 175 | TIC807_175 | F147I | F147I | None | ND | — | 69 | ND | — | ND |
| 175 | TIC807_175 | F147Y | F147Y | None | ND | — | 67 | ND | — | ND |
| 175 | TIC807_175 | F147M | F147M | None | ND | — | 64 | ND | — | ND |
| 175 | TIC807_175 | F147N | F147N | None | ND | — | 64 | ND | — | ND |
| 175 | TIC807_175 | F147Q | F147Q | None | ND | — | 50 | ND | — | ND |
| 175 | TIC807_175 | F147H | F147H | None | ND | — | 60 | ND | — | ND |
| 175 | TIC807_175 | F147R | F147R | None | ND | — | 20 | ND | — | ND |
| 175 | TIC807_175 | F147W | F147W | None | ND | — | 82 | ND | — | ND |
| 175 | TIC807_175 | F147P | F147P | None | ND | — | 7 | ND | — | ND |
| 176 | TIC807_176 | Q149D | Q149D | None | ND | — | 92 | ND | — | ND |
| 176 | TIC807_176 | Q149E | Q149E | None | ND | — | 89 | ND | — | ND |
| 176 | TIC807_176 | Q149C | Q149C | None | ND | — | 87 | ND | — | ND |
| 176 | TIC807_176 | Q149A | Q149A | None | ND | — | 76 | ND | — | ND |
| 176 | TIC807_176 | Q149F | Q149F | None | ND | — | 54 | ND | — | ND |
| 177 | TIC807_177 | A150S | none | None | ND | — | 34 | ND | — | ND |
| 177 | TIC807_177 | A150L | none | None | ND | — | 24 | ND | — | ND |
| 177 | TIC807_177 | A150V | none | None | ND | — | 25 | ND | — | ND |
| 177 | TIC807_177 | A150G | none | None | ND | — | 28 | ND | — | ND |
| 177 | TIC807_177 | A150D | none | None | ND | — | 19 | ND | — | ND |
| 177 | TIC807_177 | A150W | none | None | ND | — | 13 | ND | — | ND |
| 177 | TIC807_177 | A150E | none | None | ND | — | 24 | ND | — | ND |
| 177 | TIC807_177 | A150N | none | None | ND | — | 18 | ND | — | ND |
| 177 | TIC807_177 | A150Y | none | None | ND | — | 11 | ND | — | ND |
| 177 | TIC807_177 | A150F | none | None | ND | — | 11 | ND | — | ND |
| 177 | TIC807_177 | A150P | none | None | ND | — | 11 | ND | — | ND |
| 177 | TIC807_177 | A150K | none | None | ND | — | 17 | ND | — | ND |
| 177 | TIC807_177 | A150T | none | None | ND | — | 17 | ND | — | ND |
| 177 | TIC807_177 | A150Q | none | None | ND | — | 11 | ND | — | ND |
| 177 | TIC807_177 | A150R | none | None | ND | — | 11 | ND | — | ND |
| 178 | TIC807_178 | E155C | none | None | ND | — | 82 | ND | — | ND |
| 178 | TIC807_178 | E155I | none | None | ND | — | 36 | ND | — | ND |
| 178 | TIC807_178 | E155K | none | None | ND | — | 28 | ND | — | ND |
| 178 | TIC807_178 | E155D | none | None | ND | — | 22 | ND | — | ND |
| 178 | TIC807_178 | E155H | none | None | ND | — | 22 | ND | — | ND |
| 178 | TIC807_178 | E155Y | none | None | ND | — | 16 | ND | — | ND |
| 178 | TIC807_178 | E155Q | none | None | ND | — | 16 | ND | — | ND |
| 178 | TIC807_178 | E155L | none | None | ND | — | 15 | ND | — | ND |
| 178 | TIC807_178 | E155N | none | None | ND | — | 14 | ND | — | ND |
| 178 | TIC807_178 | E155T | none | None | ND | — | 13 | ND | — | ND |
| 178 | TIC807_178 | E155A | none | None | ND | — | 11 | ND | — | ND |
| 178 | TIC807_178 | E155F | none | None | ND | — | 7 | ND | — | ND |
| 178 | TIC807_178 | E155R | none | None | ND | — | 6 | ND | — | ND |
| 178 | TIC807_178 | E155M | none | None | ND | — | 6 | ND | — | ND |
| 178 | TIC807_178 | E155P | none | None | ND | — | 5 | ND | — | ND |
| 178 | TIC807_178 | E155W | none | None | ND | — | 5 | ND | — | ND |
| 178 | TIC807_178 | E155S | none | None | ND | — | 4 | ND | — | ND |
| 178 | TIC807_178 | E155V | none | None | ND | — | 4 | ND | — | ND |
| 179 | TIC807_179 | N157C | none | None | ND | — | 86 | ND | — | ND |
| 179 | TIC807_179 | N157D | none | None | ND | — | 64 | ND | — | ND |
| 179 | TIC807_179 | N157W | none | None | ND | — | 52 | ND | — | ND |
| 179 | TIC807_179 | N157Y | none | None | ND | — | 39 | ND | — | ND |
| 179 | TIC807_179 | N157M | none | None | ND | — | 22 | ND | — | ND |
| 179 | TIC807_179 | N157A | none | None | ND | — | 22 | ND | — | ND |
| 179 | TIC807_179 | N157F | none | None | ND | — | 20 | ND | — | ND |
| 179 | TIC807_179 | N157V | none | None | ND | — | 18 | ND | — | ND |
| 179 | TIC807_179 | N157L | none | None | ND | — | 18 | ND | — | ND |
| 179 | TIC807_179 | N157P | none | None | ND | — | 18 | ND | — | ND |
| 179 | TIC807_179 | N157E | none | None | ND | — | 17 | ND | — | ND |
| 179 | TIC807_179 | N157T | none | None | ND | — | 8 | ND | — | ND |
| 179 | TIC807_179 | N157G | none | None | ND | — | 7 | ND | — | ND |
| 179 | TIC807_179 | N157I | none | None | ND | — | 7 | ND | — | ND |
| 179 | TIC807_179 | N157R | none | None | ND | — | 6 | ND | — | ND |
| 202 | TIC807_M8_d5C | F46S, Y54H, S95A, | S95A, F147A | S217N, then a | ND | — | 92* | ND | — | ND |
| | | F147A, S167R, | | contiguous triple | | | | | | |
| | | S217N, then a five | | deletion in residue | | | | | | |
| | | amino acid deletion | | range 196-201 | | | | | | |
| | | at 305-309, then | | | | | | | | |
| | | a contiguous triple | | | | | | | | |
| | | deletion in residue | | | | | | | | |
| | | range 196-201 | | | | | | | | |
| 204 | TIC807_M14 | F46S, Y54H, S95A, | S95A, F147A, | P219R, then a | ND | — | — | ND | — | 60* |
| | | F147S, Q149E, S167R, | Q149E, V251E, | contiguous triple | | | | | | |
| | | P219R, S249R, V251E, | Q253R | deletion in residue | | | | | | |
| | | Q253R, R273W, then a | | range 196-201 | | | | | | |
| | | contiguous triple | | | | | | | | |
| | | deletion in residue | | | | | | | | |
| | | range 196-201 | | | | | | | | |

ND = Not Determined; *tested at about 5 μg/mL.

### Example 5: Insect Inhibitory Activities of Protein Members of the Present Invention

Proteins of the present invention, such as but not limited to TIC807 Ml (SEQ ID NO:6), TIC807 M2 (SEQ ID NO:8), TIC807 M3 (SEQ ID NO:10), TIC807 M4 (SEQ ID NO:12), TIC807 M5 (SEQ ID NO: 14), TIC807 M6 (SEQ ID NO:16), TIC807 M7 (SEQ ID NO:18), TIC807 M8 (SEQ ID NO:20), TIC807 M9 (SEQ ID NO:22), TIC807 M14 (SEQ ID NO:32), TIC807 M15 (SEQ ID NO:34), and TIC807 M16 (SEQ ID NO:36), are prepared and tested for bioactivity against pests of plants other than from Lygus.

Proteins TIC807 M10 (SEQ ID NO:24), TIC807 MII (SEQ ID NO:26), TIC807 M12 (SEQ ID NO: 28), and TIC807 M13 (SEQ ID NO:30) were prepared and tested for bioactivity against pests from the order Lepidoptera, Coleoptera, Heteroptera, and Homoptera. Protein TIC807 M5 (SEQ ID NO:14) was prepared and tested for bioactivity against Coleopteran pests. Bioassays were conducted to evaluate the effects of these proteins on insects as shown in Table 5. Feeding assays were conducted on an artificial diet containing the insecticidal protein. The insecticidal protein was prepared as described in example 3 and topically applied using an insect-specific artificial diet, depending on the insect being tested. The toxin was suspended in a buffer and applied at a rate of 500 μg/mL of sample per well, and in the case of TIC807 M5 of 1000 μg/mL, and then allowed to dry. Mean stunting scores and population mortalities were determined on three populations of 8 insects per insect species tested. Results were expressed as positive (+) for insect reactions such as stunting and mortality that were statistically significant compared to the untreated control. Results were expressed as negative (−) if the insects were similar to the UTC, that is, feeding diet to which the above buffer only has been applied.

TABLE 5
eHTP's demonstrate additional insect inhibitory activities against pests other than Lygus spp.

| Protein | μg/mL | CPB | WCR | ECB | SWCB | CEW | FAW | SGSB | NBSB | GPA |
|---|---|---|---|---|---|---|---|---|---|---|
| UTC | 0 | − | − | − | − | − | − | − | − | − |
| TIC807 M5 | 1000 | + | − | ND | ND | ND | ND | ND | ND | ND |
| TIC807 M10 | 500 | − | − | − | − | − | − | − | − | − |
| TIC807 M11 | 500 | + | − | − | − | − | − | − | − | − |
| TIC807 M12 | 500 | − | − | − | − | − | − | − | − | − |
| TIC807 M13 | 500 | + | − | − | − | − | − | − | − | − |

UTC = UnTreated Control;
ND = Not Determined
CPB = Colorado potato beetle (Leptinotarsa decemlineata);
WCR = western corn rootworm (Diabrotica virgifera);
ECB = European corn borer (Ostrinia nubilalis); southwestern corn borer (Diatraea grandiosella);
CEW = corn earworm (Helicoyerpo zea);
FAW = Fall armyworm (Spodoptera frugiperda);
SGSB = southern green stink bug (Nezara virudula);
NBSB = neotropical brown stink bug (Euschistus heros);
GPA = Green peach aphid (Myzus persicae).

The proteins of the present invention are also tested for bioactivity against a pest from the phylum Nematoda.

### Example 6: Plants Expressing Proteins of the Present Invention Exhibit Insect Inhibitory Activity

This example illustrates expression of proteins of the present invention in plants, and demonstrates that cotton plants expressing proteins of the present invention exhibit insect inhibitory activity.

Polynucleotide segments for use in expression of the proteins of the present invention in plants are made according to the methods set forth in U.S. Pat. No. 7,741,118. For example, toxin proteins having the amino acid sequence as set forth in SEQ ID NO:4 (TIC807 4), SEQ ID NO:6(TIC807 M1), SEQ ID NO:8 (TIC807 M2), SEQ ID NO: 10 (TIC807 M3), SEQ ID NO: 12 (TIC807 M4), SEQ ID NO:14 (TIC807 M5), SEQ ID NO:16 (TIC807 M8), SEQ ID NO:18 (TIC807 M6), SEQ ID NO:20 (TIC807 M7), SEQ ID NO:22 (TIC807 22), SEQ ID NO:24 (TIC807 24), SEQ ID NO: 26 (TIC807 26), SEQ ID NO:28 (TIC807 M9), SEQ ID NO:30 (TIC807 M10), SEQ ID NO:32 (TIC807 M11), and SEQ ID NO:34 (TIC807 M13), are expressed from polynucleotide segments designed for use in plants and encoding the proteins of the present invention, including the polynucleotide sequences as set forth in SEQ ID NO:186, SEQ ID NO:187, SEQ ID NO: 188, SEQ ID NO: 189, SEQ ID NO:190, SEQ ID NO:191, SEQ ID NO:192, SEQ ID NO:193, SEQ ID NO: 194, SEQ ID NO:195, SEQ ID NO: 196, SEQ ID NO:197, SEQ ID NO: 198, SEQ ID NO: 199, SEQ ID NO:200, and SEQ ID NO:201, respectively.

It is intended that polynucleotide segments (or polynucleotide molecules) encoding each of the variant proteins or insect inhibitory fragments thereof, be used alone or in combination with each other, or in combination with other insect inhibitory proteins or insect inhibitory agents such as dsRNA mediated gene suppression molecules. Such combinations designed to work in synergistic or compatible mechanism with the proteins of the present invention. The intention of these combinations is to achieve plants and plant cells protected from pest, particularly insect pest, infestation. The specific variant proteins within the scope of the invention include the proteins corresponding to SEQ ID NOs listed in Table 4B and described throughout the application as filed.

Polynucleotide segments from SEQ ID NO:188 (encodes for TIC807 M2, SEQ ID NO:8) and from SEQ ID NO:192 (encodes for TIC807 M8, SEQ ID NO:16) were each recombinantly engineered into expression constructs for cotton transformation.

Transgenic cotton plants (recombinant cotton plants) were produced and tested for efficacy. Regenerated (RO) transgenic plants were selected that were low in copy number and high in expression of the respective variant protein, as determined by various quantitative and semi-quantitative methods, e.g. PCR, ELISAs and Westerns. Expression levels in RO cotton leaf tissue typically ranged from 0.5 to 500 ppm fresh weight. RO plants expressing high levels of protein were transferred to soil and selfed. Thirty seed from each of the selfed RO plants were planted and progeny homozygous for the transgene were grown to flowering. Eleven to 18 plants per 4 to 5 events per each construct of this example were tested for efficacy against Lygus (Tables 6A, 6B, and 6C). The untransformed cotton cultivar, plants from the pooled negative segregate population (progeny not containing the transgene), and plants expressing TIC807 parent protein served as negative controls. A branch of a flowering stage cotton plant was enclosed in a mesh bag made from breathable plastic ‘pollination’ sleeves (Vilutis and Co. Inc., Frankfort, IL), and multiple branches set up in similar fashion. Each mesh bag was secured at the stem using a twist tie. About 4-6 Lygus hesperus nymphs (<24 hours post-hatch) were placed into a 1.4 ml conical tube (Matrix Technologies Corp., NH). The branch inside a mesh bag was infested with nymphs by sliding the uncapped conical tube into the mesh bag. Insects were allowed to feed for a period of 10-11 days before all surviving insects in the mesh bag were collected on dry ice. Survivors were weighed to obtain a gross mass. Percent mortality and mean survivor mass were calculated. Missing insects were included in the mortality percent mortality calculation. As shown in Tables 6A, 6B, and 6C, cotton plants expressing the variant proteins TIC807 M2 and TIC807 M8 significantly impacted the growth and development of Lygus hesperus nymphs. Based on these results, these plants, seed, expression constructs were advanced for further development.

TABLE 6A
Mean % mortality determined from flowering stage Lygus feeding assays with cotton plants
expressing the variant TIC807 proteins TIC807 M2 and TIC807 M8.

| | | | Mean % | | | | | Mortality |
|---|---|---|---|---|---|---|---|---|
| Plant Event ID | Protein | N | Mortality | Std Dev | SEM | Lo 95% | Up 95% | t group |
| 64 | TIC807 M8 | 18 | 78.889 | 19.967 | 4.706 | 68.959 | 88.818 | A |
| 49 | TIC807 M8 | 18 | 75.556 | 22.288 | 5.253 | 64.472 | 86.639 | A |
| 91 | TIC807 M2 | 18 | 74.444 | 20.356 | 4.798 | 64.321 | 84.567 | A |
| 20 | TIC807 M8 | 18 | 73.333 | 19.403 | 4.573 | 63.685 | 82.982 | A |
| 15 | TIC807 M8 | 18 | 66.667 | 25.668 | 6.050 | 53.902 | 79.431 | AB |
| | | | 625.668 | | | | | |
| 58 | TIC807 M2 | 18 | 65.556 | 19.166 | 4.517 | 56.025 | 75.086 | AB |
| 48 | TIC807 M2 | 18 | 64.444 | 21.206 | 4.998 | 53.899 | 74.990 | AB |
| 19 | TIC807 M2 | 18 | 53.333 | 25.668 | 6.050 | 40.569 | 66.098 | BC |
| 68 | TIC807 M2 | 18 | 47.778 | 25.795 | 6.080 | 34.950 | 60.605 | C |
| Negative | | 24 | 41.667 | 22.001 | 4.491 | 32.376 | 50.957 | C |

TABLE 6B
Mean Instar determined from flowering stage Lygus feeding assays with cotton plants
expressing the variant TIC807 proteins TIC807 M2 and TIC807 M8.

| | | | Mean | | | | | Instar |
|---|---|---|---|---|---|---|---|---|
| Plant Event ID | Construct | N | Instar | Std Dev | SEM | Lo 95% | Up 95% | t group |
| 64 | TIC807 M8 | 11 | 3.636 | 0.552 | 0.166 | 3.266 | 4.007 | C |
| 68 | TIC807 M2 | 16 | 3.949 | 0.803 | 0.201 | 3.521 | 4.377 | BC |
| 48 | TIC807 M2 | 16 | 4.042 | 0.604 | 0.151 | 3.720 | 4.364 | BC |
| 58 | TIC807 M2 | 17 | 4.069 | 0.802 | 0.194 | 3.657 | 4.481 | BC |
| 15 | TIC807 M8 | 15 | 4.094 | 0.747 | 0.193 | 3.681 | 4.508 | BC |
| 19 | TIC807 M2 | 17 | 4.100 | 0.698 | 0.169 | 3.741 | 4.459 | BC |
| 91 | TIC807 M2 | 12 | 4.125 | 0.829 | 0.239 | 3.598 | 4.652 | ABC |
| 49 | TIC807 M8 | 12 | 4.139 | 0.762 | 0.220 | 3.655 | 4.623 | ABC |
| 20 | TIC807 M8 | 14 | 4.298 | 0.918 | 0.245 | 3.768 | 4.828 | AB |
| Negative | | 24 | 4.599 | 0.774 | 0.158 | 4.273 | 4.926 | A |

TABLE 6C
Mean Survival Mass determined from flowering stage Lygus feeding assays with
cotton plants expressing the variant TIC807 proteins TIC807 M2 and TIC807 M8.

| | | | Mean | | | | | Survivor |
|---|---|---|---|---|---|---|---|---|
| | | | Survival | | | | | Mass |
| Plant Event ID | Construct | N | Mass | Std Dev | SEM | Lo 95% | Up 95% | t group |
| 64 | TIC807 M8 | 11 | 2.315 | 1.489 | 0.449 | 1.314 | 3.315 | C |
| 68 | TIC807 M2 | 16 | 3.548 | 1.325 | 0.331 | 2.843 | 4.254 | B |
| 58 | TIC807 M2 | 17 | 3.561 | 1.348 | 0.327 | 2.868 | 4.255 | B |
| 48 | TIC807 M2 | 16 | 3.596 | 1.436 | 0.359 | 2.831 | 4.362 | B |
| 91 | TIC807 M2 | 12 | 3.775 | 1.775 | 0.512 | 2.647 | 4.902 | AB |
| 49 | TIC807 M8 | 12 | 3.837 | 2.135 | 0.616 | 2.481 | 5.193 | AB |
| 19 | TIC807 M2 | 17 | 3.908 | 1.467 | 0.356 | 3.154 | 4.662 | AB |
| 20 | TIC807 M8 | 14 | 3.918 | 1.950 | 0.521 | 2.792 | 5.044 | AB |
| 15 | TIC807 M8 | 15 | 3.937 | 1.906 | 0.492 | 2.881 | 4.993 | AB |
| Negative | | 24 | 4.735 | 1.179 | 0.241 | 4.237 | 5.233 | A |

Std Dev = standard deviation
SEM = Standard error on the mean
Lo 95% = Lower limit at 95% confidence interval
Up 95% = Upper limit at 95% confidence interval
T grouping = Using a least significant difference test,
F value = 101.1756,
df = 15, 44,
Pr < 0.0001

In another example, cotton plants from five transgenic events expressing TIC807 Mll were tested in a field trial having natural Lygus infestation pressures. These plants demonstrated field efficacy compared to the non-transgenic recipient line (DP393 germplasm used for transformation). The average number of Lygus lineolaris insects on five plants per event was significantly lower than the average number of Lygus lineolaris insects on plants from the non-transgenic control. Seed cotton yield from plants from the five events was statistically comparable to seed cotton yield of the non-transgenic control, e.g. season-long square retention.

In another similar field trial, cotton plants from seven transgenic events expressing TIC807 M10 demonstrated field efficacy compared to the non-transgenic control. The average number of Lygus lineolaris insects on five plants per event was significantly lower than the average number of Lygus lineolaris insects on plants from the non-transgenic control. Seed cotton yield from plants from three of the seven events was statistically higher than to seed cotton yield of the non-transgenic control.

In another example, cotton plants from thirty-four transgenic events expressing TIC807 M13 demonstrated growth chamber efficacy compared to the non-transgenic control. Mesh bags were placed around the whole cotton plants at flowering stage (instead of just around single branches described earlier in this example). Five plants per event were evaluated and the average number of Lygus lineolaris insects recovered (nymphs to adults to 2^{nd} generation Lygus) per plant was significantly lower than the average number of Lygus lineolaris insects per non-transgenic plant.

Similar experiments are performed with plants expressing proteins listed in Table 1 and in Tables 4A and 4B.

### Example 7: Tissue From Alfalfa Plants Expressing Proteins of the Present Invention Exhibit Insect Inhibitory Activity

This example illustrates expression of proteins of the present invention in alfalfa plants, and demonstrates that tissue from alfalfa plants expressing proteins of the present invention exhibit insect inhibitory activity.

Polynucleotide segment from SEQ ID NO:192 (encodes for TIC807 M8, SEQ ID NO:16) was recombinantly engineered into three differently configured expression constructs for alfalfa transformation. For purposes of data reporting, the three recombinant constructs are coded [ER], [ES], and [ET].

Transgenic alfalfa plants (recombinant alfalfa plants) were recovered from transformants that were outcrossed and then selfed. Recombinant alfalfa plants were selected that were low in copy number and high in TIC807 expression as determined by RT-PCR and semi-quantitative Western methods, respectively. Alfalfa plant tissue from ten separate events were pooled, lyophilized, ground, and resuspended in stock buffer, 25 mM NaCarb, pH10.5. Plant tissue from Alfalfa having no TIC807 M8 expressing transgene was prepared for use as control. Stock preparations were serially diluted 100, 300, and 900 fold for incorporation into Lygus diet. Using the feeding assay method of Example 4, mortality and stunting scores were determined on day 5 and compared to controls (See Tables 7A and 7B; data were analyzed using JMP4 statistical software). For each test sample and each dilution, three populations of eight nymphs were subjected to this bioassay. Stunting scores correspond to visual mass ratings where 0 =no difference to negative control, 1=about 25% less mass, 2=about 50% less mass, and 3=about 75% less mass. The average of the stunting scores for each population of eight nymphs is reported.

TABLE 7A
Mean % mortality determined from Lygus feeding assays with diet incorporated with
tissue from alfalfa plants expressing the variant TIC807 protein TIC807 M8.

| | | | Mean | | | | | |
|---|---|---|---|---|---|---|---|---|
| | Sample | Dilution | Stunting | | | Lower | Upper | t |
| Construct | Source | fold | Score | Std Dev | SEM | 95% | 95% | Grouping |
| [ER] | Pooled Alfalfa | 100 | 2.00 | 0.00 | 0.00 | 2.00 | 2.00 | CD |
| TIC807 M8 | tissue from 10 | 300 | 0.00 | 0.00 | 0.00 | 0.00 | 0.00 | E |
| | events per | 900 | 0.00 | 0.00 | 0.00 | 0.00 | 0.00 | E |
| [ES] | construct | 100 | 3.00 | 0.00 | 0.00 | 3.00 | 3.00 | A |
| TIC807 M8 | | 300 | 2.33 | 0.58 | 0.33 | 0.90 | 3.77 | BC |
| | | 900 | 0.00 | 0.00 | 0.00 | 0.00 | 0.00 | E |
| [ET] | | 100 | 2.67 | 0.58 | 0.33 | 1.23 | 4.10 | AB |
| TIC807 M8 | | 300 | 1.67 | 0.58 | 0.33 | 0.23 | 3.10 | D |
| | | 900 | 0.00 | 0.00 | 0.00 | 0.00 | 0.00 | E |
| None | Control Alfalfa | 100 | 2.00 | 0.00 | 0.00 | 2.00 | 2.00 | CD |
| | | 300 | 0.00 | 0.00 | 0.00 | 0.00 | 0.00 | E |
| | | 900 | 0.00 | 0.00 | 0.00 | 0.00 | 0.00 | E |

| | | | | | | | |
|---|---|---|---|---|---|---|---|
| No Alfalfa incorporated | 0 | 0.00 | 0.00 | 0.00 | 0.00 | 0.00 | E |
| in the diet | | | | | | | |

TABLE 7B
Mean stunting determined from Lygus feeding assays with diet incorporated with tissue from
alfalfa plants expressing the variant TIC807 protein TIC807 M8.

| | | | Mean | | | | | |
|---|---|---|---|---|---|---|---|---|
| | | Dilution | Percent | | | Lower | Upper | t |
| Construct | Sample Source | fold | mortality | Std Dev | SEM | 95% | 95% | Grouping |
| [ER] | Pooled Alfalfa | 100 | 4.17 | 7.22 | 4.17 | −13.76 | 22.09 | CD |
| TIC807 M8 | tissue from 10 | 300 | 0.00 | 0.00 | 0.00 | 0.00 | 0.00 | CD |
| | events per | 900 | 13.10 | 12.54 | 7.24 | −18.06 | 44.25 | CD |
| [ES] | construct | 100 | 56.55 | 6.27 | 3.62 | 40.97 | 72.12 | AB |
| TIC807 M8 | | 300 | 41.67 | 19.09 | 11.02 | −5.77 | 89.10 | B |
| | | 900 | 0.00 | 0.00 | 0.00 | 0.00 | 0.00 | CD |
| [ET] | | 100 | 64.88 | 19.91 | 11.50 | 15.42 | 114.34 | A |
| TIC807 M8 | | 300 | 16.67 | 19.09 | 11.02 | −30.77 | 64.10 | C |
| | | 900 | 12.50 | 12.50 | 7.22 | −18.55 | 43.55 | CD |
| None | Control Alfalfa | | 12.50 | 12.50 | 7.22 | −18.55 | 43.55 | CD |
| | | | 0.00 | 0.00 | 0.00 | 0.00 | 0.00 | CD |
| | | | 8.33 | 14.43 | 8.33 | −27.52 | 44.19 | CD |

| | | | | | | | |
|---|---|---|---|---|---|---|---|
| No Alfalfa incorporated | 0 | 2.50 | 7.01 | 1.81 | −1.38 | 6.38 | D |
| in the diet | | | | | | | |

### Example 8: Plants Co-Expressing an eHTP and a Second Insect Inhibitory Protein Exhibiting Lygus Species Inhibitory Activity

Protein samples were prepared containing various mixtures of TIC1415 and TIC807_M13 and tested in bioassay. The TIC1415 protein and other Lygus inhibitory proteins are described in PCT Patent Application Publication No. WO 2012/139004. Sample mixtures were fed to Lygus lineolaris using bioactivity assay. TIC1415 protein alone and TIC807_M13 alone were also prepared as positive controls. Buffer was used as negative control. Samples from all three types of preparations exhibited mortality against Lygus lineolaris and survivors were stunted. Mortality and stunting scores were significant compared to bioactivity scores of insects fed with buffer (see Table 8A). The data suggests that there are no antagonistic effects. Additional bioassay tests are performed on mixtures to demonstrate synergistic and/or additive effects.

TABLE 8A
Bioassay data for protein mix: TIC1415 combined with TIC807_M13

| | | | Mean | | Mean | T Grouping |
|---|---|---|---|---|---|---|
| | TIC1415 | TIC807_M13 | Population | T Grouping | stuntingt | on |
| SAMPLE | (μg/mL) | (μg/mL) | mortality | on mort | score | stunting |
| TIC1415 + | 4.35 | 1 | 21.79 | AB* | 0.60 | AB* |
| TIC807_M13 | | | | | | |
| TIC1415 + | 2.175 | 1 | 20.36 | B* | 0.60 | AB* |
| TIC807_M13 | | | | | | |
| TIC1415 + | 1.0875 | 1 | 12.50 | BC | 0.60 | AB* |
| TIC807_M13 | | | | | | |
| TIC1415 + | 4.35 | 0.5 | 32.50 | A* | 0.80 | A* |
| TIC807_M13 | | | | | | |
| TIC1415 + | 1.75 | 0.265 | 7.86 | CD | 0.40 | ABC |
| TIC807_M13 | | | | | | |
| TIC1415 + | 0.875 | 0.265 | 0.00 | D | 0.00 | C |
| TIC807_M13 | | | | | | |
| TIC1415 + | 0.4375 | 0.265 | 5.36 | CD | 0.00 | C |
| TIC807_M13 | | | | | | |
| TIC1415 + | 4.35 | 0.25 | 13.21 | BC | 0.40 | ABC |
| TIC807_M13 | | | | | | |
| TIC1415 + | 1.75 | 0.1325 | 0.00 | D | 0.00 | C |
| TIC807_M13 | | | | | | |
| TIC1415 + | 1.75 | 0.06625 | 0.00 | D | 0.00 | C |
| TIC807_M13 | | | | | | |
| TIC1415 | 4.35 | 0 | 12.50 | BC | 0.40 | ABC |
| TIC1415 | 1.75 | 0 | 7.86 | CD | 0.00 | C |
| TIC807_M13 | 0 | 1 | 0.00 | D | 0.20 | BC |
| TIC807_M13 | 0 | 0.265 | 2.50 | CD | 0.00 | C |
| Buffer | 0 | 0 | 0.00 | D | 0.00 | C |
| (negative) | | | | | | |

Average (mean) of 5 populations of 8 nymphs per population.
Stunting scores correspond to visual mass ratings where 0 = no difference to negative control, 1 = about 25% less mass, 2 = about 50% less mass, and 3 = about 75% less mass. The average of the stunting scores for each population of eight nymphs is reported.
*At 95% confidence interval.

Cotton plants comprising events with transgenic DNA were designed to co-express respective proteins TIC1415 and TIC807_M13. Such plants were evaluated in a caged whole plant assay infested with Lygus lineolaris. Five plants each from ten events were caged and infested with 2 pairs of male and female L. lineolaris per plant. The assay was incubated in a growth chamber under normal environmental conditions for cotton plant development for 21 days. DP393 negative control plants were grown in similar manner. At the end of the 3 week period, Lygus of various stages of development were counted. The mean number per plant of Lygus hesperus insects at each stage in development were calculated (see Table 8B).

TABLE 8B
In-planta data for for protein mix: TIC1415 combined with TIC807_M13

| | | | | | | Mean | Mean | | |
|---|---|---|---|---|---|---|---|---|---|
| | | | Mean | Mean | Mean | Live | Total | | |
| | | | 3rd | 4th | 5th | 2nd | 2nd | | |
| | | | Instar | Instar | Instar | Gen. | Gen. | | Tukey |
| Construct | Event | N | or < | Nymph | Nymph | Adults | Lygus | SEM | Grouping |
| 12 | 021 | 5 | 0.00 | 0.00 | 0.00 | 0.00 | 0.00 | 0.00 | B |
| | 625 | 5 | 0.20 | 0.20 | 0.20 | 0.00 | 0.60 | 0.24 | B |
| | 830 | 5 | 2.20 | 0.20 | 0.00 | 0.00 | 2.40 | 1.12 | AB |
| | 890 | 5 | 4.40 | 0.00 | 0.20 | 0.00 | 4.60 | 2.62 | AB |
| | 521 | 5 | 4.60 | 0.60 | 0.00 | 0.00 | 5.20 | 4.27 | AB |
| | 980 | 5 | 3.40 | 1.20 | 1.20 | 0.00 | 5.80 | 4.86 | AB |
| 13 | 426 | 5 | 0.00 | 0.00 | 0.00 | 0.00 | 0.00 | 0.00 | B |
| | 611 | 5 | 0.60 | 0.00 | 0.00 | 0.00 | 0.60 | 0.60 | B |
| | 999 | 5 | 0.40 | 0.00 | 0.40 | 0.00 | 0.80 | 0.37 | B |
| | 356 | 5 | 6.20 | 0.00 | 0.40 | 0.00 | 6.60 | 4.73 | AB |
| | DP393 | | | | | | | | |
| Inbred | (Negative) | 10 | 7.00 | 2.50 | 0.80 | 0.00 | 10.30 | 3.75 | A |

What is claimed is:

1. An eHTP engineered from a scaffold toxin protein, wherein said eHTP exhibits Hemipteran insect species inhibitory activity against a Hemipteran pest species that is from about 2 to about 260 times greater than the Hemipteran inhibitory activity of said scaffold toxin; and optionally wherein said eHTP exhibits at least about 95% amino acid sequence identity to said toxin protein.

2. The eHTP of claim 1, said eHTP having the amino acid sequence as set forth in any of the proteins selected from the group consisting of SEQ ID NO:34, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO:28, SEQ ID NO:30, SEQ ID NO:32, SEQ ID NO:36, SEQ ID NO:37, SEQ ID NO:38, SEQ ID NO:39, SEQ ID NO:40, SEQ ID NO:41, SEQ ID NO:42, SEQ ID NO: 43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO: 54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO: 65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO: 87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO: 98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO: 109, SEQ ID NO:110, SEQ ID NO:111, SEQ ID NO:112, SEQ ID NO:113, SEQ ID NO: 114, SEQ ID NO:115, SEQ ID NO:116, SEQ ID NO:117, SEQ ID NO:118, SEQ ID NO: 119, SEQ ID NO:120, SEQ ID NO:121, SEQ ID NO:122, SEQ ID NO:123, SEQ ID NO: 124, SEQ ID NO:125, SEQ ID NO:126, SEQ ID NO:127, SEQ ID NO:128, SEQ ID NO: 129, SEQ ID NO:130, SEQ ID NO:131, SEQ ID NO:132, SEQ ID NO:133, SEQ ID NO: 134, SEQ ID NO:135, SEQ ID NO:136, SEQ ID NO:137, SEQ ID NO:138, SEQ ID NO: 139, SEQ ID NO:140, SEQ ID NO:141, SEQ ID NO:142, SEQ ID NO:143, SEQ ID NO: 144, SEQ ID NO:145, SEQ ID NO:146, SEQ ID NO:147, SEQ ID NO:148, SEQ ID NO: 149, SEQ ID NO:150, SEQ ID NO:151, SEQ ID NO:152, SEQ ID NO:153, SEQ ID NO: 154, SEQ ID NO:155, SEQ ID NO:156, SEQ ID NO:157, SEQ ID NO:158, SEQ ID NO: 159, SEQ ID NO:160, SEQ ID NO:161, SEQ ID NO:162, SEQ ID NO:163, SEQ ID NO: 164, SEQ ID NO:165, SEQ ID NO:166, SEQ ID NO:167, SEQ ID NO:168, SEQ ID NO: 169, SEQ ID NO:170, SEQ ID NO:171, SEQ ID NO:172, SEQ ID NO:173, SEQ ID NO: 174, SEQ ID NO:175, SEQ ID NO:176, SEQ ID NO:177, SEQ ID NO:178, SEQ ID NO: 179, SEQ ID NO:202, and SEQ ID NO:204, or an insect inhibitory fragment thereof.

3. The eHTP of claim 1, wherein said scaffold toxin protein is selected from the group consisting of SEQ ID NO:2 (TIC807), SEQ ID NO:8 (TIC807_M2), SEQ ID NO:182 (Cry51Aa1), SEQ ID NO: 184 (TIC853), and SEQ ID NO:206 (AXMI-171).

4. The eHTP of claim 1, further comprising at least one amino acid substitution, at least one amino acid addition, or at least one amino acid deletion in said scaffold toxin protein selected from the group consisting of SEQ ID NO:2 (TIC807), SEQ ID NO:8 (TIC807_M2), SEQ ID NO:182 (Cry51Aa1), SEQ ID NO:184 (TIC853), and SEQ ID NO:206 (AXMI-171).

5. The eHTP of claim 4, wherein in the alternative:
(i) said amino acid substitution, addition, or deletion is in a solvent accessible amino acid residue of SEQ ID NO:2;
(ii) said amino acid substitution, addition, or deletion is within 3 consecutive amino acid residues of a solvent accessible amino acid residue of SEQ ID NO:2; or
(iii) said eHTP has the amino acid sequence as set forth in SEQ ID NO:180.

6. The eHTP of claim 5, wherein said eHTP comprises at least one substitution or deletion selected from the group consisting of asparagine at position 12 replaced by aspartic acid, phenylalanine at position 46 replaced by serine, isoleucine at position 52 replaced by methionine, tyrosine at position 54 replaced by histidine, threonine at position 68 replaced by alanine, glutamine at position 70 replaced by alanine, alanine at position 87 replaced by serine, threonine at position 93 replaced by alanine, serine at position 95 replaced by alanine, glycines at position 105 replaced by alanine, serine at position 117 replaced by alanine, serine at position 119 replaced by alanine, glutamate at position 125 replaced by cysteine, histidine, arginine, phenylalanine, serine, glutamine, lysine, threonine, asparagine, alanine, leucine, valine, methionine, aspartic acid, or tyrosine, glycines at position 128 replaced by alanine, threonine at position 133 replaced by glutamic acid, tyrosine, or tryptophan, isoleucine at position 134 replaced by alanine, valine, leucine, phenylalanine, lysine, cysteine, or methionine, glutamate at position 135 replaced by serine, alanine, valine, tryptophan, or threonine, asparagine at position 137 replaced by histidine, tyrosine, threonine, glutamic acid, serine, alanine, glutamine, glycine, isoleucine, tryptophan, lysine, cysteine, methionine, aspartic acid, phenylalanine, or arginine, phenylalanine at position 138 replaced by valine, Ala139 replaced by serine, Thr145 replaced by alanine, Phe 147 replaced by serine, valine, threonine, cysteine, leucine, aspartic acid, alanine, glycine, glutamic acid, isoleucine, tyrosine, methionine, asparagine, glutamine, hystidine, alanine, arginine, tryptophan, or proline, glutamine at position 148 replaced by alanine, glutamine at position 149 replaced by aspartic acid, glutamic acid, cysteine, alanine, or phenylalanine, alanine at position 150 replaced by serine, leucine, valine, glycine, aspartic acid, tryptophan, glutamic acid, asparagine, tyrosine, phenylalanine, proline, lysine, threonine, glutamine, or arginine, seroine at position 151 replaced by alanine, aspartate at position 153 replaced by alanine, glutamate at position 155 replaced by cysteine, isoleucine, lysine, aspartic acid, histidine, tyrosine, glutamine, lysine, asparagine, threonine, alanine, phenylalanine, arginine, methionine, proline, tryptophan, serine, or valine, asparagine at position 157 replaced by cysteine, aspartic acid, tryptophan, tyrosine, methionine, alanine, phenylalanine, valine, leucine, proline, glutamic acid, threonine, glycine, isoleucine, or arginine, isoleucine at position 158 replaced by alanine, serine at position 159 replaced by alanine or threonine, serine at position 167 replaced by arginine or alanine, valine at position 175 replaced by alanine, methionine at position 177 replaced by alanine, asparagine at position 180 replaced by aspartic acid, threonine at position 182 replaced by alanine, leucine at position 187 replaced by alanine, histidine at position 196 deleted, tyrosine at position 197 deleted, serine at position 198 deleted, histidine at position 199 deleted, tyrosine at position 200 replaced by alanine, tyrosine at position 200 deleted, Ser201 replaced by alanine, serine at position 201 deletion, tryptophan at position 208 replaced by alanine, serine at position 217 replaced by asparagine, proline at position 219 replaced by arginine, tryptophan at position 223 replaced by tyrosine, phenylalanine at position 235 replaced by alanine, asparagine at position 239 replaced by alanine, aspartate at position 241 replaced by alanine, threonine at position 243 replaced by alanine, valine at position 244 replaced by isoleucine, threonine at position 245 replaced by alanine, tyrosine at position 246 replaced by phenylalanine, threonine at position 247 replaced by alanine or lysine, serine at position 249 replaced by alanine or arginine, valine at position 250 replaced by alanine, valine at position 251 replaced by alanine, serine at position 252 replaced by alanine, arginine at position 273 replaced by tryptophan, threonine at position 274 replaced by alanine, isoleucine at position 275 replaced by alanine, arginine at position 282 replaced by alanine, histidine at position 287 replaced by alanine or phenylalanine, serine at position 293 replaced by alanine, asparagine at position 295 replaced by alanine, glutamate at position 299 replaced by alanine, methionine at position 300 replaced by alanine, threonine at position 303 replaced by alanine, proline at position 305 replaced by alanine, isoleucine at position 306 replaced by alanine, and threonine at position 308 replaced by alanine;
wherein said substitution or deletion is made with reference to the scaffold toxin protein TIC807 as set forth in SEQ ID NO:2, or to the TIC807 corresponding amino acid residue position of TIC807_M2 as set forth in SEQ ID NO:8, Cry51Aa1 as set forth in SEQ ID NO:182, or TIC853 as set forth in SEQ ID NO:184; and
wherein said eHTP comprises any combination of said substitutions or deletions.

7. The eHTP of claim 6, wherein said eHTP comprises at least one amino acid substitution, one amino acid addition, or one amino acid deletion at an amino acid residue of SEQ ID NO:2 selected from (i) an amino acid residue having a relative solvent-accessibility of at least from about 15% to about 36%; and (ii) an amino acid residue located within a distance of about 3 consecutive residues from an amino acid having at least from about 15% to about 36% relative solvent-accessibility.

8. The eHTP of claim 7, wherein said eHTP comprises optionally:
a. at least one amino acid substitution, one amino acid addition, or one amino acid deletion at an amino acid residue of SEQ ID NO:2 selected from the group consisting of Thr93, Ser95, Ser97, Phe147, Gln149, Ser151, Asn180, Thr182, Val251, Gln253, and Ser255;
b. at least one additional amino acid substitution, one amino acid addition, or one amino acid deletion at an amino acid residue of SEQ ID NO: 2 selected from the group consisting of Val10, Ile14, Asn22, Asn23, Gly24, Ile25, Gln26, Gly27, Phe30, Gln38, Ile39, Asp40, Thr41, Ile43, Ser193, Thr194, Glu195, His196, Tyr197, Ser198, His199, Tyr200, Ser201, Gly202, Tyr203, Pro204, Ile205, Leu206, Thr207, Trp208, Ile209, Ser210, Tyr216, Ser217, Gly218, Pro219, Pro220, Met221, Ser222, Trp223, Tyr224, Phe225, Asn239, and Val244;
c. at least one amino acid substitution, one amino acid addition, or one amino acid deletion at an amino acid residue of SEQ ID NO: 2 selected from the group consisting of Val10, Ile14, Asn22, Asn23, Gly24, Ile25, Gln26, Gly27, Phe30, Gln38, Ile39, Asp40, Thr41, Ile43, Ser193, Thr194, Glu195, His196, Tyr197, Ser198, His199, Tyr200, Ser201, Gly202, Tyr203, Pro204, Ile205, Leu206, Thr207, Trp208, Ile209, Ser210, Tyr216, Ser217, Gly218, Pro219, Pro220, Met221, Ser222, Trp223, Tyr224, Phe225, Asn239, and Val244;
d. one or more mutations in SEQ ID NO:2 selected from the group consisting of S95A, F147A, Q149E, V251A, P219R; or
e. a contiguous triple deletion in SEQ ID NO:2 in residue range 196-201.

9. The eHTP of claim 8, further modified to exhibit increased solubility compared to the native Bacillus thuringiensis protein as set forth in SEQ ID NO:2, wherein said eHTP comprises at least one modification relative to SEQ ID NO:2, said modification comprising a lysine substitution at one or more of the following amino acid positions: 58, 59, 198, 199, 201, or 202; or a glutamic acid substitution at one or more of the amino acid positions 198, 248, or 301; or an arginine substitution at one or more of the amino acid positions 246, 250, or 253.

10. The eHTP of claim 9, wherein said Hemipteran pest species is selected from the group consisting of Lygus hesperus, Lygus lineolaris, and Amrasca devastans.

11. An insect inhibitory composition comprising the eHTP of claim 10.

12. The insect inhibitory composition of claim 11, further comprising at least one insect inhibitory agent different from said eHTP, wherein said insect inhibitory agent is selected from the group consisting of an insect inhibitory protein, an insect inhibitory dsRNA molecule, and an insect inhibitory chemistry;
wherein said insect inhibitory protein different from said eHTP is selected from the group consisting of a Cry1 toxin, a Cry2 toxin, a Cry 5 toxin, a Cry7 toxin, a Cry9 toxin, and a VIP3 toxin; and
wherein said insect inhibitory chemistry is selected from the group consisting of pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates.

13. A method of controlling a Hemipteran pest, comprising contacting said pest with a Hemipteran inhibitory amount of the eHTP of claim 10, wherein said Hemipteran pest is in a cotton field.

14. A recombinant polynucleotide encoding the eHTP of claim 10, and optionally comprising:
a. the nucleotide sequence selected from the group consisting of SEQ ID NO:33, SEQ ID NO: 1, SEQ ID NO:3, SEQ ID NO:5, SEQ ID NO:7, SEQ ID NO:9, SEQ ID NO:11, SEQ ID NO: 13, SEQ ID NO:15, SEQ ID NO:17, SEQ ID NO:19, SEQ ID NO:21, SEQ ID NO: 23, SEQ ID NO:25, SEQ ID NO:27, SEQ ID NO:29, SEQ ID NO:31, SEQ ID NO:35, SEQ ID NO:186, SEQ ID NO:187, SEQ ID NO:188, SEQ ID NO:189, SEQ ID NO: 190, SEQ ID NO:191, SEQ ID NO:192, SEQ ID NO: 193, SEQ ID NO:194, SEQ ID NO:195, SEQ ID NO:196, SEQ ID NO:197, SEQ ID NO: 198, SEQ ID NO:199, SEQ ID NO:200, SEQ ID NO:201, and SEQ ID NO:203; or
b. a nucleotide sequence encoding one or more insect inhibitory agents that are different from the eHTP encoded by said recombinant polynucleotide.

15. A transgenic plant cell, plant or plant part comprising the recombinant polynucleotide of claim 14, and wherein optionally:
a. said plant part is a seed, a boll, a leaf, a flower, a stem, a root, or any portion thereof; or
b. said plant part is a non-regenerable portion of said seed, boll, leaf, flower, stem, or root.

16. A method of controlling a Hemipteran pest, comprising exposing the pest to the transgenic plant cell, plant or plant part of claim 15, wherein said plant cell, plant or plant part expresses a Hemipteran inhibitory amount of said eHTP.

17. A commodity product derived from the plant cell, plant or plant part of claim 15, wherein said product comprises a detectable amount of said recombinant polynucleotide; and wherein said product is selected from the group consisting of plant biomass, oil, meal, animal feed, flour, flakes, bran, lint, hulls, and processed seed; and wherein optionally said product is non-regenerable.

18. A method of making a plant resistant to Hemipteran pest infestation, comprising the steps of introducing a recombinant polynucleotide encoding an eHTP into a plant cell; regenerating from said plant cell a transgenic plant expressing an insect inhibitory amount of the eHTP; and demonstrating Hemipteran pest infestation resistance as a property of said transgenic plant; wherein said plant optionally:
a. is selected from the group consisting of a dicot plant and a monocot plant;
b. is selected from the group consisting of alfalfa, almont, banana, barley, bean, beet, broccoli, cabbage, brassica, brinjal, carrot, cassava, castor, cauliflower, celery, chickpea, Chinese cabbage, celery, citrus, coconut, coffee, corn, clover, cotton, a cucurbit, cucumber, Douglas fir, eggplant, eucalyptus, flax, garlic, grape, guar, hops, leek, legumes, lettuce, Loblolly pine, millets, melons, nectarine, nut, oat, okra, olive, onion, ornamental, palm, pasture grass, papaya, pea, peach, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat; or
c. comprises a supplemental agent selected from the group consisting of an insect inhibitory protein different from said eHTP, an insect inhibitory dsRNA molecule, and an insect inhibitory chemistry;
wherein said insect inhibitory protein different from said eHTP is selected from the group consisting of a Cry1 toxin, a Cry2 toxin, a Cry 5 toxin, a Cry7 toxin, a Cry9 toxin, and a VIP3 toxin; and
wherein said insect inhibitory chemistry is selected from the group consisting of pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates.

19. The method of claim 18, wherein said Hemipteran insect pest is selected from the group consisting of plant bugs (including the Family Miridae), cicadas from the Family Cicadidae, leafhoppers (e. g., Empoasca spp., Amrasca spp. from the Family Cicadellidae including the tribe Empoascini, e.g. Amrasca biguttula, Amrasca devastans, Austroasca viridigrisea, Asymmetrasca decedens, Empoasca decipiens, Empoasca distinguenda, Empoasca dolichi, Empoasca fabae, Empoasca kerri, Empoasca kraemeri, Empoasca onukii, Empoasca sakaii, Empoasca smithi, Empoasca vitis, Jacobiasca lybica, Sonasasca Solana, tribe Erythroneurini, e.g. Empoascanara nagpurensis, Thaiaassamensis, Zygnidia quyumi, tribe Nirvaniae, Sophonia rufofascia, Family Delphacidae, e.g. Nilapoarvata lugens, Sogatella furcifera, Unkanodes sapporonus, and Family Lophopidae, e.g. Zophiuma lobulata), planthoppers (from the families Fulgoroidea and Delphacidae), treehoppers (from the Family Membracidae), psyllids (from the Family Psyllidae), whiteflies (from the Family Aleyrodidae), aphids (from the Family Aphididae), phylloxera (from the Family Phylloxeridae), mealybugs (from the Family Pseudococcidae), scales (from the families Coccidae, Diaspididae and Margarodidae), lace bugs (from the Family Tingidae), stink bugs (from the Family Pentatomidae), cinch bugs (e. g., Blissus spp. and other seed bugs from the Family Lygaeidae), spittlebugs (from the Family Cercopidae), squash bugs (from the Family Coreidae), red bugs and cotton stainers (from the Family Pyrrhocoridae), Acrosternum hilare (green stink bug), Anasa tristis (squash bug), Blissus leucopterus leucopterus (chinch bug), Corythuca gossypii (cotton lace bug), Cyrtopeltis modesta (tomato bug), Dysdercus suturellus (cotton stainer), Euschistus servus (brown stink bug), Euschistus variolarius (one-spotted stink bug), Graptostethus spp. (complex of seed bugs), Leptoglossus corculus (leaf-footed pine seed bug), Lygus lineolaris (tarnished plant bug), Lygus hesperus (Western tarnish plant bug), Nezara viridula (southern green stink bug), Oebalus pugnax (rice stink bug), Oncopeltus fasciatus (large milkweed bug), and Pseudatomoscelis seriatus (cotton fleahopper).

20. An eHTP (engineered Hemipteran toxic protein) selected from the group consisting of SEQ ID NO: 34, of SEQ ID NO:6, SEQ ID NO:8, SEQ ID NO:10, SEQ ID NO: 12, SEQ ID NO:14, SEQ ID NO:16, SEQ ID NO:18, SEQ ID NO:20, SEQ ID NO:28, SEQ ID NO:30, SEQ ID NO: 32, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO: 57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO: 79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO: 90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO: 101, SEQ ID NO:102, SEQ ID NO:103, SEQ ID NO:104, SEQ ID NO:105, SEQ ID NO: 106, SEQ ID NO:107, SEQ ID NO:108, SEQ ID NO:109, SEQ ID NO:110, SEQ ID NO: 111, SEQ ID NO:112, SEQ ID NO:113, SEQ ID NO:114, SEQ ID NO:115, SEQ ID NO: 116, SEQ ID NO:117, SEQ ID NO:118, SEQ ID NO:119, SEQ ID NO:120, SEQ ID NO: 121, SEQ ID NO:122, SEQ ID NO:123, SEQ ID NO:124, SEQ ID NO:125, SEQ ID NO: 126, SEQ ID NO:127, SEQ ID NO:128, SEQ ID NO:129, SEQ ID NO:130, SEQ ID NO: 131, SEQ ID NO:132, SEQ ID NO:133, SEQ ID NO:134, SEQ ID NO:135, SEQ ID NO: 136, SEQ ID NO:137, SEQ ID NO:138, SEQ ID NO:139, SEQ ID NO:140, SEQ ID NO: 141, SEQ ID NO:142, SEQ ID NO:143, SEQ ID NO:144, SEQ ID NO:145, SEQ ID NO: 146, SEQ ID NO:147, SEQ ID NO:148, SEQ ID NO:149, SEQ ID NO:150, SEQ ID NO: 151, SEQ ID NO:152, SEQ ID NO:153, SEQ ID NO:154, SEQ ID NO:155, SEQ ID NO: 156, SEQ ID NO:157, SEQ ID NO:158, SEQ ID NO:159, SEQ ID NO:160, SEQ ID NO: 161, SEQ ID NO:162, SEQ ID NO:163, SEQ ID NO:164, SEQ ID NO:165, SEQ ID NO: 166, SEQ ID NO:167, SEQ ID NO:168, SEQ ID NO:169, SEQ ID NO:170, SEQ ID NO: 171, SEQ ID NO:172, SEQ ID NO:173, SEQ ID NO:174, SEQ ID NO:175, SEQ ID NO: 176, SEQ ID NO:177, SEQ ID NO:178, SEQ ID NO: 179, SEQ ID NO:202, and SEQ ID NO: 204, or an insect inhibitory fragment thereof.


SEQUENCE LISTING (206 sequences)

SEQ ID NO: 1 - DNA, 927 bp, Bacillus thuringiensis
  misc_feature 1..927: note = A sequence representing a recombinant polynucleotide derived from a native gene from a Bacillus thuringiensis (Bt) species encoding a TIC807 protein.
  source 1..927: mol_type = other DNA
atggctatcctagaccttaagtccctcgtgctgaacgccattaactactggggccctaagaacaacaacggcatccagggcggtgacttcggctaccccatctctgagaagcagatcgacactagcatcattaccttcacccaccctcgcttgatcccctacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtgctggttcctccctctaaacaagttgtcgcgacccttgtgatcatgggaggcaactttaccatccctatggacttgatgaccacgattgatagtacagagcactactcccactactccggttaccctatcctcacctggatctcgtccccagataactcttactccggtccctttatgtcatggtactttgcaaactggcctaaccttccgagtggattcggcccactgaatagtgataacacggtcacatacactggctctgtcgtgtcccaagtttcggccggtgtctacgctaccgtccggttcgatcagtatgacattcacaatctccgtactatcgagaagacttggtatgctcgccatgcgacgctgcataatggcaagaagatttctatcaacaatgtcacggaaatggctccaacatcccctatcaagacaaat

SEQ ID NO: 2 - protein, 309 aa, synthetic construct
  REGION 1..309: note = engineered
  REGION 1..309: note = TIC807
  REGION 1..309: note = The amino acid sequence translation of of the open reading frame of SEQ ID NO: 1 from nucleotides 1 through 927.
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 3 - DNA, 927 bp, synthetic construct
  misc_feature 1..927: note = engineered
  misc_feature 1..927: note = A nucleotide sequence encoding SEQ ID NO: 4 with an open reading frame from 1 to 927.
  source 1..927: mol_type = other DNA
atggctatcctagaccttaagtccctcgtgctgaacgccattaactactggggccctaagaacaacaacggcatccagggcggtgacttcggctaccccatctctgagaagcagatcgacactagcatcattaccttcacccaccctcgcttgatcccctacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgcgggtggcgggaagaactcgactaccatagaggccaacttcgcacacaactctagcaccactaccttccagcaagcaagcactgacattgagtggaacattagccaaccggtgctggttcctccctctaaacaagttgtcgcgacccttgtgatcatgggaggcaactttaccatccctatggacttgatgaccacgattgatagtacagagcactactcccactactccggttaccctatcctcacctggatctcgtccccagataactcttactccggtccctttatgtcatggtactttgcaaactggcctaaccttccgagtggattcggcccactgaatagtgataacacggtcacatacactggctctgtcgtgtcccaagtttcggccggtgtctacgctaccgtccggttcgatcagtatgacattcacaatctccgtactatcgagaagacttggtatgctcgccatgcgacgctgcataatggcaagaagatttctatcaacaatgtcacggaaatggctccaacatcccctatcaagacaaat

SEQ ID NO: 4 - protein, 309 aa, synthetic construct
  REGION 1..309: note = engineered
  REGION 1..309: note = The amino acid sequence translation of of the open reading frame set forth as SEQ ID NO: 3.
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGAGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 5 - DNA, 927 bp, synthetic construct
  misc_feature 1..927: note = engineered
  misc_feature 1..927: note = A nucleotide sequence encoding SEQ ID NO: 6 with an open reading frame from 1 to 927.
  source 1..927: mol_type = other DNA
atggctatcctagaccttaagtccctcgtgctgaacgccattaactactggggccctaagaacaacaacggcatccagggcggtgacttcggctaccccatctctgagaagcagatcgacactagcatcattaccttcacccaccctcgcttgatcccctacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtgctggttcctccctctaaacaagttgtcgcgacccttgtgatcatgggaggcaactttaccatccctatggacttgatgaccacgattgatagtacagagcactactcccactactccggttaccctatcctcacctggatctcgtccccagataactcttactccggtccctttatgtcatggtactttgcaaactggcctaaccttccgagtggattcggcccactgaatagtgataacacggtcacatacactggctctgtcgtgtcccaagtttcggccggtgtctacgctaccgtccggttcgatcagtatgacattcacaatctccgtactatcgagaagacttggtatgctcgccatgcgacgctgcataatggcaagaagatttctatcaacaatgtcacggaaatggctccaacatcccctatcaagacaaat

SEQ ID NO: 6 - protein, 309 aa, synthetic construct
  REGION 1..309: note = engineered
  REGION 1..309: note = The amino acid sequence translation of the open reading frame set forth as SEQ ID NO: 5.
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 7 - DNA, 918 bp, synthetic construct
  misc_feature 1..918: note = engineered
  misc_feature 1..918: note = A nucleotide sequence encoding SEQ ID NO: 8 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 8 - protein, 306 aa, synthetic construct
  REGION 1..306: note = engineered
  REGION 1..306: note = TIC807M2
  REGION 1..306: note = The amino acid sequence translation of the open reading frame set forth as SEQ ID NO: 7.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 9 - DNA, 918 bp, synthetic construct
  misc_feature 1..918: note = engineered
  misc_feature 1..918: note = A nucleotide sequence encoding SEQ ID NO: 10 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 10 - protein, 306 aa, synthetic construct
  REGION 1..306: note = engineered
  REGION 1..306: note = The amino acid sequence translation of the open reading frame set forth as SEQ ID NO: 9.
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 11 - DNA, 918 bp, synthetic construct
  misc_feature 1..918: note = engineered
  misc_feature 1..918: note = A nucleotide sequence encoding SEQ ID NO: 12 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 12 - protein, 306 aa, synthetic construct
  REGION 1..306: note = engineered
  REGION 1..306: note = The amino acid sequence translation of the open reading frame set forth as SEQ ID NO: 11.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 13 - DNA, 918 bp, synthetic construct
  misc_feature 1..918: note = engineered
  misc_feature 1..918: note = A nucleotide sequence encoding SEQ ID NO: 14 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 14 - protein, 306 aa, synthetic construct
  REGION 1..306: note = engineered
  REGION 1..306: note = The amino acid sequence translation of the open reading frame set forth as SEQ ID NO: 13.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 15 - DNA, 918 bp, synthetic construct
  misc_feature 1..918: note = engineered
  misc_feature 1..918: note = A nucleotide sequence encoding SEQ ID NO: 16 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 16 - protein, 306 aa, synthetic construct
  REGION 1..306: note = engineered
  REGION 1..306: note = TIC807M8
  REGION 1..306: note = The amino acid sequence translation of the open reading frame set forth as SEQ ID NO: 15.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 17 - DNA, 918 bp, synthetic construct
  misc_feature 1..918: note = engineered
  misc_feature 1..918: note = A nucleotide sequence encoding SEQ ID NO: 18 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 18 - protein, 306 aa, synthetic construct
  REGION 1..306: note = engineered
  REGION 1..306: note = The amino acid sequence translation of the open reading frame set forth as SEQ ID NO: 17.
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 19 - DNA, 918 bp, synthetic construct
  misc_feature 1..918: note = engineered
  misc_feature 1..918: note = A nucleotide sequence encoding SEQ ID NO: 20 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggaggcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 20 - protein, 306 aa, synthetic construct
  REGION 1..306: note = engineered
  REGION 1..306: note = The amino acid sequence translation of the open reading frame set forth as SEQ ID NO: 19.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQEASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 21 - DNA, 918 bp, synthetic construct
  misc_feature 1..918: note = engineered
  misc_feature 1..918: note = A nucleotide sequence encoding SEQ ID NO: 22 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggcagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 22 - protein, 306 aa, synthetic construct
  REGION 1..306: note = engineered
  REGION 1..306: note = The amino acid sequence translation of the open reading frame set forth as SEQ ID NO: 21.
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQAASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 23 - DNA, 918 bp, synthetic construct
  misc_feature 1..918: note = engineered
  misc_feature 1..918: note = A nucleotide sequence encoding SEQ ID NO: 24 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggaggcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 24 - protein, 306 aa, synthetic construct
  REGION 1..306: note = engineered
  REGION 1..306: note = The amino acid sequence translation of the open reading frame set forth as SEQ ID NO: 23.
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQEASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 25 - DNA, 918 bp, synthetic construct
  misc_feature 1..918: note = engineered
  misc_feature 1..918: note = A nucleotide sequence encoding SEQ ID NO: 26 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 26 - protein, 306 aa, synthetic construct
  REGION 1..306: note = engineered
  REGION 1..306: note = The amino acid sequence translation of the open reading frame set forth as SEQ ID NO: 25.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 27 - DNA, 918 bp, synthetic construct
  misc_feature 1..918: note = engineered
  misc_feature 1..918: note = A nucleotide sequence encoding SEQ ID NO: 28 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 28 - protein, 306 aa, synthetic construct
  REGION 1..306: note = engineered
  REGION 1..306: note = The amino acid sequence translation of the open reading frame set forth as SEQ ID NO: 27.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 29 - DNA, 918 bp, synthetic construct
  misc_feature 1..918: note = engineered
  misc_feature 1..918: note = A nucleotide sequence encoding SEQ ID NO: 30 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgccagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 30 - protein, 306 aa, synthetic construct
  REGION 1..306: note = engineered
  REGION 1..306: note = TIC807M10
  REGION 1..306: note = The amino acid sequence translation of the open reading frame set forth as SEQ ID NO: 29.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVASQVSAGVYATVRFDQYDIHNLRTIEKTWYARHATLHNGKKISINNVTEMAPTSPIKTN

SEQ ID NO: 31 - DNA, 918 bp, synthetic construct
  misc_feature 1..918: note = engineered
  misc_feature 1..918: note = A nucleotide sequence encoding SEQ ID NO: 32 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctctggactattgagaagacctggtacgctcgtcatgcgacgcttcacaacggcaagaagatcagcatcaataacgtgacagaaatggcccctaccagcccgatcaagactaac

SEQ ID NO: 32 - protein, 306 aa, synthetic construct
  REGION 1..306: note = engineered
  REGION 1..306: note = The amino acid sequence translation of the open reading frame set forth as SEQ ID NO: 31.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVVSQVSAGVYATVRFDQYDIHNLWTIEKTWYARHATLHNGKKISINNVTEMAPTSPIKTN

SEQ ID NO: 33 - DNA, 918 bp, synthetic construct
  misc_feature 1..918: note = engineered
  misc_feature 1..918: note = A nucleotide sequence encoding SEQ ID NO: 34 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactacctcccaggaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctctggactattgagaagacctggtacgctcgtcatgcgacgcttcacaacggcaagaagatcagcatcaataacgtgacagaaatggcccctaccagcccgatcaagactaac

SEQ ID NO: 34 - protein, 306 aa, synthetic construct
  REGION 1..306: note = engineered
  REGION 1..306: note = TIC807M13
  REGION 1..306: note = The amino acid sequence translation of the open reading frame set forth as SEQ ID NO: 33.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTSQEASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVVSQVSAGVYATVRFDQYDIHNLWTIEKTWYARHATLHNGKKISINNVTEMAPTSPIKTN

SEQ ID NO: 35 - DNA, 918 bp, synthetic construct
  misc_feature 1..918: note = engineered
  misc_feature 1..918: note = A nucleotide sequence encoding SEQ ID NO: 36 with an open reading frame from 1 to 918.
  source 1..918: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttgcctctgataacactgtgacctacactggctctgtcgccagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaac

SEQ ID NO: 36 - protein, 306 aa, synthetic construct
  REGION 1..306: note = engineered
  REGION 1..306: note = The amino acid sequence translation of the open reading frame set forth as SEQ ID NO: 35.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYSGRFMSWYFANWPNLPSGFGPLASDNTVTYTGSVASQVSAGVYATVRFDQYDIHNLRTIEKTWYARHATLHNGKKISINNVTEMAPTSPIKTN

SEQ ID NO: 37 - protein, 306 aa, synthetic construct
  REGION 1..306: note = engineered
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPSKQVVATLVIMGGNFTIPMDLMTTIDSTEHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 38 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANVAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 39 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLFNGKKISINNVTEMAPTSPIKTN

SEQ ID NO: 40 - protein, 306 aa, synthetic construct
  REGION 1..306: note = engineered
MAILDLKSLVLNAINYWGPKNNNGIQGGDFGYPISEKQIDTSIITSTHPRLMPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 41 - protein, 306 aa, synthetic construct
  REGION 1..306: note = engineered
MAILDLKSLVLDAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 42 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGDFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 43 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 44 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGRFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 45 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNITQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 46 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYKGSVVSQVSAGVYATVRFDQYDIHNLRTIEKTWYARHATLHNGKKISINNVTEMAPTSPIKTN

SEQ ID NO: 47 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTITYTGSVVSQVSAGVYATVRFDQYDIHNLRTIEKTWYARHATLHNGKKISINNVTEMAPTSPIKTN

SEQ ID NO: 48 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTITYKGSVVSQVSAGVYATVRFDQYDIHNLRTIEKTWYARHATLHNGKKISINNVTEMAPTSPIKTN

SEQ ID NO: 49 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSYYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 50 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTFTGSVVSQVSAGVYATVRFDQYDIHNLRTIEKTWYARHATLHNGKKISINNVTEMAPTSPIKTN

SEQ ID NO: 51 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGAKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 52 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEAAPTSPIKTN

SEQ ID NO: 53 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKIAINNVTEMAPTSPIKTN

SEQ ID NO: 54 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLANGKKISINNVTEMAPTSPIKTN

SEQ ID NO: 55 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRAIEKTWYARHATLHNGKKISINNVTEMAPTSPIKTN

SEQ ID NO: 56 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYAAHATLHNGKKISINNVTEMAPTSPIKTN

SEQ ID NO: 57 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKAN

SEQ ID NO: 58 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTAVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 59 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGAGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 60 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYAGSVVSQVSAGVYATVRFDQYDIHNLRTIEKTWYARHATLHNGKKISINNVTEMAPTSPIKTN

SEQ ID NO: 61 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSAIKTN

SEQ ID NO: 62 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPAKTN

SEQ ID NO: 63 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYAAHATLHNGKKISINNVTEMAPTSPIKTN

SEQ ID NO: 64 - protein, 306 aa, synthetic construct; identical to SEQ ID NO: 57
  REGION 1..306: note = engineered

SEQ ID NO: 65 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEAAPTSPIKTN

SEQ ID NO: 66 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLANGKKISINNVTEMAPTSPIKTN

SEQ ID NO: 67 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEAAPTSPIKTN

SEQ ID NO: 68 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTAEKTWYARHATLHNGKKISINNVTEMAPTSPIKTN

SEQ ID NO: 69 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYAGSVVSQVSAGVYATVRFDQYDIHNLRTIEKTWYARHATLHNGKKISINNVTEMAPTSPIKTN

SEQ ID NO: 70 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLAIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 71 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNIAQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 72 - protein, 306 aa, synthetic construct; identical to SEQ ID NO: 16
  REGION 1..306: note = engineered

SEQ ID NO: 73 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDAMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 74 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFAIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 75 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVAYTGSVVSQVSAGVYATVRFDQYDIHNLRTIEKTWYARHATLHNGKKISINNVTEMAPTSPIKTN

SEQ ID NO: 76 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGAVVSQVSAGVYATVRFDQYDIHNLRTIEKTWYARHATLHNGKKISINNVTEMAPTSPIKTN

SEQ ID NO: 77 - protein, 306 aa, synthetic construct; identical to SEQ ID NO: 22
  REGION 1..306: note = engineered

SEQ ID NO: 78 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQAATDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 79 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTAVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 80 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFAQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 81 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRAIEKTWYARHATLHNGKKISINNVTEMAPTSPIKTN

SEQ ID NO: 82 - protein, 306 aa, synthetic construct; identical to SEQ ID NO: 10
  REGION 1..306: note = engineered

SEQ ID NO: 83 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIAGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 84 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSAVSQVSAGVYATVRFDQYDIHNLRTIEKTWYARHATLHNGKKISINNVTEMAPTSPIKTN

SEQ ID NO: 85 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIAWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 86 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTAEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 87 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVAYTGSVVSQVSAGVYATVRFDQYDIHNLRTIEKTWYARHATLHNGKKISINNVTEMAPTSPIKTN

SEQ ID NO: 88 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYAGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 89 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISIANVTEMAPTSPIKTN

SEQ ID NO: 90 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFSKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 91 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGAVVSQVSAGVYATVRFDQYDIHNLRTIEKTWYARHATLHNGKKISINNVTEMAPTSPIKTN

SEQ ID NO: 92 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTAEKTWYARHATLHNGKKISINNVTEMAPTSPIKTN

SEQ ID NO: 93 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFSHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 94 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSAIKTN

SEQ ID NO: 95 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYAGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 96 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVAQVSAGVYATVRFDQYDIHNLRTIEKTWYARHATLHNGKKISINNVTEMAPTSPIKTN

SEQ ID NO: 97 - protein, 306 aa, synthetic construct; identical to SEQ ID NO: 18
  REGION 1..306: note = engineered

SEQ ID NO: 98 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSAVSQVSAGVYATVRFDQYDIHNLRTIEKTWYARHATLHNGKKISINNVTEMAPTSPIKTN

SEQ ID NO: 99 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNAVTYTGSVVSQVSAGVYATVRFDQYDIHNLRTIEKTWYARHATLHNGKKISINNVTEMAPTSPIKTN

SEQ ID NO: 100 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHASGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 101 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISIANVTEMAPTSPIKTN

SEQ ID NO: 102 - protein, 306 aa, synthetic construct; identical to SEQ ID NO: 14
  REGION 1..306: note = engineered

SEQ ID NO: 103 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTAMAPTSPIKTN

SEQ ID NO: 104 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYAAHATLHNGKKISINNVTEMAPTSPIKTN

SEQ ID NO: 105 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPAKTN

SEQ ID NO: 106 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGAVVSQVSAGVYATVRFDQYDIHNLRTIEKTWYARHATLHNGKKISINNVTEMAPTSPIKTN

SEQ ID NO: 107 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFSKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 108 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNIAQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 109 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPASPIKTN

SEQ ID NO: 110 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFAQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 111 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLAIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 112 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSANTVTYTGSVVSQVSAGVYATVRFDQYDIHNLRTIEKTWYARHATLHNGKKISINNVTEMAPTSPIKTN

SEQ ID NO: 113 - protein, 306 aa, synthetic construct; identical to SEQ ID NO: 16
  REGION 1..306: note = engineered

SEQ ID NO: 114 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIAGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 115 - protein, 306 aa, synthetic construct; identical to SEQ ID NO: 52
  REGION 1..306: note = engineered

SEQ ID NO: 116 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSAIKTN

SEQ ID NO: 117 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEAYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 118 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFSHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 119 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISIANVTEMAPTSPIKTN

SEQ ID NO: 120 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTATFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 121 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVAVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 122 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTAEKTWYARHATLHNGKKISINNVTEMAPTSPIKTN

SEQ ID NO: 123 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLANGKKISINNVTEMAPTSPIKTN

SEQ ID NO: 124 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEAGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 125 - protein, 306 aa, synthetic construct; identical to SEQ ID NO: 12
  REGION 1..306: note = engineered

SEQ ID NO: 126 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTAEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 127 - protein, 306 aa, synthetic construct; identical to SEQ ID NO: 65
  REGION 1..306: note = engineered

SEQ ID NO: 128 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPASPIKTN

SEQ ID NO: 129 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQSSTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 130 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIAWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 131 - protein, 306 aa, synthetic construct; identical to SEQ ID NO: 120
  REGION 1..306: note = engineered

SEQ ID NO: 132 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTAISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 133 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFAIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 134 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNAVTYTGSVVSQVSAGVYATVRFDQYDIHNLRTIEKTWYARHATLHNGKKISINNVTEMAPTSPIKTN

SEQ ID NO: 135 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPAKTN

SEQ ID NO: 136 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVAVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 137 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVAIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 138 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTATQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 139 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEAGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 140 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGAGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 141 - protein, 306 aa, synthetic construct; identical to SEQ ID NO: 85
  REGION 1..306: note = engineered

SEQ ID NO: 142 - protein, 306 aa, synthetic construct; identical to SEQ ID NO: 61
  REGION 1..306: note = engineered

SEQ ID NO: 143 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIAGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 144 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEAYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 145 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTAIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 146 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTAISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 147 - protein, 306 aa, synthetic construct; identical to SEQ ID NO: 105
  REGION 1..306: note = engineered

SEQ ID NO: 148 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEAYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 149 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQAASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 150 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKIAINNVTEMAPTSPIKTN

SEQ ID NO: 151 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQSSTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 152 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVAIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 153 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPAKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 154 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNASQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 155 - protein, 306 aa, synthetic construct; identical to SEQ ID NO: 67
  REGION 1..306: note = engineered

SEQ ID NO: 156 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFAIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 157 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGAGPLNSDNTVTYTGSVVSQVSAGVYATVRFDQYDIHNLRTIEKTWYARHATLHNGKKISINNVTEMAPTSPIKTN

SEQ ID NO: 158 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTAQQASTDIEWNISQPVLVPPAKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 159 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSANTVTYTGSVVSQVSAGVYATVRFDQYDIHNLRTIEKTWYARHATLHNGKKISINNVTEMAPTSPIKTN

SEQ ID NO: 160 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRAIEKTWYARHATLHNGKKISINNVTEMAPTSPIKTN

SEQ ID NO: 161 - protein, 306 aa, synthetic construct; identical to SEQ ID NO: 110
  REGION 1..306: note = engineered

SEQ ID NO: 162 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSANTVTYTGSVVSQVSAGVYATVRFDQYDIHNLRTIEKTWYARHATLHNGKKISINNVTEMAPTSPIKTN

SEQ ID NO: 163 - protein, 306 aa, synthetic construct; identical to SEQ ID NO: 130
  REGION 1..306: note = engineered

SEQ ID NO: 164 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTIEANFAHNSSTTTFQQASTDIEWNISQPVLVPPRKQVVATLVIMGGNFTIPMDAMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 165 - protein, 306 aa, synthetic construct; identical to SEQ ID NO: 128
  REGION 1..306: note = engineered

SEQ ID NO: 166 - protein, 306 aa, synthetic construct; identical to SEQ ID NO: 85
  REGION 1..306: note = engineered

SEQ ID NO: 167 - protein, 306 aa, synthetic construct; identical to SEQ ID NO: 145
  REGION 1..306: note = engineered

SEQ ID NO: 168 - protein, 306 aa, synthetic construct
  REGION 1..306: note = engineered
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTTAEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 169 - protein, 306 aa, synthetic construct
  REGION 1..306: note = engineered
  VARIANT 133: note = is not T
  VARIANT 135: note = is not E
  VARIANT 137: note = is not N
MAILDLKSLVLNAINYWGPKNNNGIQGGDFGYPISEKQIDTSIITSTHPRLIPHDLTIPQNLETIFTTTQVLTNNTDLQQSQTVSFAKKTTTATSTSTTNGWTEGGKISDTLEEKVSVSIPFIGEGGGKNSTXIXAXFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 170 - protein, 309 aa, synthetic construct
  REGION 1..309: note = engineered
  VARIANT 125: note = is not E
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGXGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 171 - protein, 309 aa, synthetic construct
  REGION 1..309: note = engineered
  VARIANT 133: note = is not T
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTXIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 172 - protein, 309 aa, synthetic construct
  REGION 1..309: note = engineered
  VARIANT 134: note = is not I
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTXEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 173 - protein, 309 aa, synthetic construct
  REGION 1..309: note = engineered
  VARIANT 135: note = is not E
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIXANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 174 - protein, 309 aa, synthetic construct
  REGION 1..309: note = engineered
  VARIANT 137: note = is not N
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEAXFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 175 - protein, 309 aa, synthetic construct
  REGION 1..309: note = engineered
  VARIANT 147: note = is not F
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTXQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 176 - protein, 309 aa, synthetic construct
  REGION 1..309: note = engineered
  VARIANT 149: note = is not Q
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQXASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 177 - protein, 309 aa, synthetic construct
  REGION 1..309: note = engineered
  VARIANT 150: note = is not A
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQXSTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 178 - protein, 309 aa, synthetic construct
  REGION 1..309: note = engineered
  VARIANT 155: note = is not E
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIXWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 179 - protein, 309 aa, synthetic construct
  REGION 1..309: note = engineered
  VARIANT 157: note = is not N
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWXISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 180 - protein, 309 aa, synthetic construct
  REGION 1..309: note = engineered
  REGION 1..309: note = is not TIC807 (SEQ ID NO: 2)
  VARIANT 12: note = X can be any naturally occurring amino acid
  VARIANT 46: note = X can be any naturally occurring amino acid
  VARIANT 52: note = X can be any naturally occurring amino acid
  VARIANT 54: note = X can be any naturally occurring amino acid
  VARIANT 68: note = X can be any naturally occurring amino acid
  VARIANT 70: note = X can be any naturally occurring amino acid
  VARIANT 87: note = X can be any naturally occurring amino acid
  VARIANT 93: note = X can be any naturally occurring amino acid
  VARIANT 95: note = X can be any naturally occurring amino acid
  VARIANT 105: note = X can be any naturally occurring amino acid
  VARIANT 117: note = X can be any naturally occurring amino acid
  VARIANT 119: note = X can be any naturally occurring amino acid
  VARIANT 125: note = X can be any naturally occurring amino acid
  VARIANT 128: note = X can be any naturally occurring amino acid
  VARIANT 133..135: note = X can be any naturally occurring amino acid
  VARIANT 137..139: note = X can be any naturally occurring amino acid
  VARIANT 145: note = X can be any naturally occurring amino acid
  VARIANT 147..151: note = X can be any naturally occurring amino acid
  VARIANT 153: note = X can be any naturally occurring amino acid
  VARIANT 155: note = X can be any naturally occurring amino acid
  VARIANT 157..159: note = X can be any naturally occurring amino acid
  VARIANT 167: note = X can be any naturally occurring amino acid
  VARIANT 175: note = X can be any naturally occurring amino acid
  VARIANT 177: note = X can be any naturally occurring amino acid
  VARIANT 180: note = X can be any naturally occurring amino acid
  VARIANT 182: note = X can be any naturally occurring amino acid
  VARIANT 187: note = X can be any naturally occurring amino acid
  VARIANT 196..201: note = X can be any naturally occurring amino acid
  VARIANT 208: note = X can be any naturally occurring amino acid
  VARIANT 217: note = X can be any naturally occurring amino acid
  VARIANT 219: note = X can be any naturally occurring amino acid
  VARIANT 223: note = X can be any naturally occurring amino acid
  VARIANT 235: note = X can be any naturally occurring amino acid
  VARIANT 239: note = X can be any naturally occurring amino acid
  VARIANT 241: note = X can be any naturally occurring amino acid
  VARIANT 243..247: note = X can be any naturally occurring amino acid
  VARIANT 249..252: note = X can be any naturally occurring amino acid
  VARIANT 273..275: note = X can be any naturally occurring amino acid
  VARIANT 282: note = X can be any naturally occurring amino acid
  VARIANT 287: note = X can be any naturally occurring amino acid
  VARIANT 293: note = X can be any naturally occurring amino acid
  VARIANT 295: note = X can be any naturally occurring amino acid
  VARIANT 299..300: note = X can be any naturally occurring amino acid
  VARIANT 303: note = X can be any naturally occurring amino acid
  VARIANT 305..306: note = X can be any naturally occurring amino acid
  VARIANT 308: note = X can be any naturally occurring amino acid
MAILDLKSLVLXAINYWGPKNNNGIQGGDFGYPISEKQIDTSIITXTHPRLXPXDLTIPQNLETIFTXTXVLTNNTDLQQSQTVSFXKKTTTXTXTSTTNGWTEXGKISDTLEEKVXVXIPFIGXGGXKNSTXXXAXXXHNSSTXTXXXXXTXIXWXXXQPVLVPPXKQVVATLXIXGGXFXIPMDXMTTIDSTEXXXXXXGYPILTXISSPDNSYXGXFMSXYFANWPNLPSGXGPLXSXNXXXXXGXXXXQVSAGVYATVRFDQYDIHNLXXXEKTWYAXHATLXNGKKIXIXNVTXXAPXSXXKXN

SEQ ID NO: 181 - DNA, 927 bp, Bacillus thuringiensis
  misc_feature 1..927: note = is a sequence representing a recombinant polynucleotide derived from a native gene from a Bacillus thuringiensis (Bt) species encoding a Cry51Aa1 protein
  source 1..927: mol_type = other DNA
atgatttttttggcaattttagatttaaaatctttagtactcaatgcaataaattattggggtcctaaaaataataatggcatacagggtggtgattttggttaccctatatcagaaaaacaaatagatacgtctattataacttctactcatcctcgtttaattccacatgatttaacaattcctcaaaatttagaaactatttttactacaactcaagtattaacaaataatacagatttacaacaaagtcaaactgtttcttttgctaaaaaaacaacgacaacaacttcaacttcaactacaaatggttggacagaaggtgggaaaatttcagatacattagaagaaaaagtaagtgtatctattccttttattggagagggaggaggaaaaaacagtacaactatagaagctaattttgcacataactctagtactactacttttcaacaggcttcaactgatatagagtggaatatttcacaaccagtattggttcccccacgtaaacaagttgtagcaacattagttattatgggaggtaattttactattcctatggatttgatgactactatagattctacagaacattatagtggttatccaatattaacatggatatcgagccccgataatagttataatggtccatttatgagttggtattttgcaaattggcccaatttaccatcggggtttggtcctttaaattcagataatacggtcacttatacaggttctgttgtaagtcaagtatcagctggtgtatatgccactgtacgatttgatcaatatgatatacacaatttaaggacaattgaaaaaacttggtatgcacgacatgcaactcttcataatggaaagaaaatatctataaataatgttactgaaatggcaccaacaagtccaataaaaacaaat

SEQ ID NO: 182 - protein, 309 aa, synthetic construct
  REGION 1..309: note = engineered
  REGION 1..309: note = is an amino acid sequence translation of nucleotide (NT) positions 1 through 927 of SEQ ID NO: 181
MIFLAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 183 - DNA, 918 bp, Bacillus thuringiensis
  misc_feature 1..918: note = is a sequence representing a recombinant polynucleotide derived from a native gene from a Bacillus thuringiensis (Bt) species encoding a TIC853 protein
  source 1..918: mol_type = other DNA
ttggcaattttagatttaaaatctttagtactcgatgcaataaactattggggtcctaaaaataataatggtatacagggttataattttaattaccctatatcagaaagacaaatagatacgtcgattataacttctactcattctcgtttaatgccacatgatttaacaattcctcaaaatttagaaactatttttactacaactcaagtattaacaaataatacagatgtacaacaaagtcaaactgtttctttttctaaaaaaacaacgacaacaacttcaacttcaactacagatggttggacagaaggtgggagaatttcagatacattagaagaaaacgtaagtgtatctattccttttattggagcgggaggagcaaaaaacagtacaactatagaagctaatgttgcacataactctagtactactacttctcaacaggcttcaactgagatagagtggaatatttcacaaccagtattggttcccccacgtaaacaagttgtagcaacattagttattatgggaggtgattttactgttcctatggatttgataactactatagattctacacaacattttactggttatccaatattaacatggatagagaaccccgagcataatgttagaggtcgatttctgagttggttttttgcaaattggcccaatttaccatcggagtttggttctttaaattcagataatacgatcacttataaaggttctgttgtaagtcgaatatcagctggtgtatatgctactgtacgatttgatcaatatgctataaataatttaagaacaattgaaaaaacttggtatgcacgacatggaactcttcataatggaaagaaaatatctataaataatgttactgaaatggcaccaacaagtccaatagaaagaaat

SEQ ID NO: 184 - protein, 306 aa, synthetic construct
  REGION 1..306: note = engineered
  REGION 1..306: note = is an amino acid sequence translation of nucleotide (NT) positions 1 through 918 of SEQ ID NO: 183
MAILDLKSLVLDAINYWGPKNNNGIQGYNFNYPISERQIDTSIITSTHSRLMPHDLTIPQNLETIFTTTQVLTNNTDVQQSQTVSFSKKTTTTTSTSTTDGWTEGGRISDTLEENVSVSIPFIGAGGAKNSTTIEANVAHNSSTTTSQQASTEIEWNISQPVLVPPRKQVVATLVIMGGDFTVPMDLITTIDSTQHFTGYPILTWIENPEHNVRGRFLSWFFANWPNLPSEFGSLNSDNTITYKGSVVSRISAGVYATVRFDQYAINNLRTIEKTWYARHGTLHNGKKISINNVTEMAPTSPIERN

SEQ ID NO: 185 - protein, 309 aa, synthetic construct
  REGION 1..309: note = engineered
  REGION 1..309: note = is TIC807 (SEQ ID NO: 2) position 11 Leucine substituted by a Methionine
MAILDLKSLVMNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 186 - DNA, 930 bp, synthetic construct
  misc_feature 1..930: note = engineered
  misc_feature 1..930: note = A nucleotide sequence encoding SEQ ID NO: 4 with an open reading frame from 1 to 927.
  source 1..930: mol_type = other DNA
atggctatcctagaccttaagtccctcgtgctgaacgccattaactactggggccctaagaacaacaacggcatccagggcggtgacttcggctaccccatctctgagaagcagatcgacactagcatcattaccttcacccaccctcgcttgatcccctacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgcgggtggcgggaagaactcgactaccatagaggccaacttcgcacacaactctagcaccactaccttccagcaagcaagcactgacattgagtggaacattagccaaccggtgctggttcctccctctaaacaagttgtcgcgacccttgtgatcatgggaggcaactttaccatccctatggacttgatgaccacgattgatagtacagagcactactcccactactccggttaccctatcctcacctggatctcgtccccagataactcttactccggtccctttatgtcatggtactttgcaaactggcctaaccttccgagtggattcggcccactgaatagtgataacacggtcacatacactggctctgtcgtgtcccaagtttcggccggtgtctacgctaccgtccggttcgatcagtatgacattcacaatctccgtactatcgagaagacttggtatgctcgccatgcgacgctgcataatggcaagaagatttctatcaacaatgtcacggaaatggctccaacatcccctatcaagacaaattga

SEQ ID NO: 187 - DNA, 930 bp, synthetic construct
  misc_feature 1..930: note = engineered
  misc_feature 1..930: note = A nucleotide sequence encoding SEQ ID NO: 6 with an open reading frame from 1 to 927.
  source 1..930: mol_type = other DNA
atggctatcctagaccttaagtccctcgtgctgaacgccattaactactggggccctaagaacaacaacggcatccagggcggtgacttcggctaccccatctctgagaagcagatcgacactagcatcattaccttcacccaccctcgcttgatcccctacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtgctggttcctccctctaaacaagttgtcgcgacccttgtgatcatgggaggcaactttaccatccctatggacttgatgaccacgattgatagtacagagcactactcccactactccggttaccctatcctcacctggatctcgtccccagataactcttactccggtccctttatgtcatggtactttgcaaactggcctaaccttccgagtggattcggcccactgaatagtgataacacggtcacatacactggctctgtcgtgtcccaagtttcggccggtgtctacgctaccgtccggttcgatcagtatgacattcacaatctccgtactatcgagaagacttggtatgctcgccatgcgacgctgcataatggcaagaagatttctatcaacaatgtcacggaaatggctccaacatcccctatcaagacaaattga

SEQ ID NO: 188 - DNA, 921 bp, synthetic construct
  misc_feature 1..921: note = engineered
  misc_feature 1..921: note = A nucleotide sequence encoding SEQ ID NO: 8 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 189 - DNA, 921 bp, synthetic construct
  misc_feature 1..921: note = engineered
  misc_feature 1..921: note = A nucleotide sequence encoding SEQ ID NO: 10 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 190 - DNA, 921 bp, synthetic construct
  misc_feature 1..921: note = engineered
  misc_feature 1..921: note = A nucleotide sequence encoding SEQ ID NO: 12 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 191 - DNA, 921 bp, synthetic construct
  misc_feature 1..921: note = engineered
  misc_feature 1..921: note = A nucleotide sequence encoding SEQ ID NO: 14 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 192 - DNA, 921 bp, synthetic construct
  misc_feature 1..921: note = engineered
  misc_feature 1..921: note = A nucleotide sequence encoding SEQ ID NO: 16 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 193 - DNA, 921 bp, synthetic construct
  misc_feature 1..921: note = engineered
  misc_feature 1..921: note = A nucleotide sequence encoding SEQ ID NO: 18 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 194 - DNA, 921 bp, synthetic construct
  misc_feature 1..921: note = engineered
  misc_feature 1..921: note = A nucleotide sequence encoding SEQ ID NO: 20 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggaggcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactag

SEQ ID NO: 195 - DNA, 921 bp, synthetic construct
  misc_feature 1..921: note = engineered
  misc_feature 1..921: note = A nucleotide sequence encoding SEQ ID NO: 22 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggcagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 196 - DNA, 921 bp, synthetic construct
  misc_feature 1..921: note = engineered
  misc_feature 1..921: note = A nucleotide sequence encoding SEQ ID NO: 24 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccgcgacctcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccttccaggaggcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacaacggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 197 - DNA, 921 bp, synthetic construct
  misc_feature 1..921: note = engineered
  misc_feature 1..921: note = A nucleotide sequence encoding SEQ ID NO: 26 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtccattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 198 - DNA, 921 bp, synthetic construct
  misc_feature 1..921: note = engineered
  misc_feature 1..921: note = A nucleotide sequence encoding SEQ ID NO: 28 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 199 - DNA, 921 bp, synthetic construct
  misc_feature 1..921: note = engineered
  misc_feature 1..921: note = A nucleotide sequence encoding SEQ ID NO: 30 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgccagtcaggtctctgccggtgtgtacgcaactgttcgcttcgatcagtatgacatccataatctcaggactattgagaagacctggtacgctcgtcatgcgacgcttcacaacggcaagaagatcagcatcaataacgtgacagaaatggcccctaccagcccgatcaagactaactga

SEQ ID NO: 200 - DNA, 921 bp, synthetic construct
  misc_feature 1..921: note = engineered
  misc_feature 1..921: note = A nucleotide sequence encoding SEQ ID NO: 32 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactaccgcccagcaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctctggactattgagaagacctggtacgctcgtcatgcgacgcttcacaacggcaagaagatcagcatcaataacgtgacagaaatggcccctaccagcccgatcaagactaactga

SEQ ID NO: 201 - DNA, 921 bp, synthetic construct
  misc_feature 1..921: note = engineered
  misc_feature 1..921: note = A nucleotide sequence encoding SEQ ID NO: 34 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactacctcccaggaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggctctgtcgtcagtcaggtctctgccggtgtgtacgcaactgttcgcttcgatcagtatgacatccataatctctggactattgagaagacctggtacgctcgtcatgcgacgcttcacaacggcaagaagatcagcatcaataacgtgacagaaatggcccctaccagcccgatcaagactaactga

SEQ ID NO: 202 - protein, 306 aa, synthetic construct; identical to SEQ ID NO: 16
  REGION 1..306: note = engineered
  REGION 1..306: note = The AA sequence translation of nucleotides 1 through 903 of SEQ ID NO: 15.

SEQ ID NO: 203 - DNA, 921 bp, synthetic construct
  misc_feature 1..921: note = engineered
  misc_feature 1..921: note = A nucleotide sequence encoding SEQ ID NO: 204 with an open reading frame from 1 to 918.
  source 1..921: mol_type = other DNA
atggctatcctagaccttaagtccctcgtgctgaacgccattaactactggggccctaagaacaacaacggcatccagggcggtgacttcggctaccccatctctgagaagcagatcgacactagcatcattacctccacccaccctcgcttgatcccccacgatcttactatcccgcagaaccttgagaccatcttcaccacaacgcaggtgctcaccaataacactgacctccagcaatcccagaccgtgagctttgcgaagaagaccactaccacgaccgcaactagcacgaccaacggttggacagaaggaggcaagatcagcgacacgctggaggagaaagtttcggttagcattccgttcatcggtgagggtggcgggaagaactcgactaccatagaggccaacttcgcacacaactctagcaccactacctcccaggaagcaagcactgacattgagtggaacattagccaaccggtccttgtgcctccccgcaaacaggttgttgccactctcgttatcatgggtggcaacttcactattcctatggatcttatgactaccattgactctactgagcactactctggctaccccattctcacttggatctcttctcctgacaatagctacagcggtcgattcatgtcatggtacttcgctaactggccgaatctcccttctggctttggtcctcttaactctgataacactgtgacctacactggccgggtcgaaagtcgggtctctgccggtgtgtacgcaactgttcgcttcgatcagtatgacatccataatctctggactattgagaagacctggtacgctcgtcatgcgacgcttcacaacggcaagaagatcagcatcaataacgtgacagaaatggcccctaccagcccgatcaagactaactga

SEQ ID NO: 204 - protein, 306 aa, synthetic construct
  REGION 1..306: note = engineered
  REGION 1..306: note = The amino acid sequence translation of the open reading frame set forth as SEQ ID NO: 203.
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTSQEASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGRVESRVSAGVYATVRFDQYDIHNLWTIEKTWYARHATLHNGKKISINNVTEMAPTSPIKTN

SEQ ID NO: 205 - DNA, 2577 bp, Bacillus thuringiensis
  misc_feature 1..2577: note = AXMI-171 US2010/0298207 A1 SEQ ID NO:204
  source 1..2577: mol_type = unassigned DNA
atgaaaaataaaaagaaatatatgaagccactcgcagtaggcttgctcgcaactaacattattggatttggtactcagacagtagcgtttgctgcgacagataaagcgggttccaaagaacagatgcagcaacaaatgaaaacacagaataagagctttaatccgacagtattagccagtatgccatcggatacatcggagcttcaaaaaatgttggaagacgcgattaaaaataaggatacccagattactccggaattaataaaaaaacttcaggacaaaggatttgattaccttagtattgtaaagggtttaactggcggattactgaagcaaattccgtatgcaggttcaatcctctcccctttggtagttggcctttttccaggtaagggttatgtaacgaaggcgaatgtctggggagagatacaggatcgcgtttcaaatttaatagatcaaaaattagaagagtcacaagtaaataacttaattgggaaactaacgggtattcaggataatttaggaatataccaaactcgggttggtttagttaatgggataaaaccaccaattgctaattttatacaaaaggatgcaaattctgataaaaataaggagaatttaagaagcacaatagactccttggacaaggatttaggccgagtgatacctgagtttgctgttaaaggttacgaggcagcttctcttccatattatgtacaagttgccaatgtgcatcttttcttattgaaagatgcacttacacatgcagatgagtgggggcttactgatgatgaaaagagaggatatttgtcaagacttcaacaaaaaattcaagagtatagcagcgtcgtatatgattcctttaataaaggagtcgaagccgctaaaagtaagggtggaagtaccgccgacagttggaacagaaccaatgcctatgtaagaacaatgacattgtatggtttagattttgtggctttatggccggcttttgatactaaacattataatcagccagtaaaattgcagcaaacacgtgaattatattctaacatgataggaagaccaataaactggcaagactatgatacaactcttcaacaaattcataatagtgggtatgcgggttatccaggcgagctgaaacaagttggtgtttcacagtgggatcgtattgatggaatcagggaaatatttgattggactggagacggctcacgagattatacgctacaatggggccacgcaaacaaaaatgggtattcggaccgtagccagaccgtcaataacccagcaataggaatttcagcatatgaatccaataatgctaatttctataatatgtctactatcacatataaacaaaataacgaagtatcttggttctatggtccatttactactcaaagcgatagtaaagatggaagtagaatagatagcaaagctccagcgggccataaattatctcgcgtcaaagtacaagaaaaaagatcagatctaaatacaatatcatcttttgtggctgcatatgttcctgaagaagttcatccacagaatatacttgaggctaaagctattacaggggtaccagctgaaaaatatctcgcacatgcaggatttgaagataagattgaatacatgaatggttcgaatgcgatggtatcttctaaaaatggtgacacgatagattacaatgttcaaagtccaggaaaacaaaaatataaaatacgcctccgtgttgcgacaaatagtgacacatctgtgggaatctctataaatggggattctcagcaagtgaatataaaaaatacagaagccgcaacaaagttagaagacggtattacagttaaaggtgtaaatgggaagtacatgttaattgacggaccaactgttgaacttagcgaaggtgtgaatacaattcagcttaaaaatagtggtggagctaagattgcattagatcgaatagaatttgagcctataggcggtgagctacgcaagtggaaacaggaaggtgataagtggtacttttacgatgaaaatgataaaaagttaacaggttggcaaaagattaatgaaagaaaatactaccttggacattctggtgatggttctggtatgaccactgaaggggagatggcaacaggctggaaaacgattgatggagtacaatattactttggacattctggtgatggttctggtatggtcactgaaggggagatggcaacaggctggaaaacgataaatggagtaaaatattattttggacagactggtgatggctctggtatgcaacacgaaggggaaaaggcaacaggctggaaaacgattgatggagtaaaatattacttcaataaaactggtgatggctctggtatgcaacacgaaggggaaaaggcaataggctggaaaacgattgatggagtaaaatattacttcaataaaactggtgatggctctggtatgcaacacgaaggagaaatggcattaggagatatgacgattgatggagtaaaacatcactttaataaaactggtgatggaacgggtcgcgaccatgaaggagagctcgtatgg

SEQ ID NO: 206 - protein, 859 aa, Bacillus thuringiensis
  REGION 1..859: note = AXMI-171 US2010/0298207 A1 SEQ ID NO:205
MKNKKKYMKPLAVGLLATNIIGFGTQTVAFAATDKAGSKEQMQQQMKTQNKSFNPTVLASMPSDTSELQKMLEDAIKNKDTQITPELIKKLQDKGFDYLSIVKGLTGGLLKQIPYAGSILSPLVVGLFPGKGYVTKANVWGEIQDRVSNLIDQKLEESQVNNLIGKLTGIQDNLGIYQTRVGLVNGIKPPIANFIQKDANSDKNKENLRSTIDSLDKDLGRVIPEFAVKGYEAASLPYYVQVANVHLFLLKDALTHADEWGLTDDEKRGYLSRLQQKIQEYSSVVYDSFNKGVEAAKSKGGSTADSWNRTNAYVRTMTLYGLDFVALWPAFDTKHYNQPVKLQQTRELYSNMIGRPINWQDYDTTLQQIHNSGYAGYPGELKQVGVSQWDRIDGIREIFDWTGDGSRDYTLQWGHANKNGYSDRSQTVNNPAIGISAYESNNANFYNMSTITYKQNNEVSWFYGPFTTQSDSKDGSRIDSKAPAGHKLSRVKVQEKRSDLNTISSFVAAYVPEEVHPQNILEAKAITGVPAEKYLAHAGFEDKIEYMNGSNAMVSSKNGDTIDYNVQSPGKQKYKIRLRVATNSDTSVGISINGDSQQVNIKNTEAATKLEDGITVKGVNGKYMLIDGPTVELSEGVNTIQLKNSGGAKIALDRIEFEPIGGELRKWKQEGDKWYFYDENDKKLTGWQKINERKYYLGHSGDGSGMTTEGEMATGWKTIDGVQYYFGHSGDGSGMVTEGEMATGWKTINGVKYYFGQTGDGSGMQHEGEKATGWKTIDGVKYYFNKTGDGSGMQHEGEKAIGWKTIDGVKYYFNKTGDGSGMQHEGEMALGDMTIDGVKHHFNKTGDGTGRDHEGELVW